(19) United States
(12) Patent Application Publication
(10) Pub. No.: US 2021/0263046 A1
(43) Pub. Date: Aug. 26, 2021

(54) Antibodies and Immunoassays for Detection of BMP-15 and GDF-9 Isoforms

(71) Applicant: Ansh Labs LLC, Webster, TX (US)

(72) Inventors: Gopal V. SAVJANI, El Lago, TX (US); Ajay KUMAR, Friendswood, TX (US); Bhanu KALRA, Friendswood, TX (US)

(21) Appl. No.: 16/497,046

(22) PCT Filed: Mar. 26, 2018
(86) PCT No.: PCT/US18/24321

§ 371 Date: Sep. 24, 2019

Related U.S. Application Data
(60) Provisional application No. 62/476,227, filed on Mar. 24, 2017.

(51) Int. Cl. G01N 33/68

(52) U.S. Cl. CPC G01N 33/6893; G01N 2800/367; G01N 2333/51; G01N 2333/495

(57) ABSTRACT

Methods for quantifying GDF-9-BMP-15 heterodimers and GDF-9 and BMP-15 homodimers using antibodies directed to epitopes in GDF-9 and BMP-15 in a sandwich ELISA are provided. Also provided are kits for quantifying the heterodimers.

### BACKGROUND

TGF-β (transforming growth factor-beta) superfamily is the largest family of secreted proteins in mammals and members of the TGF-β family are involved in most developmental and physiological processes. Bone morphogenetic protein 15 (BMP-15), also known as GDF-9B, is a protein of the TGF-β superfamily. In humans, it is encoded by the BMP15 gene. Proteins of this superfamily bind various TGF-β receptors leading to recruitment and activation of SMAD family transcription factors and regulation of gene expression. BMP-15 is encoded as a preproprotein which is proteolytically processed to generate two fragments, a pro fragment and a mature fragment, each of which can self-associate to form a disulfide-linked homodimer. Growth differentiation factor 9 (GDF-9), an analog of BMP-15, is also encoded as a preproprotein, and like BMP-15 is proteolytically processed to generate two fragments which, similar to BMP-15, form disulfide-linked homodimers through self-association. The amino acid sequence of mature human BMP-15 is 70%, 68%, and 78% identical, respectively, to amino acid sequences of mouse, rat, and sheep mature BMP-15. Further, mature BMP-15 shares 27% to 38% amino acid sequence identity with other BMPs.

BMP-15 and GDF-9 are distinguishable from other markers of ovarian function such as AMH, inhibin A, inhibin B, and E2, in that they are produced specifically by oocytes, whereas the latter group of molecules are produced by granulosa cells. As such, BMP-15 and GDF-9 may provide a more direct assessment of oocyte function.

BMP-15 plays a major role in many aspects of ovarian development. In a recent review, Persani¹ listed the biological actions of BMP-15 as (i) promotion of follicle growth and maturation starting from the primary gonadotrophin-independent phases of folliculogenesis, (ii) regulation of follicular granulosa cell (GC) sensitivity to FSH action and determination of ovulation quota, (iii) prevention of GC apoptosis, and (iv) promotion of oocyte developmental competence. The latter is key in reproductive medicine as it refers to the capacity of the oocyte to support early embryo development and is a reflection of intrinsic oocyte quality.

Current methods of determining oocyte quality are not very reliable. As regards determining oocyte quality based on levels of BMP-15 and/or GDF-9, account should be taken of the fact that both undergo intracellular processing to generate a pro and a mature fragments, which can combine in multiple ways to produce several isoforms. Thus, it is not clear which isoform is being measured by a given assay. For example, BMP-15 is synthesized with a 249 amino acid (aa) N-terminal propeptide². Upon cleavage, a 50 kDa pro BMP-15 polypeptide is produced. However, it appears that the mature dimer is stabilized by remaining in association with the pro region³. Mature BMP-15 exists in 16 kDa and 17 kDa forms which are distinguishable by the presence of O-linked glycosylation on the 17 kDa form³. Further, mature BMP-15 is phosphorylated, a modification which is required for the stimulation of GC proliferation⁴. Mature GDF-9 and mature BMP-15 form 40 kDa and 34 kDa noncovalently-linked homodimers, respectively, and 37 kDa heterodimers. Both are bioactive. BMP-15 exerts its effects through interactions with BMPR-IB/ALK6 and BMPR-II receptors³⁻⁵. Further, GDF-9 and BMP-15 synergize in promoting oocyte survival and folliculogenesis⁶⁻⁷.

Thus, in order to determine oocyte quality using, e.g., BMP-15 and GDF-9, there is a need to develop improved methods for determining levels of these two protein or those of the complexes formed by them.

### SUMMARY OF THE INVENTION

The present invention provides immunoassays for determining levels of GDF-9, BMP-15 and GDF-9-BMP-15 heterodimeric complex in a sample from a mammalian subject. These immunoassays are helpful in diagnosing or prognosing a disease or condition such as granulosa cell tumors, disorders of sex development and also for diagnosing ovarian reserve, ovarian insufficiency, predicting time to menopause, and selection of oocytes for in vitro development. Exemplary embodiments of the invention are summarized as numbered items in the following.

Item 1. A method of quantifying GDF-9-BMP-15 heterodimers in a sample, the method comprising:

performing an immunoassay on the sample using a first antibody that specifically binds to an epitope of GDF-9 and a second antibody that specifically binds to an epitope of BMP-15, wherein one of said first and second antibodies is used for the capture and the other is used for the detection step of the immunoassay;

measuring a detection signal generated by an agent conjugated to the detection antibody; and

calculating the amount of GDF-9-BMP-15 heterodimers in the sample by comparing the detection signal to a calibration curve correlating an amount of GDF-9-BMP-15 heterodimer to the detection signal.

Item 2. The method of item 1, wherein the immunoassay is a sandwich ELISA.

Item 3. The method of item 1 or item 2, wherein the detection comprises measuring a fluorescence signal.

Item 4. The method of item 1 or item 2, wherein the detection comprises measuring a chemiluminiscence signal.

Item 5. The method of any one of items 1-4, wherein the sample is derived from a mammalian subject.

Item 6. The method of any of items 1-5, wherein the mammalian subject is a human.

Item 7. The method of any of items 1-6, wherein the sample is follicular fluid.

Item 8. The method of any of items 1-7, wherein the immunoassay is performed in the presence of one or more protein dissociating agents.

Item 9. The method of item 8, wherein the one or more protein dissociating agents are selected from the group consisting of Triton-X 100, guanidinium chloride, sodium dodecyl sulfate (SDS), urea, thiourea, lithium perchlorate, lithium acetate, and magnesium chloride.

Item 10. The method of item 9, wherein the immunoassay is performed in the presence of 0.1%-0.3% SDS and 0.5%-1.5% Triton-X 100.

Item 11. The method of item 10, wherein the immunoassay is performed in the presence of 0.25% SDS and 1.0% Triton-X 100.

Item 12. The method of item 10, wherein the immunoassay is performed in the presence of 0.125% SDS and 1.0% Triton-X 100.

Item 13. The method of any one of items 1-12, wherein the first antibody specifically binds to an epitope of GDF-9 contained in an amino acid sequence selected from SEQ ID NOS: 111, 119, 120, 123-125, 128-136, and 144-146; and wherein the second antibody binds to an epitope of BMP-15 contained in an amino acid sequence selected from SEQ ID NOS: 8-12, 28, 30, 32-34, 40, 41, 45, 46, 48-51, 55, 61, and 68.

Item 14. The method of item 13, wherein the first antibody specifically binds to an epitope of GDF-9 contained in an amino acid sequence selected from SEQ ID NOS: 111, 119, 120, 123-125, 128-136, and 144-146; and the second antibody binds to an epitope of BMP-15 contained in an amino acid sequence selected from SEQ ID NOS: 8, 9, 12, 32, and 33.

Item 15. A method of quantifying BMP-15 homodimer in a sample, the method comprising performing an immunoassay on the sample using the same anti-BMP-15 antibody for both the capture and the detection steps of the immunoassay, wherein the antibody specifically binds an epitope contained in an amino acid sequence selected from SEQ ID NOS: 8-12, 28, 30, 32-34, 40, 41, 45, 46, 48-51, 55, 61, and 68;

measuring a detection signal generated by an agent conjugated to the anti-BMP-15 antibody used in the detection step; and

calculating the amount of BMP-15 homodimer in the sample by comparing the detection signal to a calibration curve correlating an amount of BMP-15 homodimer to the detection signal.

Item 15. The method of item 15, wherein the immunoassay is a sandwich ELISA.

Item 17. The method of item 15 or item 16, wherein the detection comprises measuring a fluorescence signal.

Item 18. The method of item 15 or item 16, wherein the detection comprises measuring a chemiluminiscence signal.

Item 19. The method of any of items 15-18, wherein the antibody specifically binds an epitope contained in an amino acid sequence selected from SEQ ID NOS: 8 and 9.

Item 20. The method of any of items 15-18, wherein the antibody specifically binds an epitope contained in an amino acid sequence selected from SEQ ID NOS: 32 and 33.

Item 21. A method of quantifying GDF-9 homodimer in a sample, the method comprising:

performing an immunoassay on the sample using the same anti-GDF-9 antibody for both the capture and the detection steps of the immunoassay, wherein the antibody specifically binds an epitope contained in an amino acid sequence selected from SEQ ID NOS: 111, 119, 120, 123-125, 128-136, and 144-146;

measuring a detection signal generated by an agent conjugated to the anti-GDF-9 antibody used in the detection step; and

calculating the amount of GDF-9 homodimer in the sample by comparing the detection signal to a calibration curve correlating an amount of GDF-9 homodimer to the detection signal.

Item 22. The method of item 21, wherein the immunoassay is a sandwich ELISA.

Item 23. The method of item 21 or item 22, wherein the detection comprises measuring a fluorescence signal.

Item 24. The method of item 21 or item 22, wherein the detection comprises measuring a chemiluminiscence signal.

Item 25. The method of any of items 15-24, wherein the immunoassay is performed in the presence of one or more protein dissociating agents.

Item 26. The method of item 25, wherein the one or more protein dissociating agents are selected from the group consisting of Triton-X 100, guanidinium chloride, sodium dodecyl sulfate (SDS), urea, thiourea, lithium perchlorate, lithium acetate, and magnesium chloride.

Item 27. The method of item 26, wherein the immunoassay is performed in the presence of 0.1%-0.3% SDS and 0.5%-1.5% Triton-X 100.

Item 28. The method of item 26, wherein the immunoassay is performed in the presence of 0.25% SDS and 1.0% Triton-X 100.

Item 29. The method of item 26, wherein the immunoassay is performed in the presence of 0.125% SDS and 1.0% Triton-X 100.

Item 30. A kit for quantifying GDF-9-BMP-15 heterodimers, the kit comprising an anti-GDF-9 antibody, an anti BMP-15 antibody, wherein the anti-GDF-9 antibody specifically binds an epitope contained in an amino acid sequence selected from SEQ ID NOS: 111, 119, 120, 123-125, 128-136, and 144-146, and wherein the anti-BMP-15 antibody specifically binds to an epitope contained in an amino acid sequence selected from SEQ ID NOS: 8-12, 28, 30, 32-34, 40, 41, 45, 46, 48-51, 55, 61, and 68.

Item 31. A method of quantifying BMP-15 homodimer in a sample, the method comprising performing an immunoassay on the sample using the same anti-BMP-15 antibody for both the capture and the detection steps of the immunoassay;

measuring a detection signal generated by an agent conjugated to the anti-BMP-15 antibody used in the detection step; and

calculating the amount of anti-BMP-15 homodimer in the sample by comparing the detection signal to a calibration curve correlating an amount of BMP-15 homodimer to the detection signal.

Item 32. The method of item 31, wherein the immunoassay is a sandwich ELISA.

Item 33.The method of item 31 or item 32, wherein the anti-BMP-15 antibody recognizes an epitope contained in the mature region of BMP-15.

Item 34. The method of any of items 31-33, wherein a mixture of pro-mature and mature-mature form of BMP-15 homodimer is quantified.

Item 35. The method of item 33 or item 34, wherein the anti-BMP-15 antibody is secreted by clone 128/98A and identified by identifier 23A.

Item 36. The method of item 31 or item 32, wherein the anti-BMP-15 antibody recognizes an epitope contained in the pro region of BMP-15.

Item 37. A method of quantifying a mixture of mature-mature and pro-mature GDF-9 in a sample, the method comprising:

performing an immunoassay on the sample using a first and a second anti-GDF-9 antibody, each antibody binding specifically to an epitope of GDF-9, wherein one of said first and second antibodies is used for the capture and the other is used for the detection step of the immunoassay;

measuring a detection signal generated by an agent conjugated to the detection antibody; and

calculating the amount of the mixture of mature-mature and pro-mature GDF-9 in the sample by comparing the detection signal to a calibration curve correlating an amount of pro-mature GDF-9 to the detection signal.

Item 38. The method of item 37, wherein the immunoassay is a sandwich ELISA.

Item 39. The method of item 37 or item 38, wherein

the first antibody is (i) secreted by clone 114/58 and identified by identifier 17A, and (ii) used for capture; and

the second antibody is (i) secreted by clone 98/74 and identified by identifier 6, (ii) used for detection, and (iii) specifically binds an epitope contained in an amino acid sequence selected from SEQ ID NOS: 123, 129, 132, and 144-146.

Item 40. A method of quantifying pro-mature GDF-9 in a sample, the method comprising:

performing an immunoassay on the sample using a first and a second anti-GDF-9 antibody, each antibody binding specifically to an epitope of GDF-9, wherein one of said first and second antibodies is used for the capture and the other is used for the detection step of the immunoassay;

measuring a detection signal generated by an agent conjugated to the detection antibody; and

calculating the amount of pro-mature GDF-9 in the sample by comparing the detection signal to a calibration curve correlating an amount of pro-mature GDF-9 to the detection signal.

Item 41. The method of item 40, wherein the immunoassay is a sandwich ELISA.

Item 42. The method of item 40 or 41, wherein

the first antibody is (i) secreted by clone 99/69 and identified by identifier 25, and (ii) used for capture; and

the second antibody is (i) secreted by clone 98/74 and identified by identifier 6, (ii) used for detection, and (iii) specifically binds an epitope contained in an amino acid sequence selected from SEQ ID NOS: 123, 129, 132, 144-146.

Item 43. A method of quantifying mature-mature GDF-9 in a sample, the method comprising subtracting the amount of pro-mature GDF-9 in the sample as determined by the method of item 42 from the amount of the mixture of mature-mature and pro-mature GDF-9 in the sample as determined by the method of item 39.

Item 44. A method to aid in diagnosing or prognosing a disease or condition selected from the group consisting of: granulosa cell tumors, disorders of sex development, polycystic ovarian syndrome, gonadotoxicity; the method comprising quantifying in a biological sample from the subject one or more of (i) GDF-9-BMP-15 heterodimers according to any of items 1-14, (ii) BMP-15 homodimer according to any of items 15-20, and (iii) GDF-9 homodimers according to any of items 21-29 or item 43.

Item 45. The method of item 44, wherein the disorder of sex development is selected from conditions of newborns with atypical genitalia, conditions of adolescents presenting atypical sexual development, cryptorchidism, and abnormal testicular function.

Item 46. The method of item 44, wherein the gonadotoxicity is induced by chemotherapy.

Item 47. A method of determining ovarian reserve in a female human subject, the method comprising quantifying in a biological sample from the subject one or more of (i) GDF-9-BMP-15 heterodimers according to any of items 1-14, (ii) BMP-15 homodimer according to any of items 15-20, and (iii) GDF-9 homodimers according to any of items 21-29 or item 43.

Item 48. The method according to item 47, further comprising comparing the quantified GDF-9-BMP-15 heterodimers, or BMP-15 homodimer, or GDF-9 homodimers to a standard that correlates GDF-9-BMP-15 heterodimers. or BMP-15 homodimer, or GDF-9 homodimers, respectively, to a number of oocytes as a measure of ovarian reserve.

Item 49. The method of item 47 wherein the sample is obtained prior to ovulation induction in the subject.

Item 50. A method to aid in diagnosing ovarian insufficiency, the method comprising quantifying in a biological sample from the subject one or more of (i) GDF-9-BMP-15 heterodimers according to any of items 1-14, (ii) BMP-15 homodimer according to any of items 15-20, and (iii) GDF-9 homodimers according to any of items 21-29 or item 43.

Item 51. A method of predicting time to menopause in a female human subject, the method comprising quantifying in a biological sample from the subject one or more of (i) GDF-9-BMP-15 heterodimers according to any of items 1-14, (ii) BMP-15 homodimer according to any of items 15-20, and (iii) GDF-9 homodimers according to any of items 21-29 or item 43.

Item 52. A method for selecting oocytes for in vitro maturation, the method comprising quantifying in a biological sample from the subject one or more of (i) GDF-9-BMP-15 heterodimers according to any of items 1-14, (ii) BMP-15 homodimer according to any of items 15-20, and (iii) GDF-9 homodimers according to any of items 21-29 or item 43.

### BRIEF DESCRIPTION OF THE DRAWINGS

FIG. 1 is a schematic diagram showing proteolytic processing of BMP-15 and GDF-9. The processing results in a pro and a mature form of each protein. The pro and mature forms of each can associate with themselves or with another form giving rise to one of several homodimeric or heterodimeric complexes.

FIG. 2 is an immunohistochemistry (IHC) image of a human formalin fixed human ovary section stained with the anti-GDF-9 antibody 7 (clone 98/68 A) (5 μg/mL, Tris-buffer, DAB 10 min., haematoxylin 5 min).

FIGS. 3A and 3B are IHC images of formalin fixed human ovary sections stained with anti-GDF-9 antibody 10A (clone 114/62 A) (10 μg/mL, citrate buffer, DAB 2½ min., Mayer 2 min). FIG. 3B is a higher magnification of the image of FIG. 3A.

FIG. 4 is a set of IHC images of formalin fixed mouse ovary tissue sections stained with anti-GDF-9 antibodies produced by clones 98/68, 98/74, 114/62, 114/5, and 114/31 (5 ug/mL). The antibodies are designated 7, 6, 10A, 12A, and 14A, respectively.

FIGS. 5A and 5B are IHC images of formalin fixed mouse ovary tissue sections stained with anti-GDF-9 antibodies produced by clones 111/49 A and111/53A, respectively (5 ug/mL, citrate buffer, DAB 5 min., Mayer 2 min).

FIG. 6 is a set of IHC images of formalin fixed mouse ovary tissue sections stained with BMP-15 antibodies 20 (clone 111/1A) and 19 (clone 111/77A).

FIGS. 7A and 7B show results of western blot analysis performed using a set of anti-GDF-9 antibodies. FIG. 7A shows 54 kDa pro-mature and 16 kDa monomer complexes recognized by one or more of the antibodies. FIG. 7B is a positive control and shows recognition of recombinant BMP-15 monomer by the antibody (12A) secreted by clone 114/5.

FIG. 8 shows results of western blot analysis performed using a set of anti-BMP-15 antibodies.

FIG. 9 is a graph showing the relationship between increasing concentrations of full-length recombinant BMP-15 (pro+mature) and signals detected using an ELISA based on using the anti-BMP-15 antibody 23A (clone 128/98A) for both capture and detection purposes. This antibody is specific for an epitope in the mature region of BMP-15.

FIG. 10 is a graph showing the relationship between increasing concentrations of full-length recombinant BMP-15 (pro+mature) and signals detected using an ELISA based on using the anti-BMP-15 antibodies 21 (clone 111-39A) and 4 (clone 87-38A), both specific to epitopes in the pro region of BMP-15.

FIG. 11 is a graph showing the relationship between increasing concentrations of full-length recombinant GDF-9 (pro+mature) and signals detected using an ELISA based on using the anti-GDF-9 antibodies 17A (clone 114-58A) and 6 (clone 98-74A), both specific to epitopes in the pro region of BMP-15.

FIG. 12 is a graph showing the relationship between increasing concentrations of recombinant GDF-9 and detection signals using an ELISA described in Example 9.

FIG. 13 is a graph showing results of the effect on the measurement of GDF-9 (pr+mature) upon spiking a sample of GDF-9 (pro+mature) with the proteins shown on the right.

FIG. 14 is another graph showing results of the effect on the measurement of GDF-9 (pro+mature) upon spiking a sample of GDF-9 (pro+mature) with the proteins shown on the right.

FIG. 15 is a graph showing the relationship between increasing concentrations of BMP-15-GDF-9 complex and detection signals using an ELISA described in Example 11.

FIGS. 16A-16S are graphs showing binding of various anti-GDF-9 antibodies to peptides corresponding to epitopes of GDF-9.

FIGS. 17A-17Z and 17A1-17A2 are graphs showing binding of various anti-BMP-15 antibodies to peptides corresponding to epitopes of BMP-15.

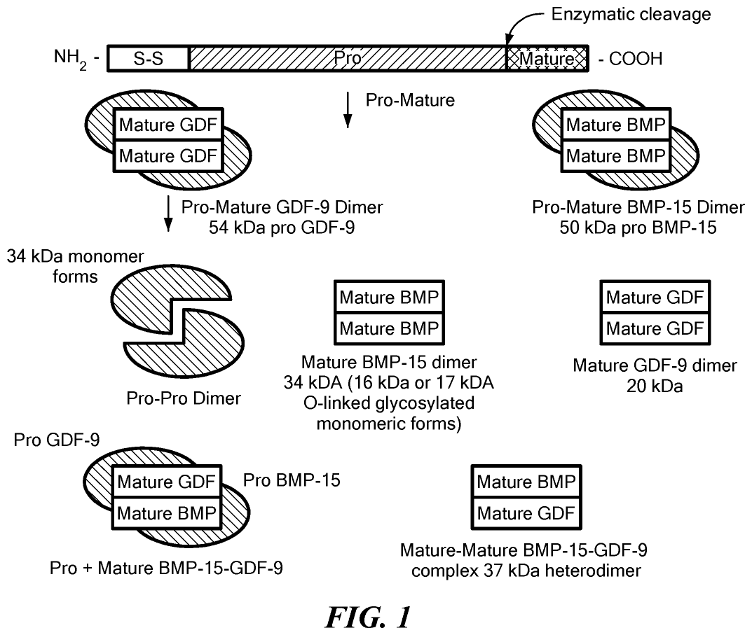

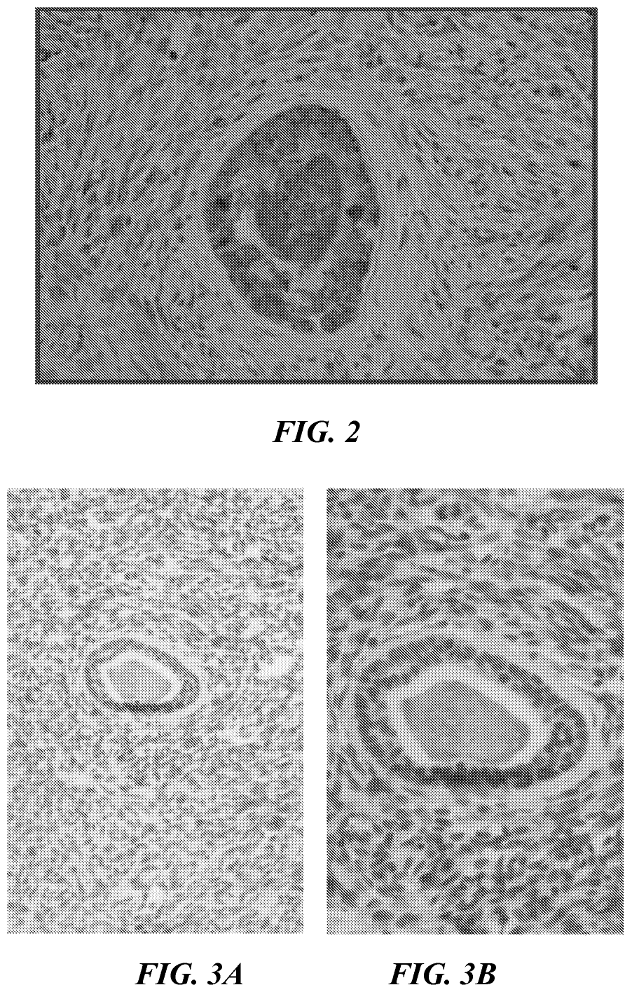

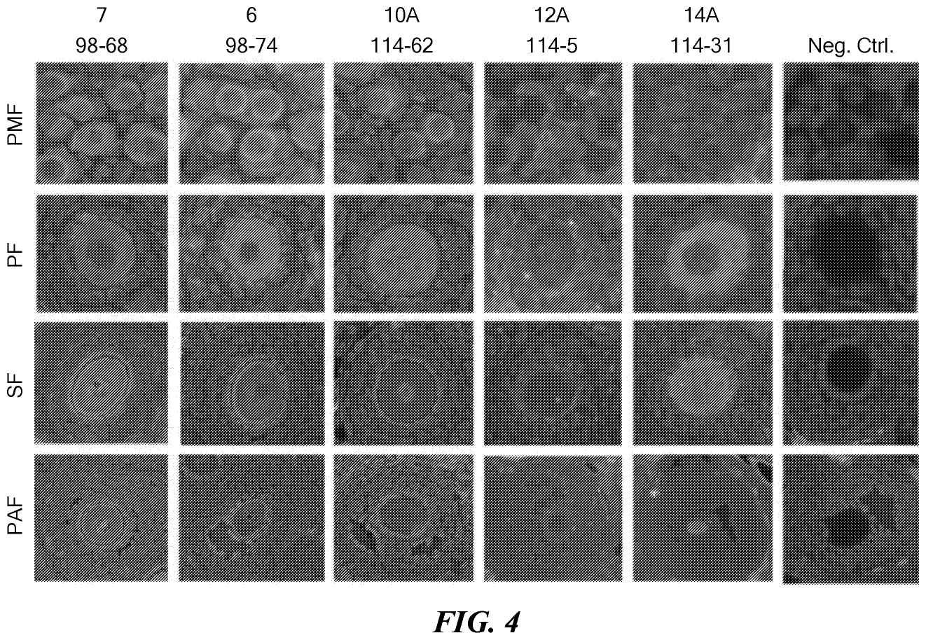

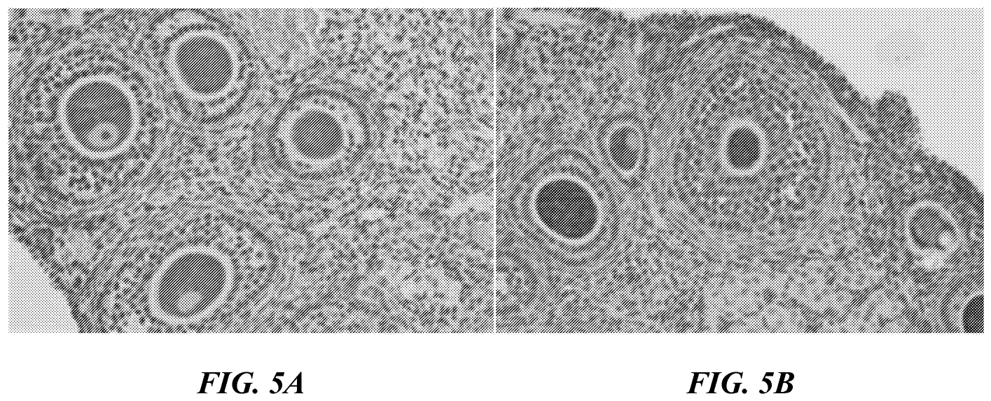

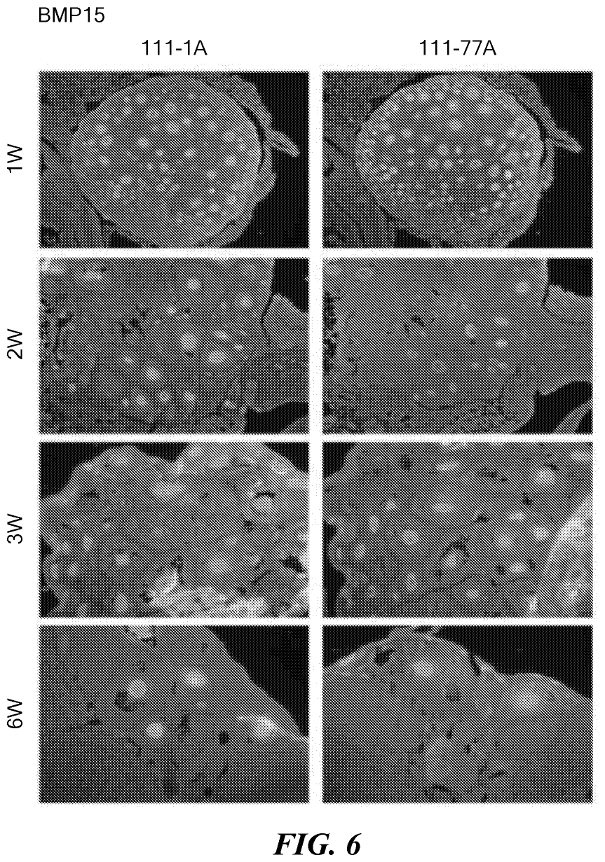

### DETAILED DESCRIPTION OF THE INVENTION

The invention provides methods for quantifying TGF-beta family members GDF-9 and BMP-15 and heterodimeric complexes formed by interactions between the two. Unlike other known markers of ovarian function (e.g., AMR, inhibin A, inhibin B, and E2), which are derived from granulosa cells (GC), GDF9 and BMP-15 are derived specifically from oocytes. Both GDF-9 and BMP-15 are produced as pro-peptides and undergo proteolytic cleavage inside the cell to generate pro and mature forms (fragments). The mature fragments of GDF-9 and BMP-15 can form non-covalent bioactive homo and hetero dimers. BMP-15 and GDF-9 pro-peptides are synthesized as precursors having 249-295 N-terminal pro domains and 125-135 C-terminal mature domains. GDF-9 and BMP-15 form 40 kDa and 34 kDa homodimers and 37 kDa heterodimers. Recent evidence shows that GDF-9-BMP-15 is a highly active GDF-9-like superagonist (1000-fold more potent than GDF-9 itself).

In accordance with the invention, a method for quantifying GDF-9-BMP-15 heterodimer is provided. The method includes performing a sandwich ELISA on a sample, e.g., a follicular fluid sample, using a first antibody for capture and a second antibody for detection. The first antibody and the second antibody bind specifically to an epitope of GDF-9 and BMP-15, respectively. A signal is generated by an agent conjugated to the second antibody. This signal is measured and the amount of GDF-9-BMP-15 heterodimer is calculated by comparing it to a calibration curve correlating an amount of GDF-9-BMP-15 heterodimer to the detection signal.

As used herein “GDF-9-BMP-15 heterodimer” refers to a heterodimeric complex formed between GDF-9 (uncleaved pro+mature or cleaved-reassociated pro+mature) and BMP-15 (uncleaved pro+mature or cleaved but reassociated pro+mature).

A unique feature of the method is that it can be performed in the presence of agents that under certain concentrations, cause partial unfolding of the GDF-9-BMP-15 heterodimeric complex or of the GDF-9 or BMP-15 pro-mature homodimeric complex such that certain epitopes in the mature region are sufficiently exposed and can be accessed by particular mature region specific antibodies, enabling detection of the complex using these antibodies. Importantly, these antibodies are not affected by the presence of the agent(s). Given that (i) the mature region is much smaller compared to the pro region, and that (ii) even after cleavage the pro and mature forms remain associated, to a great extent access of antibodies to the mature region is blocked by the pro region. As such, the ability to partially unfold the pro-mature complex formed by BMP-15 and GDF-9, such that one or more epitopes in the mature region are exposed and can be detected using an antibody is of great significance for the development of methods for determining ovarian function/quality.

Methods are also provided for quantifying either GDF-9 (or BMP-15) homodimers in a sample. These methods require performing a sandwich ELISA on the sample using an anti-GDF-9 (or an anti-BMP-15 antibody) for both capture and detection, wherein the antibody specifically binds an epitope of GDF-9 (or BMP-15). Using this method, GDF-9 and BMP-15 were quantitated by sandwich ELISAs based on use of pairs of monoclonal antibodies (mAb) for capturing and detecting mature or pro portions of GDF-9 and BMP15. Mammalian cell-derived recombinant proteins were used as calibrators.

For example, an ELISA was performed with a pair of antibodies recognizing epitopes in the mature region of GDF-9 (mature/mature GDF-9). The analytical measuring range (AMR) and the limit of detection (LoD) of this ELISA was found to be 35-4000 pg/mL and 5 pg/mL, respectively. This assay detects human pro-mature GDF-9 complex, mature GDF-9 complex, and GDF-9-BMP-15 complex. Spiking of follicular fluid with mature GDF-9 or mature GDF-9-BMP-15 showed results similar to that obtained with spiking with pro-mature GDF-9.

Additionally, assays were performed with pairs of antibodies recognizing epitopes in the pro region of GDF-9 (pro/pro GDF-9; AL-177) or in the pro and mature regions of GDF-9 (pro/mature GDF-9; AL-178). These assays were found to be specific for detecting human pro-mature GDF-9 and did not detect mature GDF-9 dimers.

Further, an assay based on a pair of antibodies recognizing epitopes in the mature region of BMP-15 (mature/mature BMP-15; AL-179) was performed. This assay was found to measure pro-mature and mature BMP-15 but not GDF-9-BMP-15 complex. The AMR and LoD of this assay is 2.7-336 pg/mL and 0.4 pg/mL, respectively.

Further still, an assay utilizing pairs of antibodies recognizing the pro and the-mature regions of BMP-15 (pro/mature BMP-15; AL-180) was performed. This assay detected pro-mature BMP-15 but not mature BMP-15 dimer or the GDF-9-BMP-15 complex.

Two more assays that recognize heterodimers of GDF-9 and BMP-15 were performed. These assays were found to and have an AMR of 0.35-40 ng/mL and LoD of 53 pg/mL.

Using the above assays, GDF9, BMP-15 and GDF-9-BMP-15 concentrations in human mature follicles (n=28) were found to be in the range of 5-632 pg/mL, 0.4-2.1 pg/mL, 0.5-135 pg/mL, respectively.

The antibodies and methods described in the present invention may be used in diagnosing or prognosing a disease or condition such as granulosa cell tumors, disorders of sex development, polycystic ovarian syndrome, gonadotoxicity. Disorders of sex development may include conditions of newborns with atypical genitalia, conditions of adolescents presenting atypical sexual development, cryptorchidism, and abnormal testicular function. Further, these antibodies and methods may be used for diagnosing ovarian reserve, ovarian insufficiency, predicting time to menopause, and selection of oocytes for in vitro development. Without further elaboration, it is believed that one skilled in the art can, based on the description above, utilize the present invention to its fullest extent. The specific examples below are to be construed as merely illustrative and not limitative of the remainder of the disclosure in 15 any way whatsoever.

### EXAMPLES

### Example 1: GDF-9 Western Blot Analysis

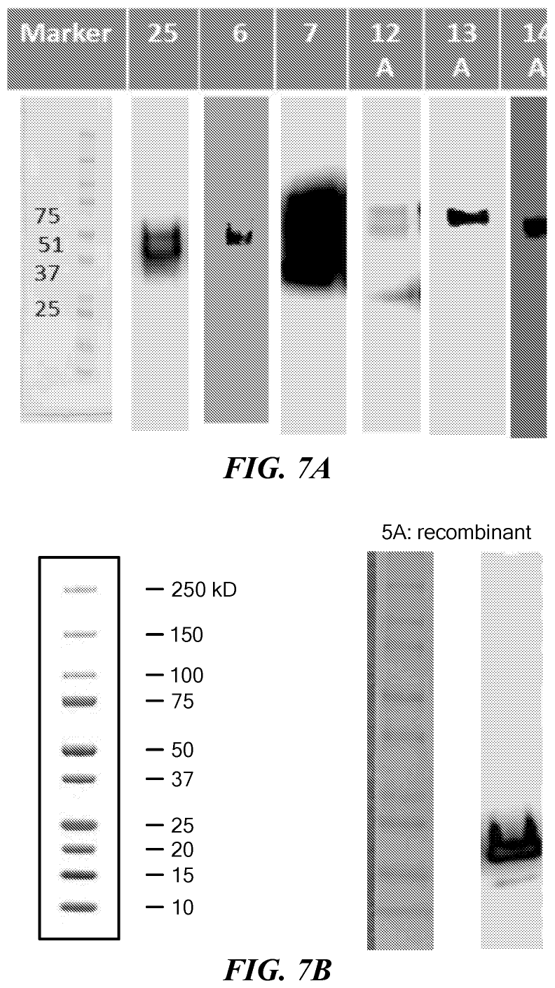

Western blot analysis was carried out using several anti-GDF-9 antibodies (see FIGS. 7A and 7B, and Table 1 below). These antibodies were found to recognize the 16 kDa monomer (FIG. 7B) and the 54 kDa pro-mature complex (FIG. 7A).

TABLE 1

| GDF-9 | | | | | | | | |
|---|---|---|---|---|---|---|---|---|
| # | # | | hGDF9 | hGDF-9 | hGDF-9 | | IHC | IHC |
| CLONE | In-house | LOT | 020614 | FL | Mature | Binding | Human | Mouse |
| 98-74A | 6 | O12215 | +++ | +++ | TBD | Mature | | +++ |
| 98-68A | 7 | O12215 | +++ | +++ | +++ | Mature | ++++ | ++++ |
| 114-5A | 12A | 102115 | +++ | +++ | +++ | Mature | | +++ |
| 114-31A | 14A | 101515 | +++ | +++ | +++ | Mature | | +++ |
| 99-69A | 25 | O20315 | +++ | +++ | TBD | Pro | | |
| 114-29A | 13A | 100815 | +++ | +++ | +++ | Mature | | |
| 114-58A | 17A | 102115 | TBD | TBD | TBD | Mature | | |
| 114-62 | 10A | 101615 | TBD | TBD | TBD | | | ++++ |

### Example 2: Epitope Mapping of Anti-GDF-9 Antibodies

Epitopes of newly generated anti-GDF-9 antibodies were determined using overlapping synthetic peptides corresponding to the entire amino acid sequence of GDF-9. Each peptide includes a tag of four amino acids (SGSG) at its N-terminus. Amino acid sequences of the overlapping peptides, SEQ ID NOS. of the epitopes, and results of binding of the anti-GDF-9 antibodies to the various peptides are shown in Tables 2.1, 2.2 and 2.3, respectively, below.

TABLE 2.1

| Peptide | | | |
|---|---|---|---|
| No | N-Term | Sequence | C-Term |
| 78 | Biotin- | SGSGMARPNKFLLWFCCFA | —NH2 |
| 79 | Biotin- | SGSGKFLLWFCCFAWLCFP | —NH2 |
| 80 | Biotin- | SGSGFCCFAWLCFPISLGS | —NH2 |
| 81 | Biotin- | SGSGWLCFPISLGSQASGG | —NH2 |
| 82 | Biotin- | SGSGISLGSQASGGEAQIA | —NH2 |
| 83 | Biotin- | SGSGQASGGEAQIAASAEL | —NH2 |
| 84 | Biotin- | SGSGEAQIAASAELESGAM | —NH2 |
| 85 | Biotin- | SGSGASAELESGAMPWSLL | —NH2 |
| 86 | Biotin- | SGSGESGAMPWSLLQHIDE | —NH2 |
| 87 | Biotin- | SGSGPWSLLQHIDERDRAG | —NH2 |
| 88 | Biotin- | SGSGQHIDERDRAGLLPAL | —NH2 |
| 89 | Biotin- | SGSGRDRAGLLPALFKVLS | —NH2 |
| 90 | Biotin- | SGSGLLPALFKVLSVGRGG | —NH2 |
| 91 | Biotin- | SGSGFKVLSVGRGGSPRLQ | —NH2 |
| 92 | Biotin- | SGSGVGRGGSPRLQPDSRA | —NH2 |
| 93 | Biotin- | SGSGSPRLQPDSRALHYMK | —NH2 |
| 94 | Biotin- | SGSGPDSRALHYMKKLYKT | —NH2 |
| 95 | Biotin- | SGSGLHYMKKLYKTYATKE | —NH2 |
| 96 | Biotin- | SGSGKLYKTYATKEGIPKS | —NH2 |
| 97 | Biotin- | SGSGYATKEGIPKSNRSHL | —NH2 |
| 98 | Biotin- | SGSGGIPKSNRSHLYNTVR | —NH2 |
| 99 | Biotin- | SGSGNRSHLYNTVRLFTPC | —NH2 |
| 100 | Biotin- | SGSGYNTVRLFTPCTRHKQ | —NH2 |
| 101 | Biotin- | SGSGLFTPCTRHKQAPGDQ | —NH2 |
| 102 | Biotin- | SGSGTRHKQAPGDQVTGIL | —NH2 |
| 103 | Biotin- | SGSGAPGDQVTGILPSVEL | —NH2 |
| 104 | Biotin- | SGSGVTGILPSVELLFNLD | —NH2 |
| 105 | Biotin- | SGSGPSVELLFNLDRITTV | —NH2 |
| 106 | Biotin- | SGSGLFNLDRITTVEHLLK | —NH2 |
| 107 | Biotin- | SGSGRITTVEHLLKSVLLY | —NH2 |
| 108 | Biotin- | SGSGEHLLKSVLLYNINNS | —NH2 |
| 109 | Biotin- | SGSGSVLLYNINNSVSFSS | —NH2 |
| 110 | Biotin- | SGSGNINNSVSFSSAVKCV | —NH2 |
| 111 | Biotin- | SGSGVSFSSAVKCVCNLMI | —NH2 |
| 112 | Biotin- | SGSGAVKCVCNLMIKEPKS | —NH2 |
| 113 | Biotin- | SGSGCNLMIKEPKSSSRTL | —NH2 |
| 114 | Biotin- | SGSGKEPKSSSRTLGRAPY | —NH2 |
| 115 | Biotin- | SGSGSSRTLGRAPYSFTFN | —NH2 |
| 116 | Biotin- | SGSGGRAPYSFTFNSQFEF | —NH2 |
| 117 | Biotin- | SGSGSFTFNSQFEFGKKHK | —NH2 |
| 118 | Biotin- | SGSGSQFEFGKKHKWIQID | —NH2 |
| 119 | Biotin- | SGSGGKKHKWIQIDVTSLL | —NH2 |
| 120 | Biotin- | SGSGWIQIDVTSLLQPLVA | —NH2 |
| 121 | Biotin- | SGSGVTSLLQPLVASNKRS | —NH2 |
| 122 | Biotin- | SGSGQPLVASNKRSIHMSI | —NH2 |
| 123 | Biotin- | SGSGSNKRSIHMSINFTCM | —NH2 |
| 124 | Biotin- | SGSGIHMSINFTCMKDQLE | —NH2 |
| 125 | Biotin- | SGSGNFTCMKDQLEHPSAQ | —NH2 |
| 126 | Biotin- | SGSGKDQLEHPSAQNGLFN | —NH2 |
| 127 | Biotin- | SGSGHPSAQNGLFNMTLVS | —NH2 |
| 128 | Biotin- | SGSGNGLFNMTLVSPSLIL | —NH2 |
| 129 | Biotin- | SGSGMTLVSPSLILYLNDT | —NH2 |
| 130 | Biotin- | SGSGPSLILYLNDTSAQAY | —NH2 |
| 131 | Biotin- | SGSGYLNDTSAQAYHSWYS | —NH2 |
| 132 | Biotin- | SGSGSAQAYHSWYSLHYKR | —NH2 |
| 133 | Biotin- | SGSGHSWYSLHYKRRPSQG | —NH2 |
| 134 | Biotin- | SGSGLHYKRRPSQGPDQER | —NH2 |
| 135 | Biotin- | SGSGRPSQGPDQERSLSAY | —NH2 |
| 136 | Biotin- | SGSGPDQERSLSAYPVGEE | —NH2 |
| 137 | Biotin- | SGSGSLSAYPVGEEAAEDG | —NH2 |
| 138 | Biotin- | SGSGPVGEEAAEDGRSSHH | —NH2 |
| 139 | Biotin- | SGSGAAEDGRSSHHRHRRG | —NH2 |
| 140 | Biotin- | SGSGRSSHHRHRRGQETVS | —NH2 |
| 141 | Biotin- | SGSGRHRRGQETVSSELKK | —NH2 |
| 142 | Biotin- | SGSGQETVSSELKKPLGPA | —NH2 |
| 143 | Biotin- | SGSGSELKKPLGPASFNLS | —NH2 |
| 144 | Biotin- | SGSGPLGPASFNLSEYFRQ | —NH2 |
| 145 | Biotin- | SGSGSFNLSEYFRQFLLPQ | —NH2 |
| 146 | Biotin- | SGSGEYFRQFLLPQNECEL | —NH2 |
| 147 | Biotin- | SGSGFLLPQNECELHDFRL | —NH2 |
| 148 | Biotin- | SGSGNECELHDFRLSFSQL | —NH2 |
| 149 | Biotin- | SGSGHDFRLSFSQLKWDNW | —NH2 |
| 150 | Biotin- | SGSGSFSQLKWDNWIVAPH | —NH2 |
| 151 | Biotin- | SGSGKWDNWIVAPHRYNPR | —NH2 |
| 152 | Biotin- | SGSGIVAPHRYNPRYCKGD | —NH2 |
| 153 | Biotin- | SGSGRYNPRYCKGDCPRAV | —NH2 |
| 154 | Biotin- | SGSGYCKGDCPRAVGHRYG | —NH2 |
| 155 | Biotin- | SGSGCPRAVGHRYGSPVHT | —NH2 |
| 156 | Biotin- | SGSGGHRYGSPVHTMVQNI | —NH2 |
| 157 | Biotin- | SGSGSPVHTMVQNIIYEKL | —NH2 |
| 158 | Biotin- | SGSGMVQNIIYEKLDSSVP | —NH2 |
| 159 | Biotin- | SGSGIYEKLDSSVPRPSCV | —NH2 |
| 160 | Biotin- | SGSGDSSVPRPSCVPAKYS | —NH2 |
| 161 | Biotin- | SGSGRPSCVPAKYSPLSVL | —NH2 |
| 162 | Biotin- | SGSGPAKYSPLSVLTIEPD | —NH2 |
| 163 | Biotin- | SGSGPLSVLTIEPDGSIAY | —NH2 |
| 164 | Biotin- | SGSGTIEPDGSIAYKEYED | —NH2 |
| 165 | Biotin- | SGSGGSIAYKEYEDMIATK | —NH2 |
| 166 | Biotin- | SGSGYKEYEDMIATKCTCR | —NH2 |

TABLE 2.2

| SEQ ID | |
|---|---|
| NO: | Peptide Sequence |
| 78 | MARPNKFLLWFCCFA |
| 79 | KFLLWFCCFAWLCFP |
| 80 | FCCFAWLCFPISLGS |
| 81 | WLCFPISLGSQASGG |
| 82 | ISLGSQASGGEAQIA |
| 83 | QASGGEAQIAASAEL |
| 84 | EAQIAASAELESGAM |
| 85 | ASAELESGAMPWSLL |
| 86 | ESGAMPWSLLQHIDE |
| 87 | PWSLLQHIDERDRAG |
| 88 | QHIDERDRAGLLPAL |
| 89 | RDRAGLLPALFKVLS |
| 90 | LLPALFKVLSVGRGG |
| 91 | FKVLSVGRGGSPRLQ |
| 92 | VGRGGSPRLQPDSRA |
| 93 | SPRLQPDSRALHYMK |
| 94 | PDSRALHYMKKLYKT |
| 95 | LHYMKKLYKTYATKE |
| 96 | KLYKTYATKEGIPKS |
| 97 | YATKEGIPKSNRSHL |
| 98 | GIPKSNRSHLYNTVR |
| 99 | NRSHLYNTVRLFTPC |
| 100 | YNTVRLFTPCTRHKQ |
| 101 | LFTPCTRHKQAPGDQ |
| 102 | TRHKQAPGDQVTGIL |
| 103 | APGDQVTGILPSVEL |
| 104 | VTGILPSVELLFNLD |
| 105 | PSVELLFNLDRITTV |
| 106 | LFNLDRITTVEHLLK |
| 107 | RITTVEHLLKSVLLY |
| 108 | EHLLKSVLLYNINNS |
| 109 | SVLLYNINNSVSFSS |
| 110 | NINNSVSFSSAVKCV |
| 111 | VSFSSAVKCVCNLMI |
| 112 | AVKCVCNLMIKEPKS |
| 113 | CNLMIKEPKSSSRTL |
| 114 | KEPKSSSRTLGRAPY |
| 115 | SSRTLGRAPYSFTFN |
| 116 | GRAPYSFTFNSQFEF |
| 117 | SFTFNSQFEFGKKHK |
| 118 | SQFEFGKKHKWIQID |
| 119 | GKKHKWIQIDVTSLL |
| 120 | WIQIDVTSLLQPLVA |
| 121 | VTSLLQPLVASNKRS |
| 122 | QPLVASNKRSIHMSI |
| 123 | SNKRSIHMSINFTCM |
| 124 | IHMSINFTCMKDQLE |
| 125 | NFTCMKDQLEHPSAQ |
| 126 | KDQLEHPSAQNGLFN |
| 127 | HPSAQNGLFNMTLVS |
| 128 | NGLFNMTLVSPSLIL |
| 129 | MTLVSPSLILYLNDT |
| 130 | PSLILYLNDTSAQAY |
| 131 | YLNDTSAQAYHSWYS |
| 132 | SAQAYHSWYSLHYKR |
| 133 | HSWYSLHYKRRPSQG |
| 134 | LHYKRRPSQGPDQER |
| 135 | RPSQGPDQERSLSAY |
| 136 | PDQERSLSAYPVGEE |
| 137 | SLSAYPVGEEAAEDG |
| 138 | PVGEEAAEDGRSSHH |
| 139 | AAEDGRSSHHRHRRG |
| 140 | RSSHHRHRRGQETVS |
| 141 | RHRRGQETVSSELKK |
| 142 | QETVSSELKKPLGPA |
| 143 | SELKKPLGPASFNLS |
| 144 | PLGPASFNLSEYFRQ |
| 145 | SFNLSEYFRQFLLPQ |
| 146 | EYFRQFLLPQNECEL |
| 147 | FLLPQNECELHDFRL |
| 148 | NECELHDFRLSFSQL |
| 149 | HDFRLSFSQLKWDNW |
| 150 | SFSQLKWDNWIVAPH |
| 151 | KWDNWIVAPHRYNPR |
| 152 | IVAPHRYNPRYCKGD |
| 153 | RYNPRYCKGDCPRAV |
| 154 | YCKGDCPRAVGHRYG |
| 155 | CPRAVGHRYGSPVHT |
| 156 | GHRYGSPVHTMVQNI |
| 157 | SPVHTMVQNIIYEKL |
| 158 | MVQNIIYEKLDSSVP |
| 159 | IYEKLDSSVPRPSCV |
| 160 | DSSVPRPSCVPAKYS |
| 161 | RPSCVPAKYSPLSVL |
| 162 | PAKYSPLSVLTIEPD |
| 163 | PLSVLTIEPDGSIAY |
| 164 | TIEPDGSIAYKEYED |
| 165 | GSIAYKEYEDMIATK |
| 166 | YKEYEDMIATKCTCR |

TABLE 2.3

| | | | Binding epitope | | Binding epitope | | Binding epitope | | Binding epitope |
|---|---|---|---|---|---|---|---|---|---|
| | Clone | Immunogen | (peptide number) | | (peptide number) | | (peptide number) | | (peptide number) |
| 1 | 98/20A | GDF-9 (P + M) | 123 | (++) | 130-134 | (+++) | | | |
| 2 | 98/62A | GDF-9 (P + M) | 123 | (++) | 132 | (+++) | | | |
| 3 | 98/80A | GDF-9 (P + M) | 123-124 | (+) | 132-133 | (+++) | | | |
| 4 | 98/5A | GDF-9 (P + M) | 123 | (+++) | 132-133 | (+++) | | | |
| 5 | 98/58A | GOF-9 (P + M) | 123-125 | (++) | 132 | (+++) | | | |
| 6 | 98/74A | GDF-9 (P + M) | 123 | (++) | 129 | (++) | 132 | (++) | 144-146 (+++) |
| 7 | 98/68A | GDF-9 (P + M) | NA | | | | | | |
| 11 | 98/30A | GDF-9 (P + M) | 123 | (++) | 129 | (+) | 132-133 | (+++) | |
| 13 | 98/83A | GDF-9 (P + M) | 119-120 | (++) | 125 | (+) | 128-129 | (+++) | |
| 14 | 98/37A | GDF-9 (P + M) | 111 | (+) | 123 | (+++) | 132-134 | (+++) | |
| 17 | 99/12A | GDF-9 (proFc) | NA | | | | | | |
| 21 | 99/90A | GDF-9 (proFc) | NA | | | | | | |
| 25 | 99/69A | GDF-9 (proFc) | NA | | | | | | |
| 10A | 114/62A | BMP-15 + GDF-9 | NA | | | | | | |
| 12A | 114/5A | BMP-15 + GDF-9 | NA | | | | | | |
| 13A | 114/29A | BMP-15 + GDF-9 | 111 | (+) | 123 | (+++) | 132 | (+) | |

| 14A | 114/31A | BMP-15 + GDF-9 | NA | | | |
|---|---|---|---|---|---|---|
| 16A | 114/50A | BMP-15 + GDF-9 | NA | | | |
| 17A | 114/58A | BMP-15 + GDF-9 | 123 | (+) | 135, 136 | (+++) |

### Example 3: BMP-15 Western Blot Analysis

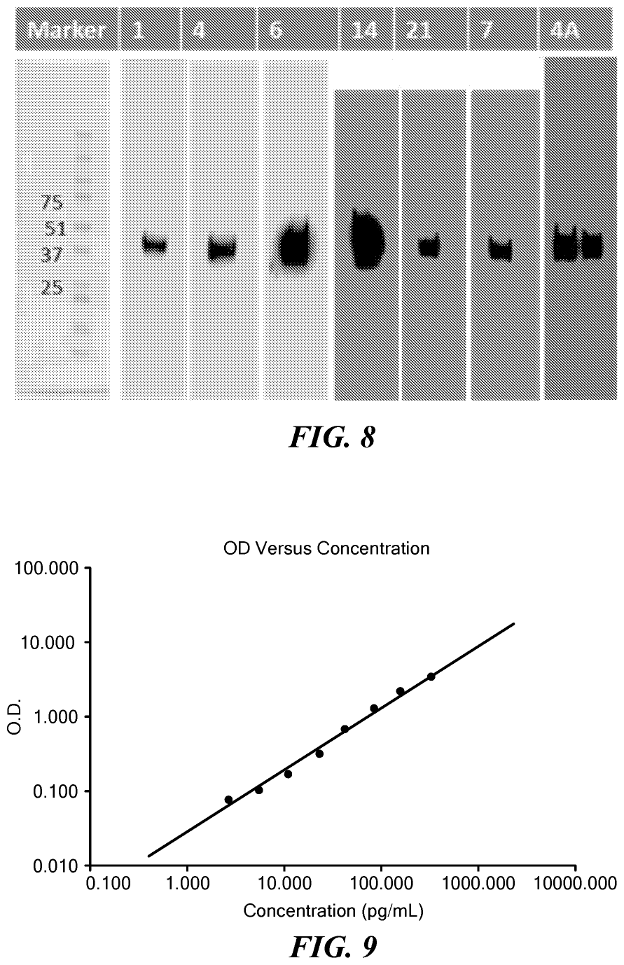

Western blot analysis was carried out using several anti-BMP-15 antibodies (see FIG. 8, and Table 3 below). These antibodies were found to recognize the 37 kDa pro-mature complex of BMP-15.

TABLE 3

| | | CHO cells Antigen | | | |
|---|---|---|---|---|---|
| BMP15 | | hBMP15 | hBMP15 FL | Binding | IHC |
| CLONE # | Ansh # | 161213 | U3513 | location | Mouse |
| 114-1A | 1 | +++ | +++ | Pro + Mature | |
| 87-38A | 4 | +++ | +++ | Pro + Mature | |
| 87-63A | 6 | +++ | +++ | Pro + Mature | |
| 94-29A | 14 | +++ | +++ | Pro + Mature | |
| 111-39A | 21 | +++ | +++ | Pro + Mature | |
| 114-35A | 7 | +++ | +++ | Pro + Mature | |
| 114-16A | 4A | +++ | +++ | Pro + Mature | |
| 111-1A | 20 | | | | ++++ |
| 111-77A | 19 | | | | ++++ |
| 111-49A | 22 | | | | +++ |
| 111-53A | 23 | | | | +++ |
| | 23A | | | Mature | |

Example 4: Epitope Mapping of Anti-BMP-15 Antibodies

Epitopes bound by newly generated anti-BMP-15 antibodies were determined using overlapping synthetic peptides corresponding to the entire amino acid sequence of BMP-15. Each peptide includes a tag of four amino acids (SGSG) at the N-terminus. Amino acid sequences of the overlapping peptides, SEQ ID NOS. of the epitopes, and results of binding of the anti-BMP-15 antibodies to the various peptides are shown in Tables 4.1, 4.2 and 4.3, respectively, below.

TABLE 4.1

| Peptide | | | | | |
|---|---|---|---|---|---|
| No | N-Term | Sequence | C-Term | aa start | aa finish |
| 1 | Biotin- | SGSGMVLLSILRILFLCEL | —NH2 | 1 | 15 |
| 2 | Biotin- | SGSGILRILFLCELVLFME | —NH2 | 5 | 20 |
| 3 | Biotin- | SGSGFLCELVLFMEHRAQM | —NH2 | 10 | 25 |
| 4 | Biotin- | SGSGVLFMEHRAQMAEGGQ | —NH2 | 15 | 30 |
| 5 | Biotin- | SGSGHRAQMAEGGQSSIAL | —NH2 | 20 | 35 |
| 6 | Biotin- | SGSGAEGGQSSIALLAEAP | —NH2 | 25 | 40 |
| 7 | Biotin- | SGSGSSIALLAEAPTLPLI | —NH2 | 30 | 45 |
| 8 | Biotin- | SGSGLAEAPTLPLIEELLE | —NH2 | 35 | 50 |
| 9 | Biotin- | SGSGTLPLIEELLEESPGE | —NH2 | 40 | 55 |
| 10 | Biotin- | SGSGEELLEESPGEQPRKP | —NH2 | 45 | 60 |
| 11 | Biotin- | SGSGESPGEQPRKPRLLGH | —NH2 | 50 | 65 |
| 12 | Biotin- | SGSGQPRKPRLLGHSLRYM | —NH2 | 55 | 70 |
| 13 | Biotin- | SGSGRLLGHSLRYMLELYR | —NH2 | 60 | 75 |
| 14 | Biotin- | SGSGSLRYMLELYRRSADS | —NH2 | 65 | 80 |
| 15 | Biotin- | SGSGLELYRRSADSHGHPR | —NH2 | 70 | 85 |
| 16 | Biotin- | SGSGRSADSHGHPRENRTI | —NH2 | 75 | 90 |
| 17 | Biotin- | SGSGHGHPRENRTIGATMV | —NH2 | 80 | 95 |
| 18 | Biotin- | SGSGENRTIGATMVRLVKP | —NH2 | 85 | 100 |
| 19 | Biotin- | SGSGGATMVRLVKPLTNVA | —NH2 | 90 | 105 |
| 20 | Biotin- | SGSGRLVKPLTNVARPHRG | —NH2 | 95 | 110 |
| 21 | Biotin- | SGSGLTNVARPHRGTWHIQ | —NH2 | 100 | 115 |
| 22 | Biotin- | SGSGRPHRGTWHIQILGFP | —NH2 | 105 | 120 |
| 23 | Biotin- | SGSGTWHIQILGFPLRPNR | —NH2 | 110 | 125 |
| 24 | Biotin- | SGSGILGFPLRPNRGLYQL | —NH2 | 115 | 130 |
| 25 | Biotin- | SGSGLRPNRGLYQLVRATV | —NH2 | 120 | 135 |
| 26 | Biotin- | SGSGGLYQLVRATVVYRHH | —NH2 | 125 | 140 |
| 27 | Biotin- | SGSGVRATVVYRHHLQLTR | —NH2 | 130 | 145 |
| 28 | Biotin- | SGSGVYRHHLQLTRFNLSC | —NH2 | 135 | 150 |
| 29 | Biotin- | SGSGLQLTRFNLSCHVEPW | —NH2 | 140 | 155 |
| 30 | Biotin- | SGSGFNLSCHVEPWVQKNP | —NH2 | 145 | 160 |
| 31 | Biotin- | SGSGHVEPWVQKNPTNHFP | —NH2 | 150 | 165 |
| 32 | Biotin- | SGSGVQKNPTNHFPSSEGD | —NH2 | 155 | 170 |
| 33 | Biotin- | SGSGTNHFPSSEGDSSKPS | —NH2 | 160 | 175 |
| 34 | Biotin- | SGSGSSEGDSSKPSLMSNA | —NH2 | 165 | 180 |
| 35 | Biotin- | SGSGSSKPSLMSNAWKEMD | —NH2 | 170 | 185 |
| 36 | Biotin- | SGSGLMSNAWKEMDITQLV | —NH2 | 175 | 190 |
| 37 | Biotin- | SGSGWKEMDITQLVQQRFW | —NH2 | 180 | 195 |
| 38 | Biotin- | SGSGITQLVQQRFWNNKGH | —NH2 | 185 | 200 |
| 39 | Biotin- | SGSGQQRFWNNKGHRILRL | —NH2 | 190 | 205 |
| 40 | Biotin- | SGSGNNKGHRILRLRFMCQ | —NH2 | 195 | 210 |
| 41 | Biotin- | SGSGRILRLRFMCQQQKDS | —NH2 | 200 | 215 |
| 42 | Biotin- | SGSGRFMCQQQKDSGGLEL | —NH2 | 205 | 220 |
| 43 | Biotin- | SGSGQQKDSGGLELWHGTS | —NH2 | 210 | 225 |
| 44 | Biotin- | SGSGGGLELWHGTSSLDIA | —NH2 | 215 | 230 |
| 45 | Biotin- | SGSGWHGTSSLDIAFLLLY | —NH2 | 220 | 235 |
| 46 | Biotin- | SGSGSLDIAELLLYENDTH | —NH2 | 225 | 240 |
| 47 | Biotin- | SGSGELLLYENDTHKSIRK | —NH2 | 230 | 245 |
| 48 | Biotin- | SGSGENDTHKSIRKAKFLP | —NH2 | 235 | 250 |
| 49 | Biotin- | SGSGKSIRKAKFLPRGMEE | —NH2 | 240 | 255 |
| 50 | Biotin- | SGSGAKFLPRGMEEFMERE | —NH2 | 245 | 260 |
| 51 | Biotin- | SGSGRGMEEFMERESLLRR | —NH2 | 250 | 265 |
| 52 | Biotin- | SGSGFMERESLLRRTRQAD | —NH2 | 255 | 270 |
| 53 | Biotin- | SGSGSLLRRTRQADGISAE | —NH2 | 260 | 275 |
| 54 | Biotin- | SGSGTRQADGISAEVTASS | —NH2 | 265 | 280 |
| 55 | Biotin- | SGSGGISAEVTASSSKHSG | —NH2 | 270 | 285 |
| 56 | Biotin- | SGSGVTASSSKHSGPENNQ | —NH2 | 275 | 290 |
| 57 | Biotin- | SGSGSKHSGPENNQCSLHP | —NH2 | 280 | 295 |
| 58 | Biotin- | SGSGPENNQCSLHPFQISF | —NH2 | 285 | 300 |
| 59 | Biotin- | SGSGCSLHPFQISFRQLGW | —NH2 | 290 | 305 |
| 60 | Biotin- | SGSGFQISFRQLGWDHWII | —NH2 | 295 | 310 |
| 61 | Biotin- | SGSGRQLGWDHWIIAPPFY | —NH2 | 300 | 315 |
| 62 | Biotin- | SGSGDHWIIAPPFYTPNYC | —NH2 | 305 | 320 |
| 63 | Biotin- | SGSGAPPFYTPNYCKGTCL | —NH2 | 310 | 325 |
| 64 | Biotin- | SGSGTPNYCKGTCLRVLRD | —NH2 | 315 | 330 |
| 65 | Biotin- | SGSGKGTCLRVLRDGLNSP | —NH2 | 320 | 335 |
| 66 | Biotin- | SGSGRVLRDGLNSPNHAII | —NH2 | 325 | 340 |
| 67 | Biotin- | SGSGGLNSPNHAIIQNLIN | —NH2 | 330 | 345 |
| 68 | Biotin- | SGSGNHAIIQNLINQLVDQ | —NH2 | 335 | 350 |
| 69 | Biotin- | SGSGQNLINQLVDQSVPRP | —NH2 | 340 | 355 |
| 70 | Biotin- | SGSGQLVDQSVPRPSCVPY | —NH2 | 345 | 360 |
| 71 | Biotin- | SGSGSVPRPSCVPYKYVPI | —NH2 | 350 | 365 |
| 72 | Biotin- | SGSGSCVPYKYVPISVLMI | —NH2 | 355 | 370 |
| 73 | Biotin- | SGSGKYVPISVLMIEANGS | —NH2 | 360 | 375 |
| 74 | Biotin- | SGSGSVLMIEANGSILYKE | —NH2 | 365 | 380 |
| 75 | Biotin- | SGSGEANGSILYKEYEGMI | —NH2 | 370 | 385 |
| 76 | Biotin- | SGSGILYKEYEGMIAESCT | —NH2 | 375 | 390 |
| 77 | Biotin- | SGSGYKEYEGMIAESCTCR | —NH2 | 380 | 395 |

TABLE 4.2

| SEQ ID NO: | Peptide Sequence | aa start | aa finish |
|---|---|---|---|
| 1 | MVLLSILRILFLCEL | 1 | 15 |
| 2 | ILRILFLCELVLFME | 5 | 20 |
| 3 | FLCELVLFMEHRAQM | 10 | 25 |
| 4 | VLFMEHRAQMAEGGQ | 15 | 30 |
| 5 | HRAQMAEGGQSSIAL | 20 | 35 |
| 6 | AEGGQSSIALLAEAP | 25 | 40 |
| 7 | SSIALLAEAPTLPLI | 30 | 45 |
| 8 | LAEAPTLPLIEELLE | 35 | 50 |
| 9 | TLPLIEELLEESPGE | 40 | 55 |
| 10 | EELLEESPGEQPRKP | 45 | 60 |
| 11 | ESPGEQPRKPRLLGH | 50 | 65 |
| 12 | QPRKPRLLGHSLRYM | 55 | 70 |
| 13 | RLLGHSLRYMLELYR | 60 | 75 |
| 14 | SLRYMLELYRRSADS | 65 | 80 |
| 15 | LELYRRSADSHGHPR | 70 | 85 |
| 16 | RSADSHGHPRENRTI | 75 | 90 |
| 17 | HGHPRENRTIGATMV | 80 | 95 |
| 18 | ENRTIGATMVRLVKP | 85 | 100 |
| 19 | GATMVRLVKPLTNVA | 90 | 105 |
| 20 | RLVKPLTNVARPHRG | 95 | 110 |
| 21 | LTNVARPHRGTWHIQ | 100 | 115 |
| 22 | RPHRGTWHIQILGFP | 105 | 120 |
| 23 | TWHIQILGFPLRPNR | 110 | 125 |
| 24 | ILGFPLRPNRGLYQL | 115 | 130 |
| 25 | LRPNRGLYQLVRATV | 120 | 135 |
| 26 | GLYQLVRATVVYRHH | 125 | 140 |
| 27 | VRATVVYRHHLQLTR | 130 | 145 |
| 28 | VYRHHLQLTRFNLSC | 135 | 150 |
| 29 | LQLTRFNLSCHVEPW | 140 | 155 |
| 30 | FNLSCHVEPWVQKNP | 145 | 160 |
| 31 | HVEPWVQKNPTNHFP | 150 | 165 |
| 32 | VQKNPTNHFPSSEGD | 155 | 170 |
| 33 | TNHFPSSEGDSSKPS | 160 | 175 |
| 34 | SSEGDSSKPSLMSNA | 165 | 180 |
| 35 | SSKPSLMSNAWKEMD | 170 | 185 |
| 36 | LMSNAWKEMDITQLV | 175 | 190 |
| 37 | WKEMDITQLVQQRFW | 180 | 195 |
| 38 | ITQLVQQRFWNNKGH | 185 | 200 |
| 39 | QQRFWNNKGHRILRL | 190 | 205 |
| 40 | NNKGHRILRLRFMCQ | 195 | 210 |
| 41 | RILRLRFMCQQQKDS | 200 | 215 |
| 42 | RFMCQQQKDSGGLEL | 205 | 220 |
| 43 | QQKDSGGLELWHGTS | 210 | 225 |
| 44 | GGLELWHGTSSLDIA | 215 | 230 |
| 45 | WHGTSSLDIAFLLLY | 220 | 235 |
| 46 | SLDIAELLLYENDTH | 225 | 240 |
| 47 | FLLLYENDTHKSIRK | 230 | 245 |
| 48 | FNDTHKSIRKAKFLP | 235 | 250 |
| 49 | KSIRKAKFLPRGMEE | 240 | 255 |
| 50 | AKFLPRGMEEFMERE | 245 | 260 |
| 51 | RGMEEFMERESLLRR | 250 | 265 |
| 52 | FMERESLLRRTRQAD | 255 | 270 |
| 53 | SLLRRTRQADGISAE | 260 | 275 |
| 54 | TRQADGISAEVTASS | 265 | 280 |
| 55 | GISAEVTASSSKHSG | 270 | 285 |
| 56 | VTASSSKHSGPENNQ | 275 | 290 |
| 57 | SKHSGPENNQCSLHP | 280 | 295 |
| 58 | PENNQCSLHPFQISF | 285 | 300 |
| 59 | CSLHPFQISFRQLGW | 290 | 305 |
| 60 | FQISFRQLGWDHWII | 295 | 310 |
| 61 | RQLGWDHWIIAPPFY | 300 | 315 |
| 62 | DHWIIAPPFYTPNYC | 305 | 320 |
| 63 | APPFYTPNYCKGTCL | 310 | 325 |
| 64 | TPNYCKGTCLRVLRD | 315 | 330 |
| 65 | KGTCLRVLRDGLNSP | 320 | 335 |
| 66 | RVLRDGLNSPNHAII | 325 | 340 |
| 67 | GLNSPNHAIIQNLIN | 330 | 345 |
| 68 | NHAIIQNLINQLVDQ | 335 | 350 |
| 69 | QNLINQLVDQSVPRP | 340 | 355 |
| 70 | QLVDQSVPRPSCVPY | 345 | 360 |
| 71 | SVPRPSCVPYKYVPI | 350 | 365 |
| 72 | SCVPYKYVPISVLMI | 355 | 370 |
| 73 | KYVPISVLMIEANGS | 360 | 375 |
| 74 | SVLMIEANGSILYKE | 365 | 380 |
| 75 | EANGSILYKEYEGMI | 370 | 385 |
| 76 | ILYKEYEGMIAESCT | 375 | 390 |
| 77 | YKEYEGMIAESCTCR | 380 | 395 |

TABLE 4.3

| 1 | 87/7A | BMP-15 (P + M) | 41 | (+) | 50-51 | (++) | | | | | | |
|---|---|---|---|---|---|---|---|---|---|---|---|---|
| 2 | 87/30A | BMP-15 (P + M) | multiple epitopes | | | | | | | | | |
| 3 | 87/33A | BMP-15 (P + M) | 8-9 | (+++) | 12 | (+) | | | | | | |
| 4 | 87/38A | BMP-15 (P + M) | 8-9 | (+++) | 48-50 | (+) | | | | | | |
| 5 | 87/56A | BMP-15 (P + M) | non specific | | | | | | | | | |
| 6 | 87/63A | BMP-15 (P + M) | 8 | (++) | 10-11 | (+) | 28 | (+) | 40 | (+) | 49-50 | (+) |
| 7 | 87/82B | BMP-15 (P + M) | 8 | (+) | 49-50 | (++) | | | | | | |
| 8 | 87/87A | BMP-15 (P + M) | 8-9 | (+++) | | | | | | | | |
| 9 | 87/1A | BMP-15 (P + M) | non specific | | | | | | | | | |
| 10 | 87/13A | BMP-15 (P + M) | non specific | | | | | | | | | |
| 11 | 87/18A | BMP-15 (P + M) | 10 | (+) | 40 | (+) | 49-50 | (+) | | | | |
| 12 | 87/55A | BMP-15 (P + M) | 8 | (+) | 40 | (+) | 49-50 | (++) | | | | |
| 13 | 94/20A | BMP-15 (P + M) | non specific | | | | | | | | | |
| 14 | 94/29A | BMP-15 (P + M) | non specific | | | | | | | | | |
| 15 | 94/34A | BMP-15 (P + M) | 8, 28, 30, 40, | | | | | | | | | |
| | | | 46, 68 (+) | | | | | | | | | |
| 16 | 94/38A | BMP-15 (P + M) | 8-11 | (++) | 28 | (+) | 40 | (+) | 68 | (+) | | |
| 19 | 111/77A | BMP-15 | 8-9 | (+++) | 45-46 | (+) | | | | | | |
| | | (BMP-15/GDF KO) | | | | | | | | | | |
| 20 | 111/1A | BMP-15 | 32-33 | (++) | | | | | | | | |
| | | (BMP-15/GDF KO) | | | | | | | | | | |
| 21 | 111/39A | BMP-15 | 8-11 | (+++) | 32 | (+) | 45-46 | (+) | | | | |
| | | (BMP-15/GDF KO) | | | | | | | | | | |
| 22 | 111/49A | BMP-15 | 8-9 | (+++) | 32-33 | (+++) | 46 | (+) | 61 | (+) | 68 | (+) |
| | | (BMP-15/GDF KO) | | | | | | | | | | |
| 23 | 111/53A | BMP-15 | 32-33 | (+++) | 46 | (+++) | 68 | (+++) | | | | |
| | | (BMP-15/GDF KO) | | | | | | | | | | |
| 25 | 111/65A | BMP-15 | 8-9 | (+++) | 32-33 | (+++) | 46 | (+++) | 61 | (+) | 68 | (+) |
| | | (BMP-15/GDF KO) | | | | | | | | | | |

| | | | | | | |
|---|---|---|---|---|---|---|
| 26 | 111/68A | BMP-15 | 32-34 | (+++) | 55 | (+) |
| | | (BMP-15/GDF KO) | | | | |
| 28 | 111/11A | BMP-15 | 8-9 | (+) | 32-33 | (+) |
| | | (BMP-15/GDF KO) | | | | |

| | | | |
|---|---|---|---|
| 1A | 114/1A | BMP-15 + GDF-9 | Non specific |
| | | (BMP-15/GDF KO) | |
| 4A | 114/16A | BMP-15 + GDF-9 | Non specific |
| | | (BMP-15/GDF KO) | |
| 7A | 114/35A | BMP-15 + GDF-9 | Non specific |
| | | (BMP-15/GDF KO) | |
| 8A | 114/40A | BMP-15 + GDF-9 | Non specific |
| | | (BMP-15/GDF KO) | |

### Example 5: ELISA with Anti-GDF-9 Capture and Anti-BMP-15 Detection Antibodies With and Without Dissociating Agent

ELISA was performed using an anti-GDF-9 antibody for capture and an anti-BMP-15 antibody for detection, both in the presence and in the absence of the dissociating agent SDS (see Table 3 below for details). Both animal and human follicular fluids were used as samples. Two different assays were performed, each using the same antibody for capture, but different antibodies for detection. The capture antibody was anti-GDF-9 monoclonal antibody 25A (clone 128/33A) which recognizes region of GDF-9. The detection antibodies were anti-BMP-15 monoclonal antibodies 5A (clone 114/24A), which recognizes region and 23A (clone 128/98A) which recognize the mature region of the protein. Results show that the detection of GDF-9-BMP-15 complex was not affected by the presence of SDS.

TABLE 5
50 μl sample + 50 μl ASB* --> 3 hr inc. --> 100 μl
5K biotin --> 90 min inc. ---> 100 μl 30K SHRP --> 30 min --> 100 μl RMB --> 10 min -->100 μl STOP

| | 1 | 2 | 1 | 2 | 3 | 4 | 5 | 6 | 7 | 8 |
|---|---|---|---|---|---|---|---|---|---|---|
| A | ACT A | Mouse Ovary | 0.022 | 0.014 | 0.017 | 0.017 | 0.017 | 0.016 | 0.025 | 0.026 |
| | Matrix | extract 1:10 | | | | | | | | |
| B | GDF-9 | Canine Extract | 0.018 | 0.031 | 0.018 | 0.032 | 0.015 | 0.03 | 0.027 | 0.069 |
| | p + m | 1:5 | | | | | | | | |
| | (1 ng/mL) | | | | | | | | | |
| C | BMP-15 | Bovine FF | 2.902 | 0.025 | 3.358 | 0.025 | 3.49 | 0.028 | 3.686 | 0.039 |
| | p + m | Neat | | | | | | | | |
| | (1 ng/mL) | | | | | | | | | |
| D | Complex | Equine FF | 0.675 | 0.024 | 0.761 | 0.026 | 0.961 | 0.027 | 0.963 | 0.042 |
| | 1:10,000 | neat | | | | | | | | |
| E | Complex | Human | 1.289 | 0.024 | 1.464 | 0.024 | 1.824 | 0.025 | 1.839 | 0.043 |
| | 1:5000 | FF-2 | | | | | | | | |
| F | Complex | Human | 2.673 | 0.021 | 2.965 | 0.022 | 3.486 | 0.025 | 3.642 | 0.043 |
| | 1:2000 | FF-A4 | | | | | | | | |
| G | Complex | Human | 3.687 | 0.019 | 3.907 | 0.025 | 4.011 | 0.021 | 3.702 | 0.033 |
| | 1:1000 | FF-A3 | | | | | | | | |
| H | Mouse Testes | Human | 0.043 | 0.024 | 0.04 | 0.028 | 0.049 | 0.027 | 0.068 | 0.042 |
| | extract 1:10 | FF-3 | | | | | | | | |

| | | | | |
|---|---|---|---|---|
| ASB | CND-123 | CND-123 + SDS | CND-123 | CND-123 + SDS |
| | | Buffer (1:1) | | Buffer (1:1) |

| Capture GDF-9 | 25A(128/33A) | |
|---|---|---|
| Detection BMP-15 | 5A(114/24A) | 23A(128/98A) |

### Example 6. ELISA with Anti-BMP-15 Capture and Anti-GDF-9 Detection Antibodies With and Without Dissociating Agent

ELISA was performed using an anti-BMP-15 antibody for capture and an anti-GDF-9 antibody for detection in the presence and absence of SDS as the dissociating agent (see Table 6 below for details). Both animal and human follicular fluids were used as samples for the assay. Two different assays were performed, each using the same antibody for capture but different antibodies for detection. The capture antibody was anti-BMP-15 monoclonal antibody 23A (clone 128/98A) which recognizes the mature region of the protein. The detection antibodies were anti-GDF-9 monoclonal antibodies 12A (clone 114/31A) and 25A (clone 128/79A). Results show that the detection of BMP-15-GDF-9 complex was not affected by the presence of SDS.

TABLE 6
50 μl sample + 50 μl ASB* --> 3 hr inc. --> 100 μl 5K
biotin --> 90 min inc. ---> 100 μl 30K SHRP --> 30
min --> 100 μl TMB --> 10 min --> 100 μl STOP

| Plate 1 | | | 1 | 2 | 3 | 4 | 5 | 6 | 7 | 8 |
|---|---|---|---|---|---|---|---|---|---|---|
| A | ACT A | Moose Ovary | 0.113 | 0.12 | 0.345 | 0.386 | 0.099 | 0.113 | 0.438 | 0.475 |
| | Matrix | extract 1:10 | | | | | | | | |
| B | GDF-9 | Canine extract | 0.113 | 0.134 | 0.35 | 0.497 | 0.101 | 0.144 | 0.466 | 0.595 |
| | p + m | 1:5 | | | | | | | | |
| | (1 ng/mL | | | | | | | | | |
| C | BMP-15 | Bovine FF | 2.911 | 0.119 | 2.823 | 0.229 | 3.751 | 0.118 | 3.95 | 0.259 |
| | p + m | Neat | | | | | | | | |
| | (1 ng/mL) | | | | | | | | | |
| D | Complex | Equine FF | 0.821 | 0.11 | 0.839 | 0.231 | 1.149 | 0.116 | 1.182 | 0.238 |
| | 1:10,000 | neat | | | | | | | | |
| E | Complex | Human | 1.501 | 0.115 | 1.357 | 0.218 | 2.15 | 0.106 | 1.764 | 0.214 |
| | 1:5000 | FF-2 | | | | | | | | |
| F | Complex | Human | 3.245 | 0.119 | 2.639 | 0.223 | 3.996 | 0.11 | 3.233 | 0.235 |
| | 1:2000 | FF-A4 | | | | | | | | |
| G | Complex | Human | 4.248 | 0.122 | 4.021 | 0.294 | 4.294 | 0.111 | 4.194 | 0.231 |
| | 1:1000 | FF-A3 | | | | | | | | |
| H | Mouse Testes | Human | 0.162 | 0.127 | 0.826 | 0.204 | 0.146 | 0.117 | 0.74 | 0.264 |
| | extract 1:10 | FF-3 | | | | | | | | |

| | | | | |
|---|---|---|---|---|
| ASB | CND-123 | CND-123 + | CND-123 | CND-123 + |
| | | ASB-123A (1:1) | | ASB-123A (1:1) |

| | | |
|---|---|---|
| Capture BMP-15 | 23A(128/98A) | 23A(128/98A) |
| Detection-GDF-9 | 12A(114/31A) | 25A(128/79A) |

### Example 7: Additional Assays for Detecting BMP-15, GDF-9, and a Complex Between BMP-15 and GDF-9

Several additional assays were performed for detecting one or more of BMP-15, GDF-9, or a complex between BMP-15 and GDF-9. Some of the antibodies used in these assays and the epitopes recognized by them are listed in Table 7.1 below. Details of all of the assays can be found in Tables 7.2 (also shown below).

TABLE 7.1

| Capture antibody (clone) | Detection antibody (clone) |
|---|---|
| anti-BMP-15 antibody 21 (111/39) - | anti-BMP-15 antibody 4 (87/38) - binding |
| binding to epitopes contained in peptides | to epitopes contained in peptides having |
| having SEQ ID NOS: 8-11, 32, 45, and 46 | SEQ ID NOS: 8, 9, and 48-50 |
| anti-BMP-15 antibody 1A (114/1) | anti-BMP-15 antibody 4 (87/38) - binding |
| | to epitopes contained in peptides having |
| | SEQ ID NOS: 8, 9, and 48-50 |
| anti-BMP-15 antibody 14 (94/29) | anti-BMP-15 antibody 4 (87/38) - corresponding |
| | to peptide numbers 8, 9, and 48-50 |
| anti-BMP-15 antibody 7A (114/35) | anti-BMP-15 antibody 4 (87/38) - binding |
| | to epitopes contained in peptides having |
| | SEQ ID NOS: 8, 9, and 48-50 |
| anti-BMP-15 antibody 23A (128/98A) | anti-BMP-15 antibody 23A (128/98A) |
| anti-BMP-15 antibody 1A (114/1) | anti-BMP-15 antibody 6 (87/63) - binding |
| | to epitopes contained in peptides having |
| | SEQ ID NOS: 8, 10, 11, 28, 40, 49-50 |
| anti-BMP-15 antibody 7A (114/35) | anti-BMP-15 antibody 6 (87/63) - binding |
| | to epitopes contained in peptides having |
| | SEQ ID NOS: 8, 10, 11, 28, 40, 49-50 |
| anti-BMP-15 antibody 1A (114/1) | anti-BMP-15 antibody 6 (87/63) - binding |
| | to epitopes contained in peptides having |
| | SEQ ID NOS: 8, 10, 11, 28, 40, 49-50 |
| anti-BMP-15 antibody 21 (111/39) - | anti-BMP-15 antibody 4 (87/38) - binding |
| binding to epitopes contained in peptides | to epitopes contained in peptides having |
| having SEQ ID NOS: 8-11, 32, 45, and 46 | SEQ ID NOS: 8, 9, and 48-50 |
| anti-BMP-15 antibody 23A (128/98A) | anti-BMP-15 antibody 23A (128/98A) |

TABLE 7.2

| Plt 1 | 1 | 2 | 3 | 1 | 2 | 3 | 4 | 5 | 6 | 7 | 8 | 9 | 10 | 11 | 12 |
|---|---|---|---|---|---|---|---|---|---|---|---|---|---|---|---|
| A | Act A | complex | Bovine | 0.097 | 0.182 | 0.086 | 0.108 | 0.484 | 0. [symbol] 71 | 0.061 | 0.202 | 0.068 | 0.156 | 0.499 | 0.102 |
| | Matrix | 1:10000 | FF | | | | | | | | | | | | |
| | | | 11:2 | | | | | | | | | | | | |
| B | BMP-15 | Human | Equine | 0.738 | 0.091 | 0.074 | 0.826 | 0.079 | 0.0 [symbol] 5 | 0.86 | 0.07 | 0.063 | 0.757 | 0.101 | 0.081 |
| | (P + M) | FF | pool | | | | | | | | | | | | |
| | 0.18 n mL | 2 neat | neat | | | | | | | | | | | | |
| C | BMP-15 | Human | Equine | 1.593 | 0.09 | 0.082 | 1.747 | 0.076 | 0.5 [symbol] 3 | 1.288 | 0.07 | 0.062 | 1.612 | 0.1 | 0.095 |
| | (P + M) | FF | pool | | | | | | | | | | | | |
| | 0.5 n mL | 2 1:2 | 1:2 | | | | | | | | | | | | |
| D | BMP-15 | Human | Canine | 3.709 | 0.092 | 0.198 | 3.761 | 0.093 | 0.143 | 3.248 | 0.071 | 0.077 | 3.667 | 0.104 | 0.166 |
| | (P + M) | FF | ext | | | | | | | | | | | | |
| | 1.6 ng/mL | 3 neat | 1:10 | | | | | | | | | | | | |
| E | BMP-15 | Human | Canine | 4.186) | 0.096 | 0.189 | 4.257 | 0.078 | 0.164 | 4. [symbol] 52 | 0.052 | 0.079 | 4.208 | 0.104 | 0.189 |
| | (P + M) | FF | ext | | | | | | | | | | | | |
| | 5 ng/mL | 3 1:2 | 1:20 | | | | | | | | | | | | |
| F | BMP-15 | Human | Mouse | 0.139 | 0.0 [symbol] 2 | 1.028 | 0.22 | 0.064 | 0.211 | 0.092 | 0.0 [symbol] 9 | 0.075 | 0.228 | 0.075 | 0.181 |
| | (Mature) | FF | Testis ext | | | | | | | | | | | | |
| | 1 ng/mL | A4 neat | 1:10 | | | | | | | | | | | | |
| G | GDF-9 | Human | Mouse | 0.127 | 0.102 | 0.894 | 0.12 | 0.082 | 0.165 | 0.071 | 0.073 | 0.088 | 0.169 | 0.099 | 0.189 |
| | (P + M) | FF | Testis ext | | | | | | | | | | | | |
| | (5 ng/mL) | A41:2 | 1:20 | | | | | | | | | | | | |
| H | mouse | Bovine | | 0.099 | 0.095 | | 0.107 | 0.063 | | 0.067 | 0.08 | | 0.18 | 0.084 | |
| | EMP-15 | FF | | | | | | | | | | | | | |
| | 1:10000 | 1 neat | | | | | | | | | | | | | |

| | | | | |
|---|---|---|---|---|
| Capture | 111/39 (#21) | 114/1 (#1A) | 94/29 (#14) | 114/35 (#7A) |
| Detection | 87/38 (#4) | 87/38 (#4) | 87/38 (#4) | 87/38 (#4) |

| | 1 | 2 | 3 | 1 | 2 | 3 | 4 | 5 | 6 | 7 | 8 | 9 |
|---|---|---|---|---|---|---|---|---|---|---|---|---|
| P [symbol] t 2 | | | | | | | | | | | | |
| A | Act A | complex | Bovine FF | 0.05 [symbol] | 0.115 | 0.052 | 0.08 | 0.224 | 0.052 | 0.0 [symbol] 9 | 0.191 | 0.038 |
| | Matrix | 1:10000 | 11:2 | | | | | | | | | |
| B | BMP-15 | Human | Equine | 0.14 | 0.043 | 0.056 | 0.426 | 0.045 | 0.055 | 0.536 | 0.041 | 0.053 |
| | (P + M) | FF | pool | | | | | | | | | |
| | 0. [symbol] 8 ng/mL | 2 neat | neat | | | | | | | | | |
| C | BMP-15 | Human | Equine | 0.2 [symbol] | 0.04 | 0.046 | 0.934 | 0.048 | 0.085 | 0.823 | 0.04 | 0.0 [symbol] 2 |
| | (P + M) | FF | pool | | | | | | | | | |
| | 0.5 ng/mL | 2 1:2 | 1:2 | | | | | | | | | |
| D | BMP-15 | Human | Canine | 0.628 | 0.04 [symbol] | 0.0 [symbol] 1 | 2.382 | 0.0 [symbol] 1 | 0.131 | 2.142 | 0.039 | 0.1 |
| | (P + M) | FF | ext | | | | | | | | | |
| | 1. [symbol] ng/mL | 3 neat | 1: [symbol] 0 | | | | | | | | | |
| E | BMP-15 | Human | Canine | 1.71 | 0.067 | 0.054 | 4.16 | 0.049 | 0.132 | 4.08 | 0.04 | 0.1 |
| | (P + M) | FF | ext | | | | | | | | | |
| | 5 ng/mL | 3 1:2 | 1:20 | | | | | | | | | |
| F | BMP-15 | Human | Mouse | 0.986 | 0.04 | 0.0 [symbol] 4 | 0.113 | 0.044 | 0.11 | 0.112 | 0.032 | 0.074 |
| | (Mature) | FF | Testis | | | | | | | | | |
| | 1 ng/mL | A4 neat | 1:10 | | | | | | | | | |
| G | GDF-9 | Human | Mouse | 0.05 [symbol] | 0.0 [symbol] 9 | 0.054 | 0.082 | 0.04 [symbol] | 0.092 | 0.077 | 0.049 | 0.09 |
| | (P + M) | FF | Testis | | | | | | | | | |
| | (5 ng/mL) | A4 1:2 | ext 1:20 | | | | | | | | | |
| H | mouse | Bovine | | 0.0 [symbol] 8 | 0.043 | | 0.082 | 0.0 [symbol] 6 | | 0.0 [symbol] 9 | 0.039 | |
| | BMP-15 | FF | | | | | | | | | | |
| | 1:10000 | 1 neat | | | | | | | | | | |
| | | | Capture | 128/98 (#23A) | | | 114/1 (#1A) | | | 114/38 (#7A) | | |
| | | | Detection | 128/98 (#23A) | | | 87/63 (#6) | | | 87/63 (#6) | | |
| Plt 1 | | | | | | | | | | | | |
| A | Act A | BMP-15 | HETERO | 0.023 | 0.194 | 0.178 | 0.061 | 0.16 [symbol] | 0.152 | 0.054 | 4.16 | 0.137 |
| | Matrix | untag mature | Complex | | | | | | | | | |
| | | 2.5 ng/mL | 5 ng/mL | | | | | | | | | |
| B | BMP-15 | BMP-15 | HETERO | 0.05 | 0.329 | 0.1 | 0.092 | 0.254 | 0.105 | 0.06 | 4.173 | 0.094 |
| | (P + M) | mutant | Complex | | | | | | | | | |
| | 0.021 ng/mL | 5 ng/mL | 2.5 ng/mL | | | | | | | | | |
| C | BMP-15 | BMP-15 | GDF-9 | 0.1 | 0.173 | 0.03 | 0.141 | 0.141 | 0.06 | 0.086 | 4.252 | 0.0 [symbol] |
| | (P + M) | mutant | p + m | | | | | | | | | |
| | 0.062 ng/mL | 2.5 ng/mL | 5 ng/mL | | | | | | | | | |
| D | BMP-15 | Complex | BMP-15 | 0.233 | 0.98 | 4.159 | 0.27 | 0.803 | 4.201 | 0.167 | 0.669 | 2.973 |
| | (P + M) | 1:2000 | p + m | | | | | | | | | |
| | 0.185 ng/mL | | lyo 5 ng/mL | | | | | | | | | |
| E | BMP-15 | Complex | BMP-15 | 0.658 | 0.325 | 3.233 | 0.674 | 0.245 | 0.355 [symbol] | 0.419 | 0.263 | 1.695 |
| | (P + M) | 1:6000 | p + m | | | | | | | | | |
| | 0.56 ng/mL | | lyo 2.5 ng/mL | | | | | | | | | |
| F | BMP-15 | Marty's | BMP-15 | 1.788 | 4.311 | 1.803 | 1.867 | 4.361 | 2.02 [symbol] | 1.143 | 4.378 | 0.962 |
| | (P + M) | BMP-15 | p + m | | | | | | | | | |
| | 1.67 ng/mL | 5 ng/mL | lyo 1.25 ng/mL | | | | | | | | | |
| G | BMP-15 | Marty's | BMP-15 m | 3. [symbol] 77 | 3.901 | 0.221 | 4.128 | 4.205 | 0.186 | 3.193 | 4.19 | 4.262 |
| | (P + M) | BMP-15 | lyo 5 ng/mL | | | | | | | | | |
| | 5 ng/mL | 0.5 ng/mL | | | | | | | | | | |
| H | BMP-15 untag | Marty's | BMP-15 m | 0.347 | 0.53 | 0.121 | 0.311 | 0.934 | 0.131 | 4.395 | 0.884 | 4.34 [symbol] |
| | mature | BMP-15 | lyo 2.5 ng/mL | | | | | | | | | |
| | 5 ng/mL | 0.0 [symbol] ng/mL | | | | | | | | | | |

| | | | |
|---|---|---|---|
| Capture | 11411 (#1A) | 111139 (#21A) | 128/98 (#23A) |
| Detection | 87/63 (#6) | 87/38 (#4) | 128/98 (#23A) |
| Sample Size | 50 μl | 50 μl | 100 μl |

| | |
|---|---|
| Procedure | sample* + 50 uL ASB-114 ---> 2 hr inc. @ RT ---> 100 uL Biotins 5K in CND-123 -- [symbol] > |
| | 60 min @ RT ---> 100 uL SHRP 27K ---> 30 min @ RT--->100 uL TMB ---> 10 min @RT |

[symbol]

indicates data missing or illegible when filed

### Example 8: Development of Single Epitope BMP-15 ELISA (Mature-Mature) with Sensitivity of 1 pg/mL

The antibody used for this ELISA was an anti-BMP-15 monoclonal antibody 23A (clone #128/98A) that recognizes an epitope in the mature region of the BMP-15.

### BMP-15, 23A/23A (Mat-Mat)

| Well ID | O.D (Rep.) | O.D. (Mean) | % CV |
|---|---|---|---|
| BLK | 0.048 | 0.048 | n/a |

Using the above ELISA, full-length recombinant BMP-15 including both the pro and mature regions (pro+mature) was detected at various concentrations (see Table 8 below). A linear relationship between concentration of the full-length BMP-15 and the detection signal was obtained (FIG. 9).

TABLE 8

| | O.D. | O.D. | | Conc. | Conc. | Pred. Conc. |
|---|---|---|---|---|---|---|
| Well 10 | (Rep.) | (Mean) | % CV | (pg/ml) | (Rep.) | (Mean) |
| STD1 | 0.081 | 0.079 | 3.580 | 2.75 | 3.454 | 3.352 |
| | 0.077 | | | 2.75 | 3.250 | |
| STD2 | 0.112 | 0.114 | 2.481 | 5.5 | 5.101 | 5.211 |
| | 0.116 | | | 5.5 | 5.321 | |
| STD3 | 0.189 | 0.187 | 1.513 | 11 | 9.575 | 9.453 |
| | 0.185 | | | 11 | 9.331 | |
| STD4 | 0.350 | 0.343 | 2.676 | 23 | 20.098 | 19.651 |
| | 0.337 | | | 23 | 19.203 | |
| STD5 | 0.676 | 0.683 | 1.449 | 44 | 44.380 | 44.934 |
| | 0.690 | | | 44 | 45.488 | |
| STD6 | 1.288 | 1.285 | 0.385 | 89 | 96.406 | 96.091 |
| | 1.281 | | | 89 | 95.776 | |
| STD7 | 2.235 | 2.228 | 0.412 | 169 | 187.135 | 186.480 |
| | 2.222 | | | 169 | 185.825 | |
| STD8 | 3.544 | 3.543 | 0.040 | 336 | 325.909 | 325.798 |
| | 3.542 | | | 336 | 325.687 | |

### Example 9: Development of BMP-15 ELISA (Pro-Pro)

The antibodies used for this ELISA were anti-BMP-15 monoclonal antibodies 21 (clone #111-39A) and 4 (clone #87/38A). Antibody 21 specifically recognizes epitopes having SEQ ID NOS: 8-11, 32, 45, and 46; and antibody 4 specifically recognizes epitopes SEQ ID NOS: 8, 9, 48, 49, and 50, on BMP-15.

### BMP-15, 21/4 (Pro-Pro)

| Well ID | O.D (Rep.) | O.D. (Mean) | % CV |
|---|---|---|---|
| BLK | 0.070 | 0.070 | n/a |

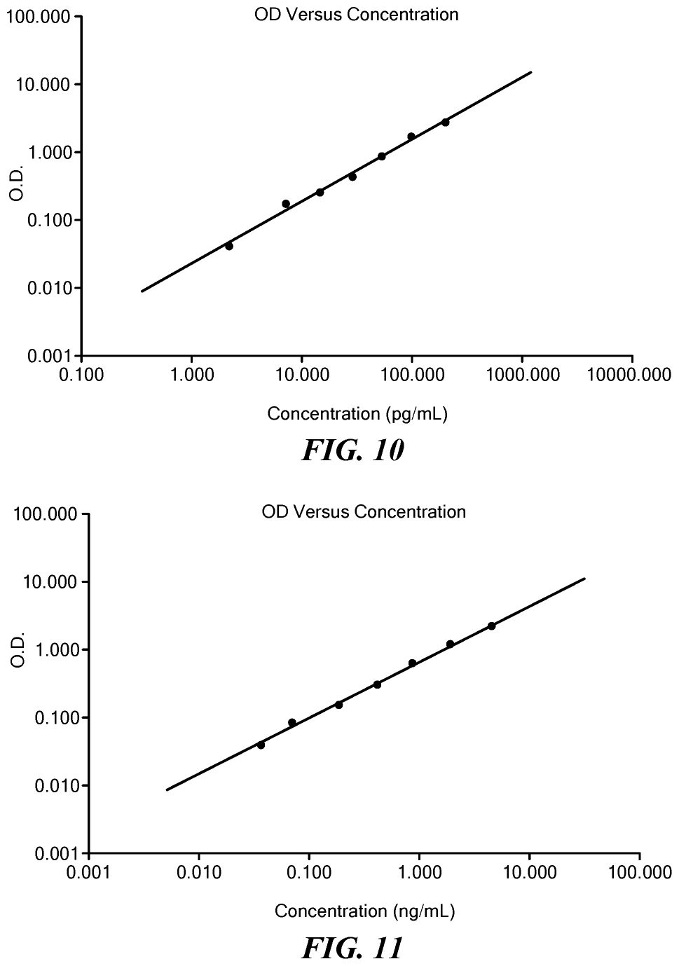

Using the above ELISA, full-length recombinant BMP-15 (including both the pro and mature regions, i.e., pro+mature) was detected at various concentrations (see Table 7 below). A linear relationship between concentration of full-length BMP-15 and the detection signal was obtained (FIG. 10).

### BMP-15 P+M, U0114, Lyo. 100516

TABLE 7

| | O.D. | O.D. | | Conc. | Conc. | Pred. Conc. |
|---|---|---|---|---|---|---|
| Well ID | (Rep.) | (Mean) | % CV | (pg/ml) | (Rep.) | (Mean) |
| STD1 | 0.044 | 0.044 | n/a | 2.2 | 2.020 | 2.020 |
| STD2 | 0.175 | 0.175 | n/a | 7.5 | 9.119 | 9.119 |
| STD3 | 0.265 | 0.255 | n/a | 15 | 14.344 | 14.344 |
| STD4 | 0.467 | 0.467 | n/a | 29 | 26.627 | 26.627 |
| STD5 | 0.887 | 0.887 | n/a | 54 | 53.640 | 53.640 |
| STD6 | 1.686 | 1.686 | n/a | 103 | 108.146 | 108.146 |
| STD7 | 2.955 | 2.955 | n/a | 204 | 199.556 | 199.556 |

### Example 10: Development of GDF-9 ELISA (Mature-Mature)

The antibodies used for this ELISA were anti-GDF-9 monoclonal antibody 17A (clone #114-58A) which recognizes epitopes having SEQ ID NOS: 123, 135, and 136; and anti-GDF-9 monoclonal antibody 6 (clone #98-74A) which recognizes epitopes having SEQ ID NOS: 123, 129, 132, 144, 145, and 146.

### GDF-9, 17A/6

| Well ID | O.D. | O.D. (Mean) | % CV |
|---|---|---|---|
| BLK | 0.065 | 0.066 | 2.143 |
| | 0.067 | | |

Using the above ELISA, recombinant GDF-9 was detected at various concentrations (see Table 8 below). A linear relationship between concentration of full-length recombinant GDF-9 (pro+mature) and signals detected using this ELISA and the detection signal was obtained (FIG. 11).

| | O.D. | O.D. | | Conc. | Pred. | Pred. Conc. |
|---|---|---|---|---|---|---|
| Well ID | (Rep.) | (Mean) | % CV | (ng/mL) | Conc. | (Mean) |
| STD1 | 0.043 | 0.042 | 1.664 | 0.036 | 0.035 | 0.034 |
| | 0.042 | | | 0.036 | 0.034 | |
| STD2 | 0.085 | 0.083 | 2.541 | 0.071 | 0.078 | 0.077 |
| | 0.082 | | | 0.071 | 0.075 | |
| STD3 | 0.170 | 0.170 | 0.000 | 0.182 | 0.178 | 0.178 |
| | 0.170 | | | 0.182 | 0.178 | |
| STD4 | 0.330 | 0.329 | 0.430 | 0.409 | 0.390 | 0.389 |
| | 0.328 | | | 0.409 | 0.387 | |
| STD5 | 0.649 | 0.648 | 0.218 | 0.866 | 0.873 | 0.871 |
| | 0.647 | | | 0.866 | 0.869 | |
| STD6 | 1.280 | 1.290 | 1.096 | 1.892 | 1.971 | 1.990 |
| | 1.300 | | | 1.892 | 2.008 | |
| STD7 | 2.516 | 2.469 | 2.692 | 4.483 | 4.485 | 4.383 |
| | 2.422 | | | 4.483 | 4.280 | |

### Example 9: Development of GDF-9 ELISA for Animal-Derived Sample (Mature-Mature)

Several antibody pairs were used in this set of immunoassays. The design of the assays is evident from Table 9 below. For one group of four assays, a different anti-GDF-9 antibody was used for capture in each assay. These antibodies are: 17A (clone 114/58A) recognizing epitopes having SEQ ID NOS: 123, 135, and 136; 13A (clone 114/29A) recognizing epitopes having SEQ ID NOS: 111, 123, and 132; 14A (clone 114/31), and 12A (clone 114/5). Each of these four assays used one anti-GDF-9 antibody, 6 (clone 98/74) for detection. This antibody is specific for epitopes having SEQ ID NOS: 123, 129, 132, 144, 145, and 146.

In a second group of four assays, the anti-GDF-9 antibody pairs used were as follows:

(1) antibody 6 (clone 98/74) binding to epitopes contained in peptides having SEQ ID NOS: 123, 129, 132, 144, 145, and 146, for capture, and antibody 14A (clone 114/31) for detection;

(2) antibody 25 (clone 114/50) for capture, and antibody 6 (clone 98/74) binding to epitopes contained in peptides having SEQ ID NOS: 123, 129, 132, 144, 145, and 146, for detection;

(3) antibody 16A (clone 114/31) for capture and antibody 6 (clone 98/74) binding to epitopes contained in peptides having SEQ ID NOS: 123, 129, 132, 144, 145, and 146, for detection; and

(4) antibody 6 (clone 98/74) binding to epitopes contained in peptides having SEQ ID NOS: 123, 129, 132, 144, 145, and 146, for capture, and antibody 16A (clone 114/50A) for detection.

TABLE 9

| | 1 | 2 | 3 | 1 | 2 | 3 | 4 | 5 | 6 | 7 | 8 | 9 | 10 | 11 | 12 |
|---|---|---|---|---|---|---|---|---|---|---|---|---|---|---|---|
| Plt 1 | | | | | | | | | | | | | | | |
| A | Act A | [symbol] ovine | Bovine | 0.028 | 0.549 | 0.554 | 0.03 [symbol] | 0.035 | 0.053 | 0.035 | 0.424 | 0.055 | 0.0 [symbol] | 0.534 | 0.059 |
| | Matrix | GDF- [symbol] | FF | | | | | | | | | | | | |
| | | 1:10000 | 1 neat | | | | | | | | | | | | |
| B | GDF-9 | complex | Bovine | 0.213 | 1.486 | 0.041 | 0.227 | 1.218 | [symbol] .042 | 0.18 [symbol] | 1.207 | 5.05 | 0.192 | 1.206 | 0.052 |
| | (P + M) | 1:10000 | FF | | | | | | | | | | | | |
| | 0.18 ng/mL | | 1 1:2 | | | | | | | | | | | | |
| C | GDF-9 | Human | Equine | 0.501 | 0.0 [symbol] | 0.027 | 0. [symbol] 47 | [symbol] .0 [symbol] | 0.03 [symbol] | 0.43 [symbol] | 0.063 | 0.036 | 0.414 | 0.0 [symbol] | 0.045 |
| | (P + M) | FF | pool | | | | | | | | | | | | |
| | 0.5 ng/mL | 2 neat | neat | | | | | | | | | | | | |
| D | GDF-9 | Human | Equine | 1.536 | 0.042 | 0.027 | [symbol] 29 | 0.03 [symbol] | 0.031 | 1.3 [symbol] | 0.047 | 0.034 | 1.213 | 0.045 | 0.044 |
| | (P + M) | FF | pool | | | | | | | | | | | | |
| | 1. [symbol] ng/mL | 2 1:2 | 1:2 | | | | | | | | | | | | |
| E | GDF-9 | Human | Canine | | 0.04 | 0.039 | | 0.03 [symbol] | 0.04 [symbol] | | 0.068 | 0.058 | 3.19 [symbol] | 0.047 | 0.082 |
| | (P + M) | FF | ext | | | | | | | | | | | | |
| | 5 ng/mL | 3 neat | 1:10 | | | | | | | | | | | | |
| F | GDF-9 | Human | Canine | 0.818 | 0.03 [symbol] | 0.034 | 0.7 [symbol] | 0.03 [symbol] | 0.041 | 0.882 | 0.04 | 0.04 [symbol] | 0. [symbol] 7 | 0.037 | 0.064 |
| | (Mature) | FF | ext | | | | | | | | | | | | |
| | [symbol] ng/mL | 3 1:2 | 1:20 | | | | | | | | | | | | |
| G | BMP-15 | Human | Mouse | 0.026 | 0.047 | 0.035 | 0.031 | 0.047 | 0.044 | 0.031 | 0. [symbol] 52 | 0.045 | 0.046 | 0.051 | 0.062 |
| | (P + M) | FF | Testis ext | | | | | | | | | | | | |
| | (5 ng/mL) | A4 neat | 1:10 | | | | | | | | | | | | |
| H | mouse | Human | Mouse | 0.395 | 0.039 | 0.034 | 0.378 | 0.038 | 0.04 | 0.349 | 0.047 | 0.0 [symbol] | 0.355 | 0.049 | 0.0 [symbol] |
| | GDF-9 | FF | Testis ext | | | | | | | | | | | | |
| | 1:10000 | A41:2 | 1:20 | | | | | | | | | | | | |
| | | | Capture | 114/58 (#17A) | | | 114/29 (#13A) | | | 114/31 (#14A) | | | 114/5 (#12A) | | |
| | | | Detection | 88/74 (#6) | | | 88/74 (#6) | | | 88/74 (#6) | | | 88/74 (#6) | | |
| | | | | TOTAL HUMAN | | | | | | | Total hGDF | | | EQUINE | |
| | | | | (M − M) | | | | | | | (M − M) | | | (M − M) | |
| Plt 2 | | | | | | | | | | | | | | | |
| A | Act A | [symbol] ovine GDF-9 | Bovine | 0.0 [symbol] 1 | 0.50 [symbol] | 0.032 | 0.027 | 0.067 | 0.033 | 0.025 | 0.0 [symbol] | 0.025 | 0.042 | 1.512 | 0.037 |
| | Matrix | 1:10000 | FF | | | | | | | | | | | | |
| | | | 1 neat | | | | | | | | | | | | |
| B | GDF-9 | complex | Bovine | 0.114 | 0.35 [symbol] | 0.029 | 0.118 | 0.106 | 0.028 | 0.1 | 0.3 [symbol] 7 | 0.023 | 0.0 [symbol] | 0.5 [symbol] | 0.031 |
| | (P + M) | 1:10000 | FF | | | | | | | | | | | | |
| | 0.18 ng/mL | | 1 1:2 | | | | | | | | | | | | |
| C | GDF-9 | Human | Equine | 0.241 | 0.029 | 0.029 | 0.236 | 0.027 | 0.029 | 0.21 [symbol] | 0.02 [symbol] | 0.025 | 0.194 | 0.038 | 0.037 |
| | (P + M) | FF | pool | | | | | | | | | | | | |
| | 0. [symbol] ng/mL | 2 neat | neat | | | | | | | | | | | | |
| D | GDF-9 | Human | Equine | 0.73 [symbol] | 0.02 [symbol] | 0.031 | 0.7 [symbol] 2 | 0.03 | 0.027 | 0.617 | 0.023 | 0.021 | 0.564 | 0.03 | 0.031 |
| | (P + M) | FF | pool | | | | | | | | | | | | |
| | [symbol] 6 ng/mL | 2 1:2 | 1:2 | | | | | | | | | | | | |
| E | GDF-9 | Human | Canine | 2.1 [symbol] 8 | 0.023 | 0.053 | 2.1 [symbol] | 0.025 | 0.06 [symbol] | 1.807 | 0.024 | 0.0 [symbol] 4 | 1.729 | 0.033 | 0.0 [symbol] 4 |
| | (P + M) | FF | ext | | | | | | | | | | | | |
| | 5 ng/mL | 3 neat | 1:10 | | | | | | | | | | | | |
| F | GDF-9 | Human | Canine | 0.303 | 0.029 | 0.045 | 0.028 | 0.025 | 0.046 | 0.077 | 0.02 | 0.036 | 0.292 | 0.02 [symbol] | 0.049 |
| | (Mature) | FF | ext | | | | | | | | | | | | |
| | 1 ng/mL | 3 1:2 | 1:20 | | | | | | | | | | | | |
| G | BMP-15 | Human | Mouse | 0.029 | 0.022 | 0.074 | 0.025 | 0.028 | 0.05 [symbol] | 0.002 | 0.023 | 0.032 | 0.033 | 0.032 | 0.084 |
| | (P + M) | FF | Testis ext | | | | | | | | | | | | |
| | (5 ng/mL) | A4 neat | 1:10 | | | | | | | | | | | | |
| H | mouse | Human | Mouse | 0.739 | 0.026 | 0.067 | 0.02 [symbol] | 0.0 [symbol] 6 | 0.0 [symbol] | 0.007 | 0.02 [symbol] | 0.041 | 0.2 [symbol] 1 | 0.03 [symbol] | 0.075 |
| | GDF-8 | FF | Testis ext | | | | | | | | | | | | |
| | 1:10000 | A4 1:2 | 1:20 | | | | | | | | | | | | |

| | | | | |
|---|---|---|---|---|
| Capture | 88/74 (#6) | 99/ [symbol] 9 (#25) | 114/50 (#1 [symbol] A) | 98/74 (#6) |
| Detection | 114/31(#14A) | 9 [symbol] /74 (#6) | 9 [symbol] /74 (#6) | 114/16 (#4A) |

| | | |
|---|---|---|
| Mouse | GDF SPECIFIC Canine | OVINE |
| (M − M) | ( [symbol] − M) | (M − M) |

[symbol]

indicates data missing or illegible when filed

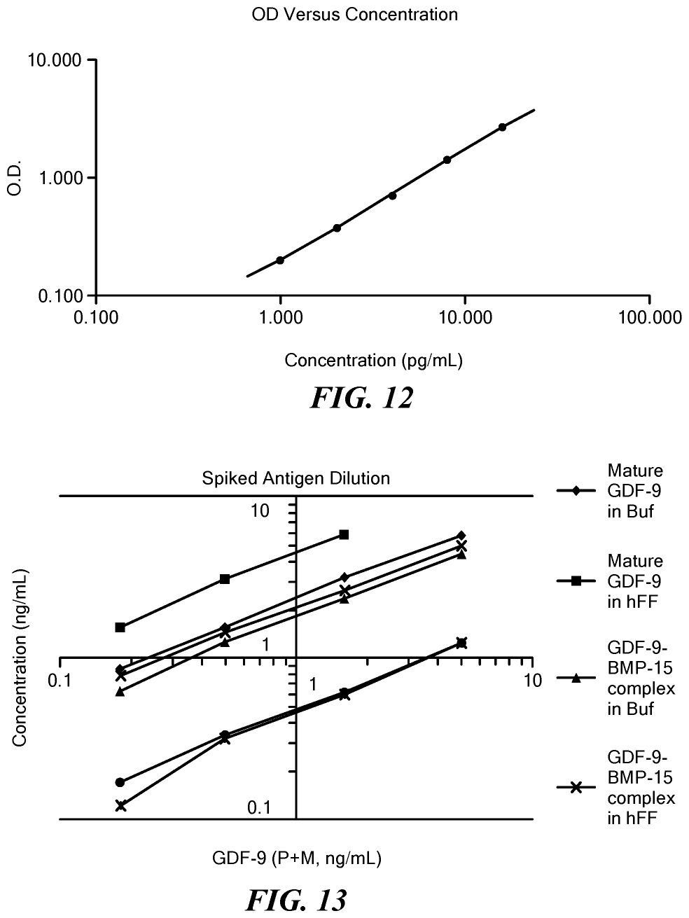

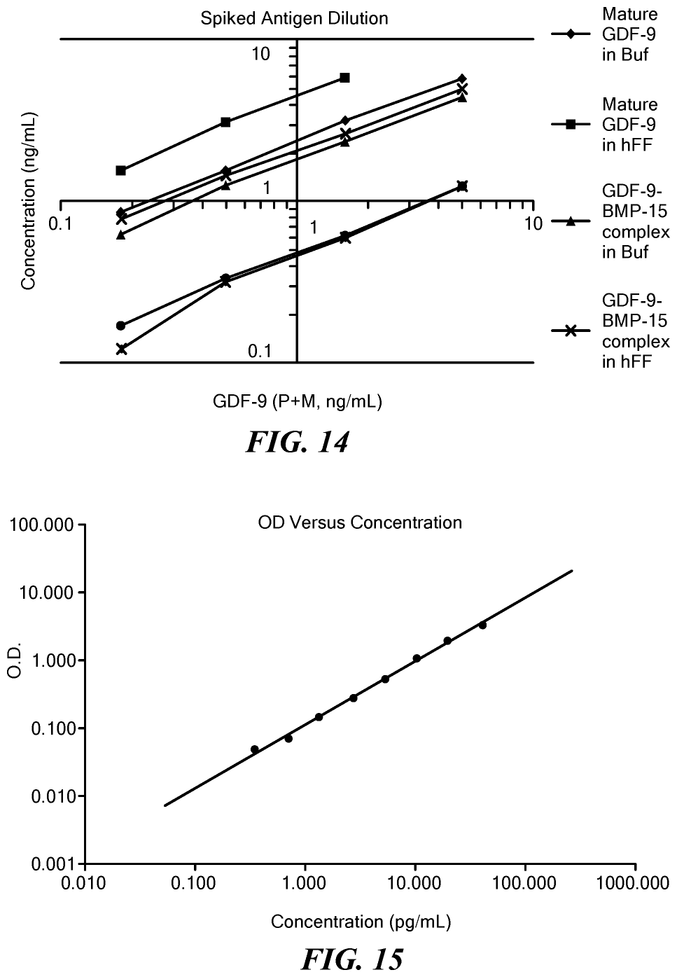

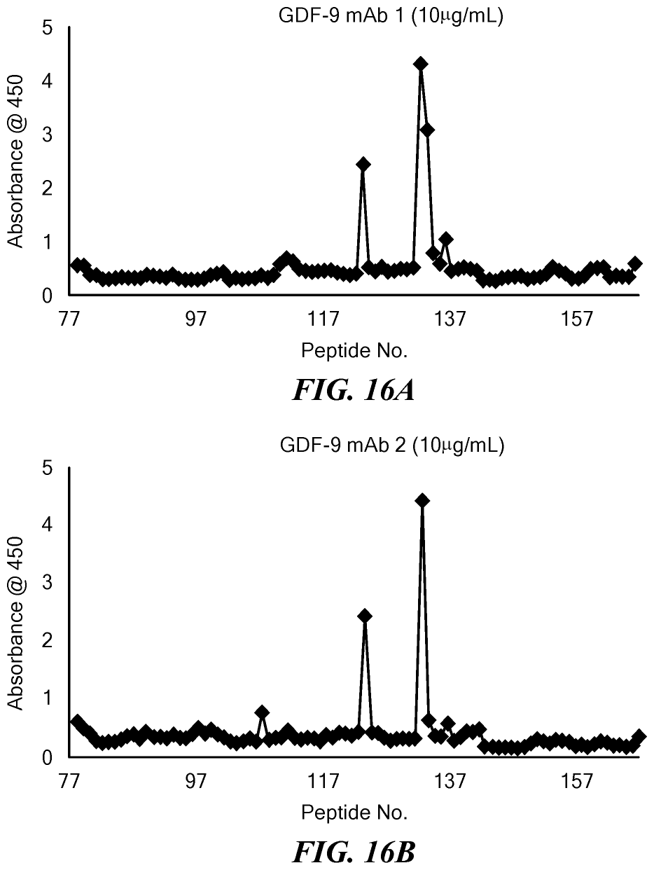

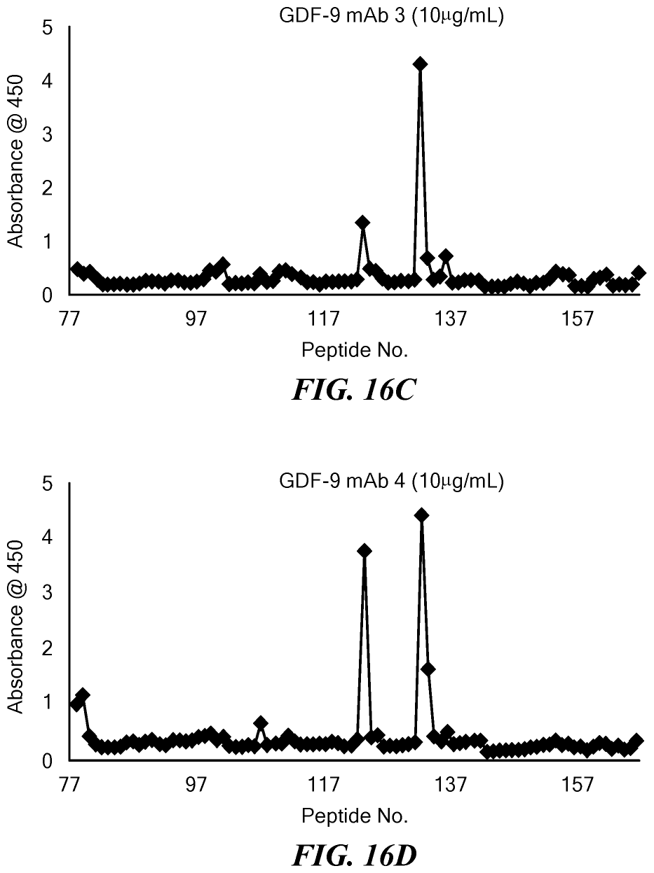

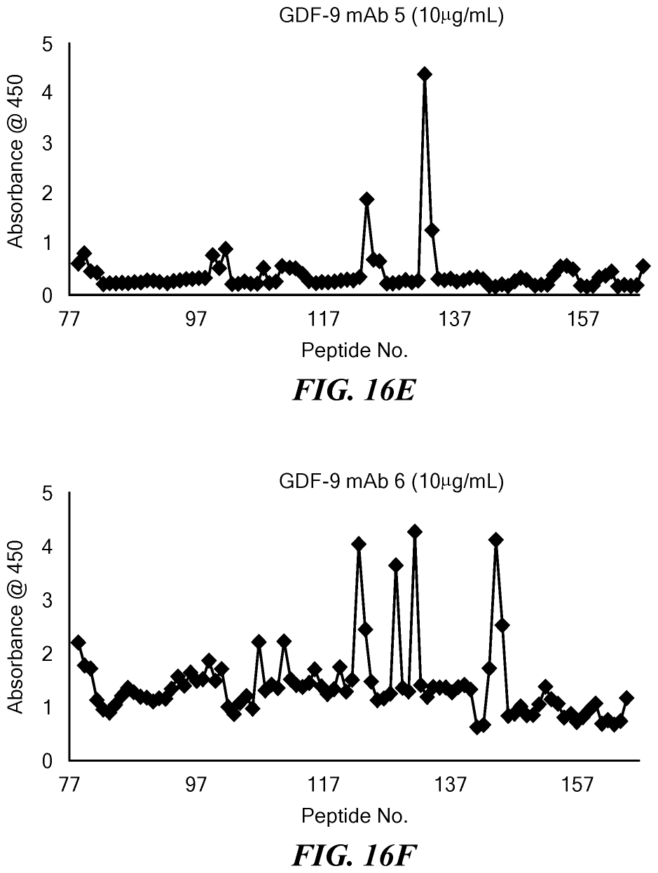

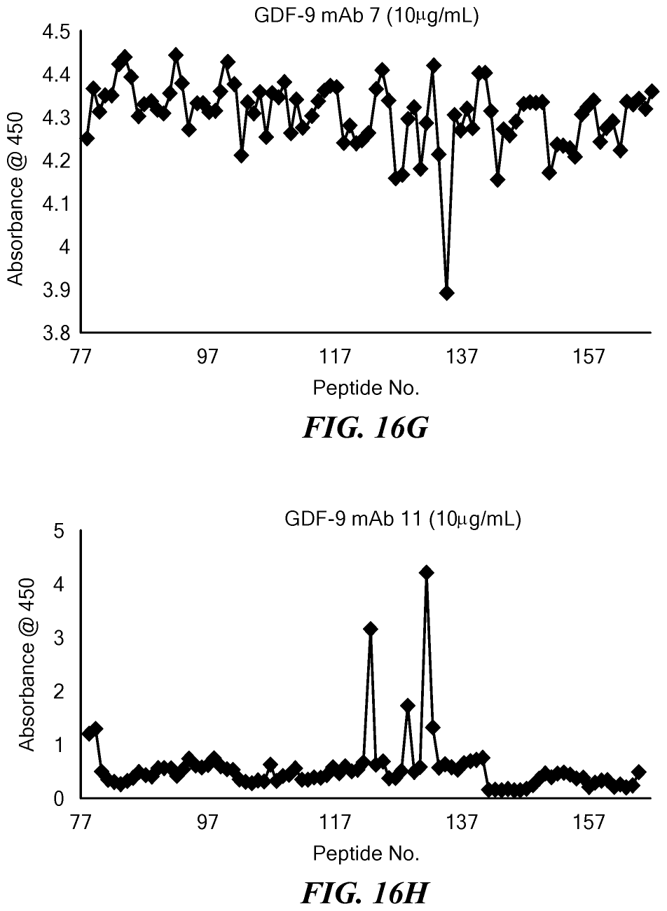

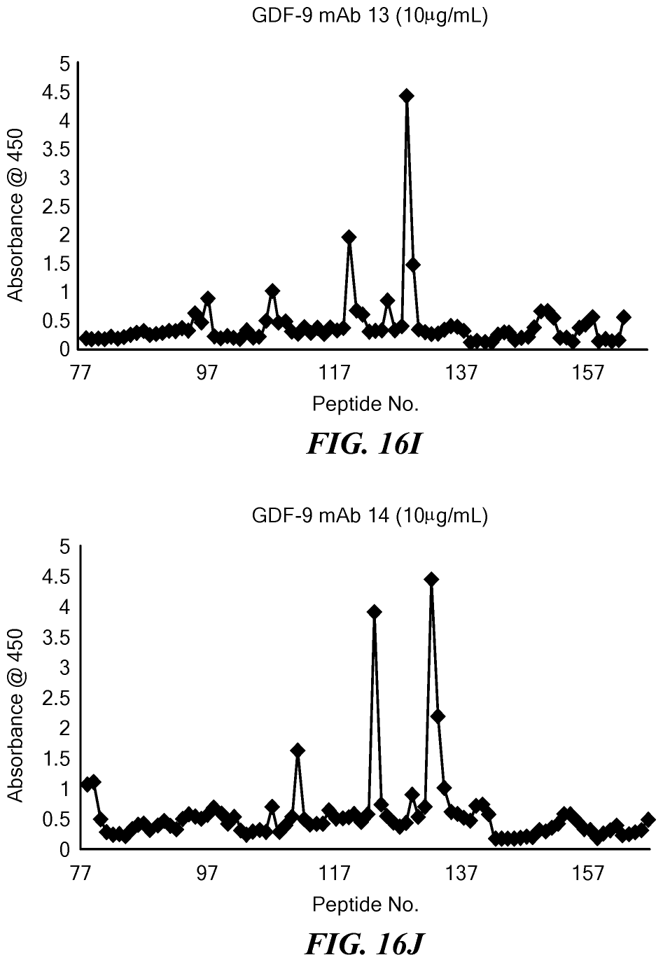

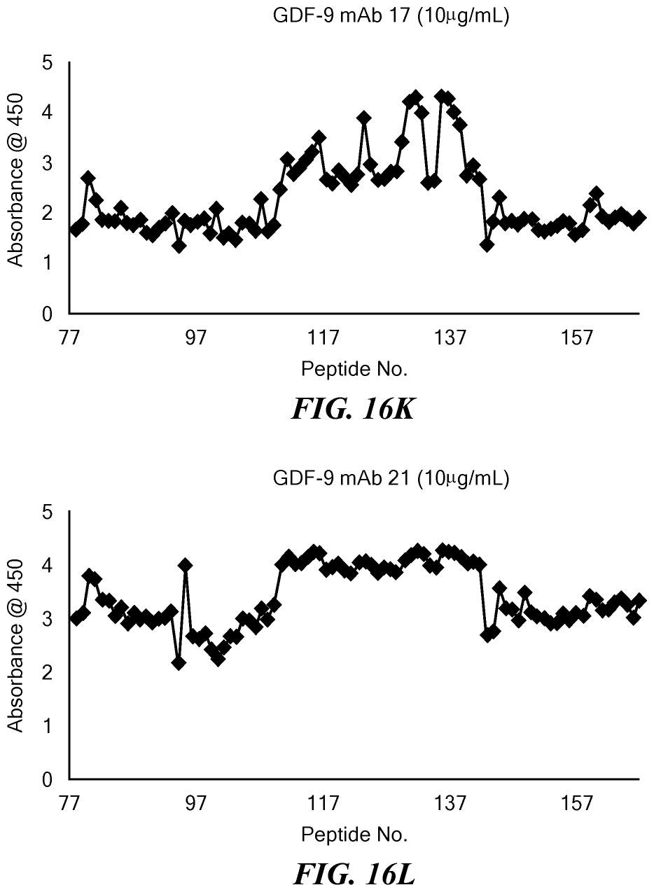

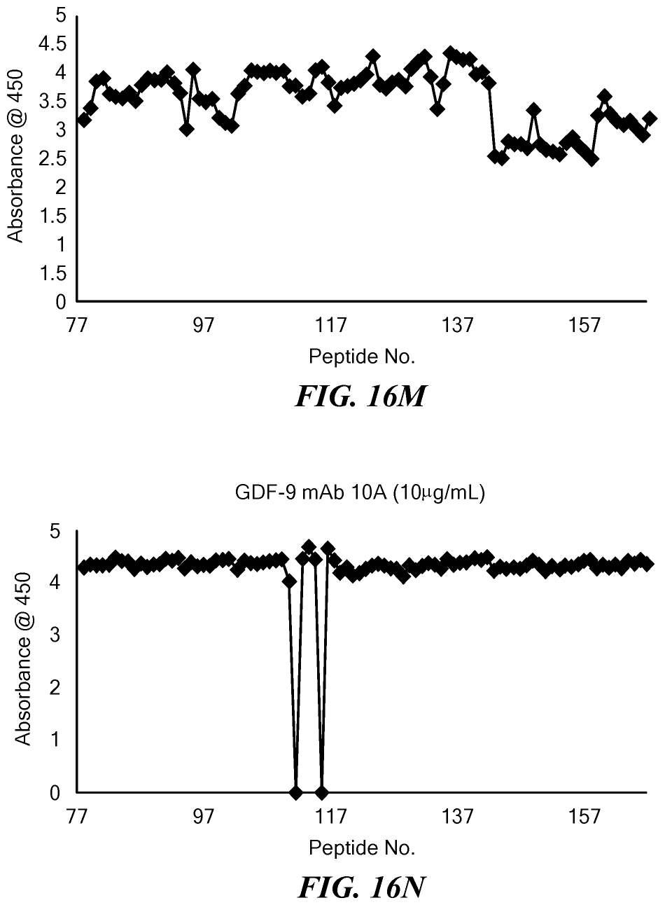

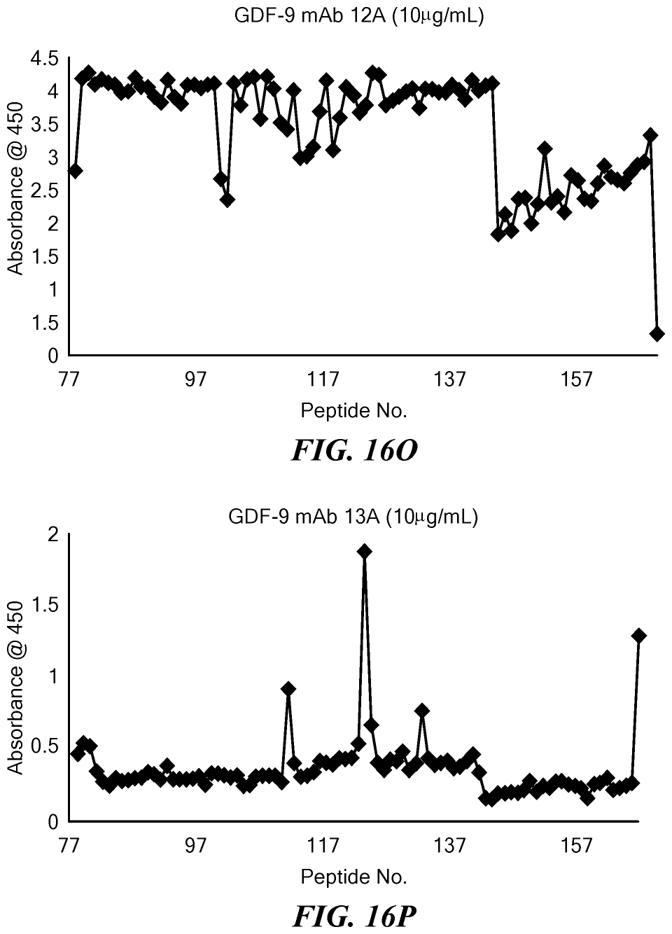

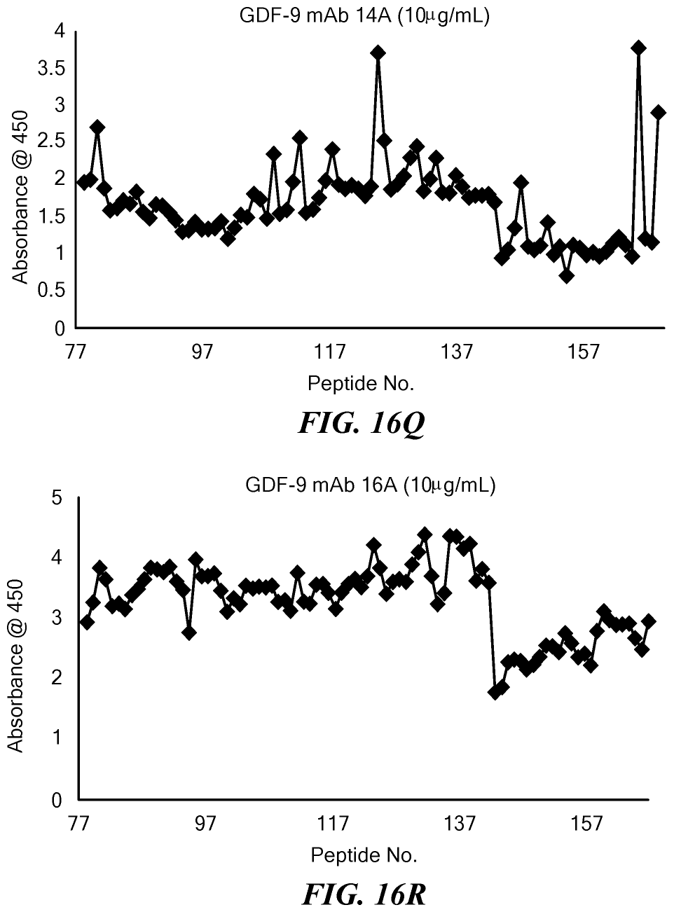

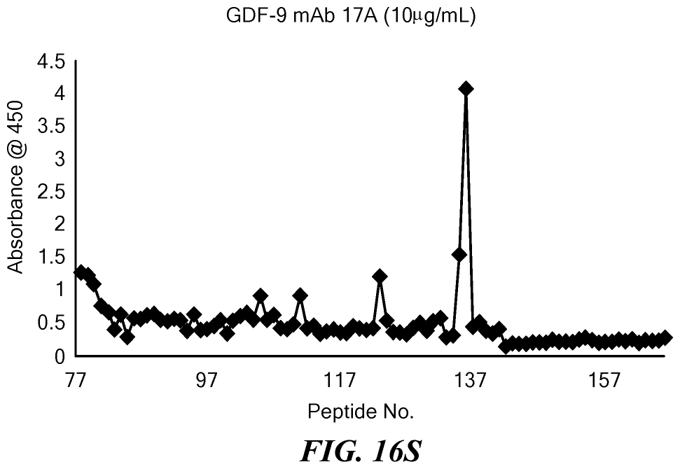

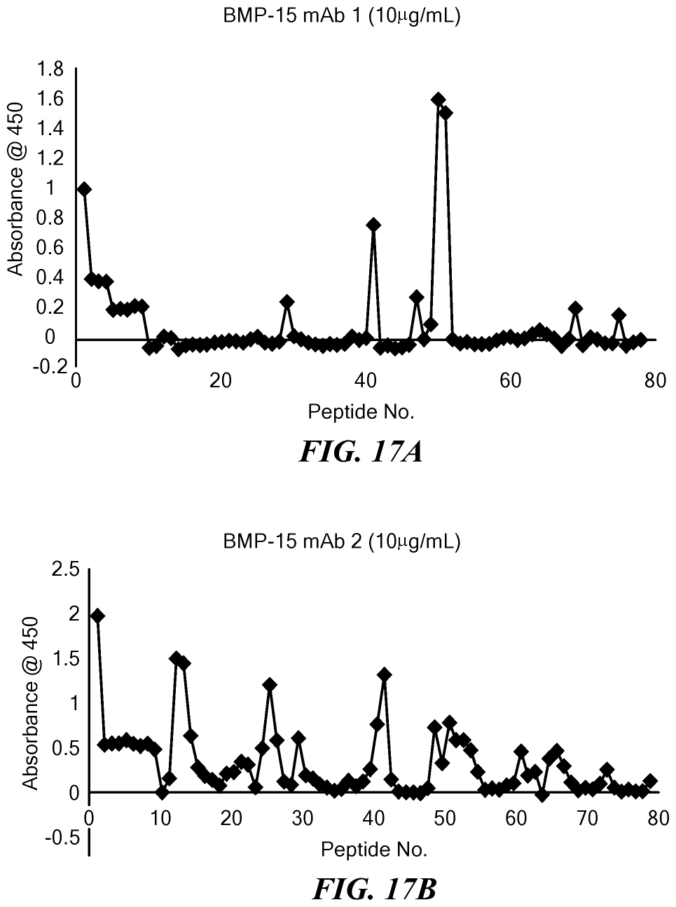

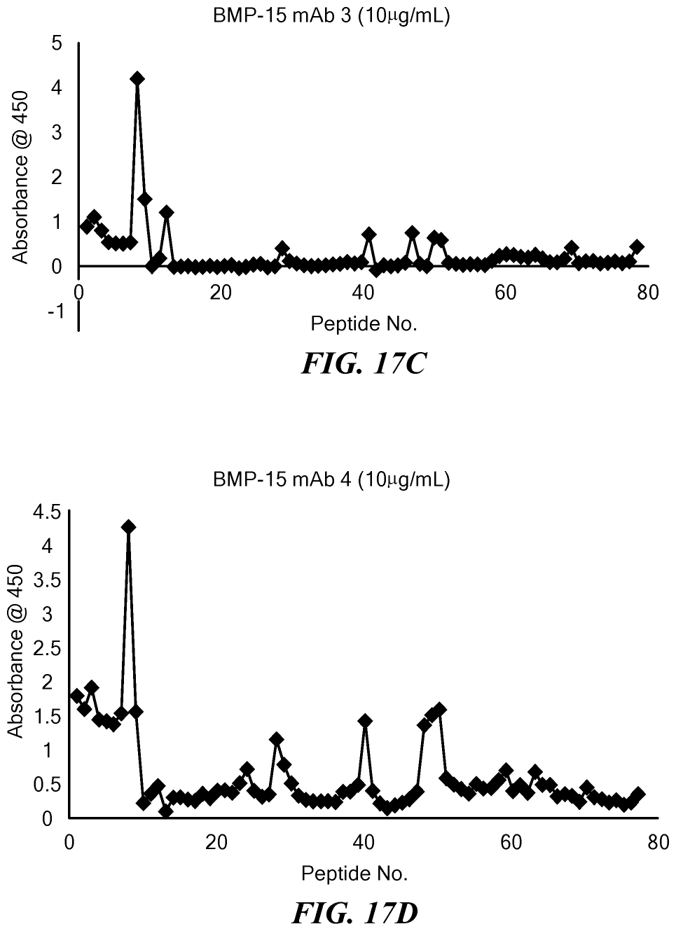

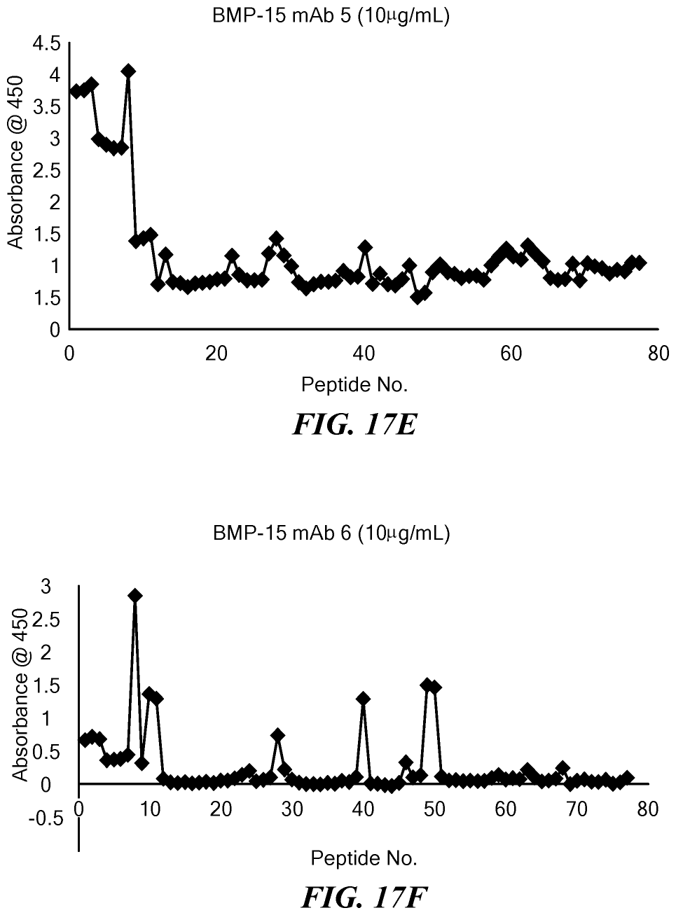

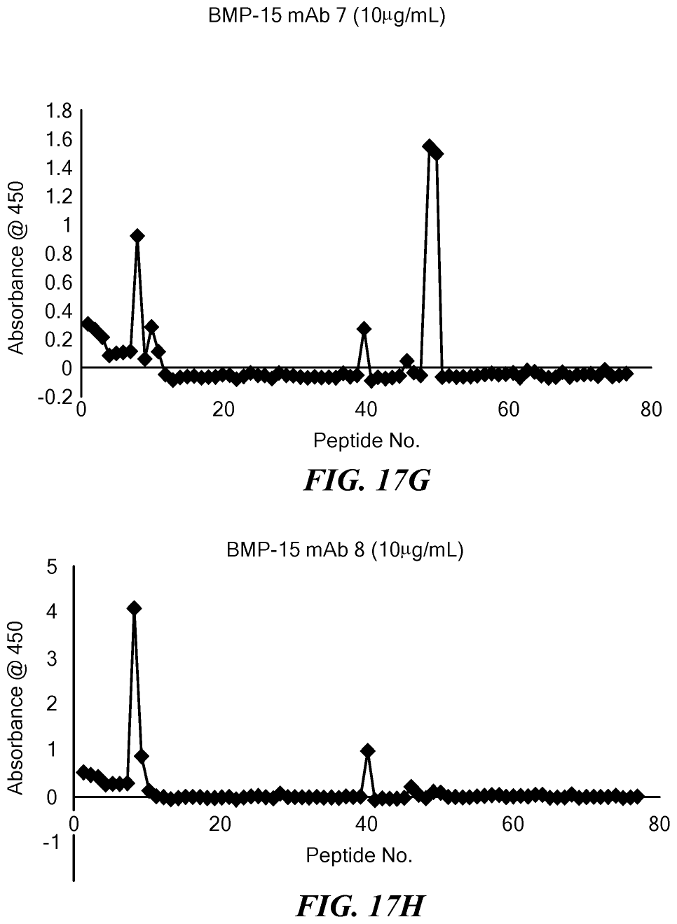

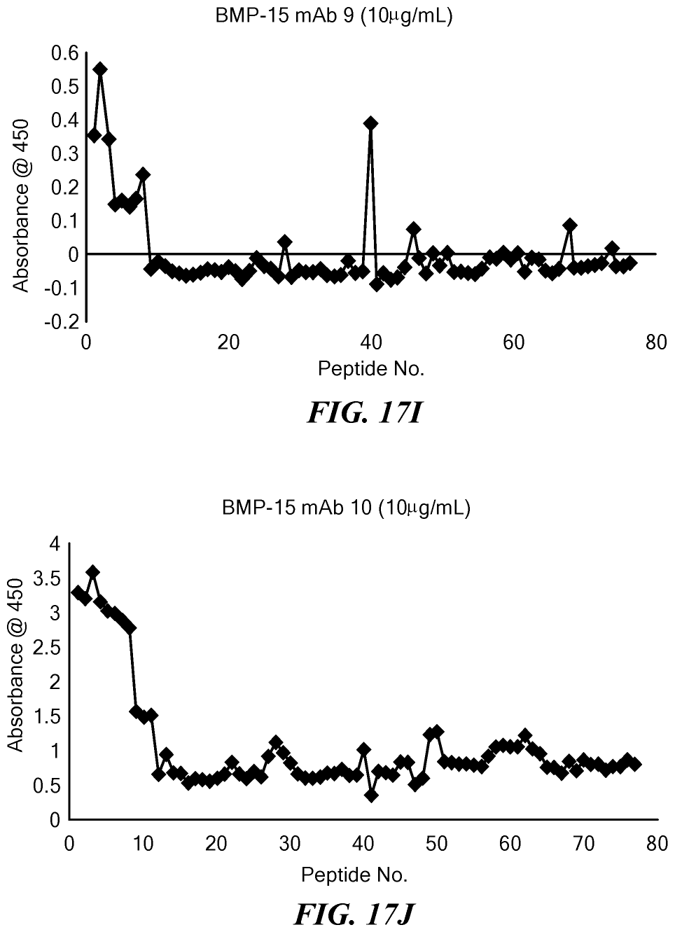

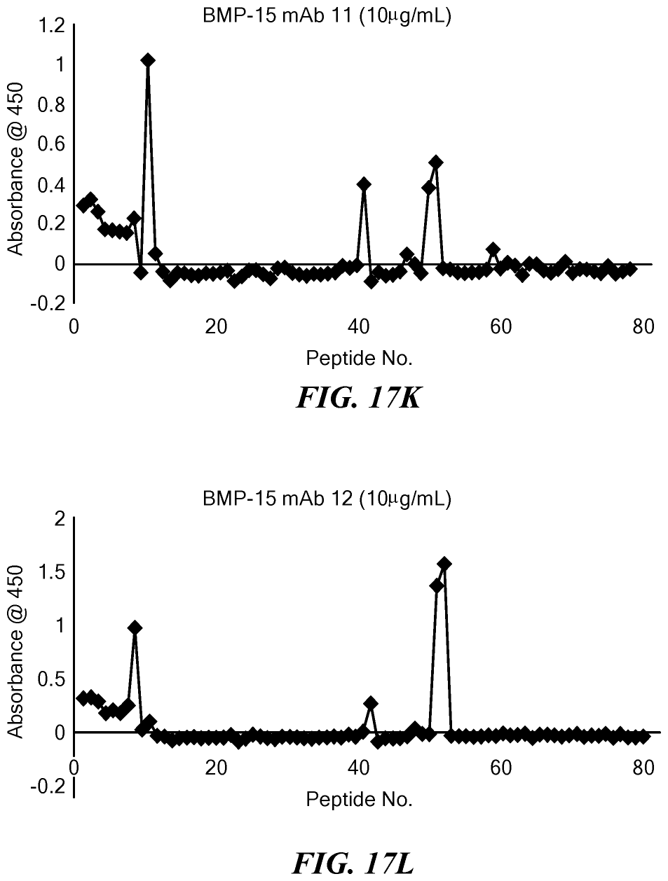

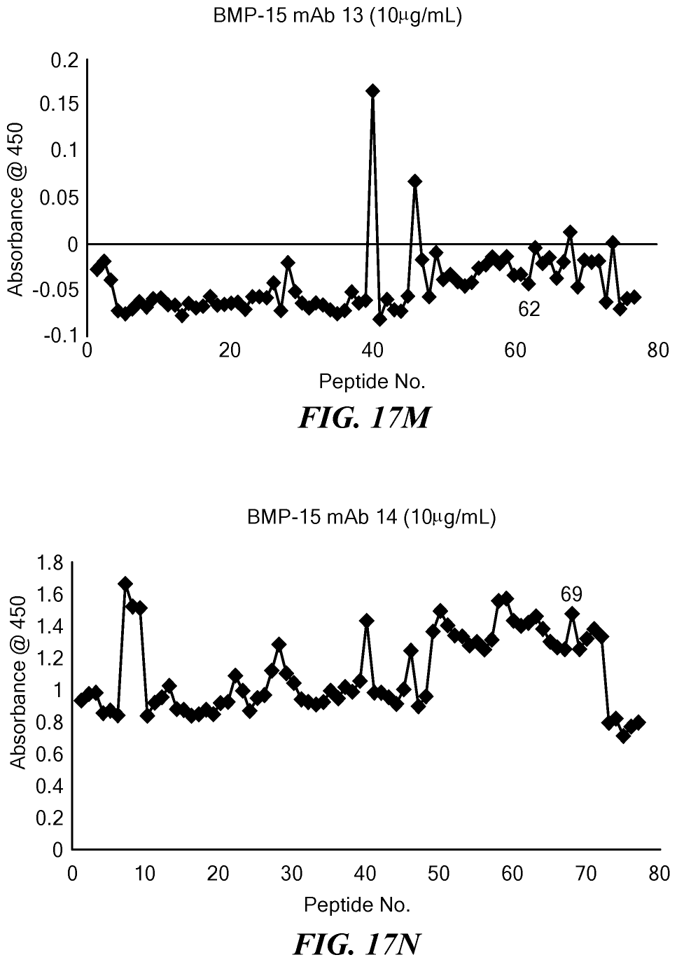

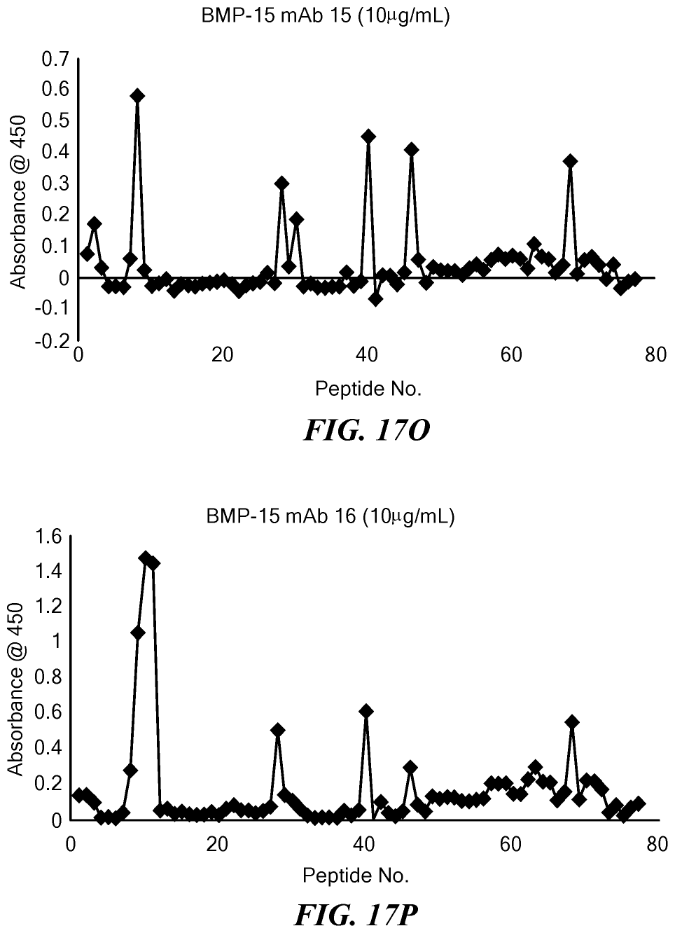

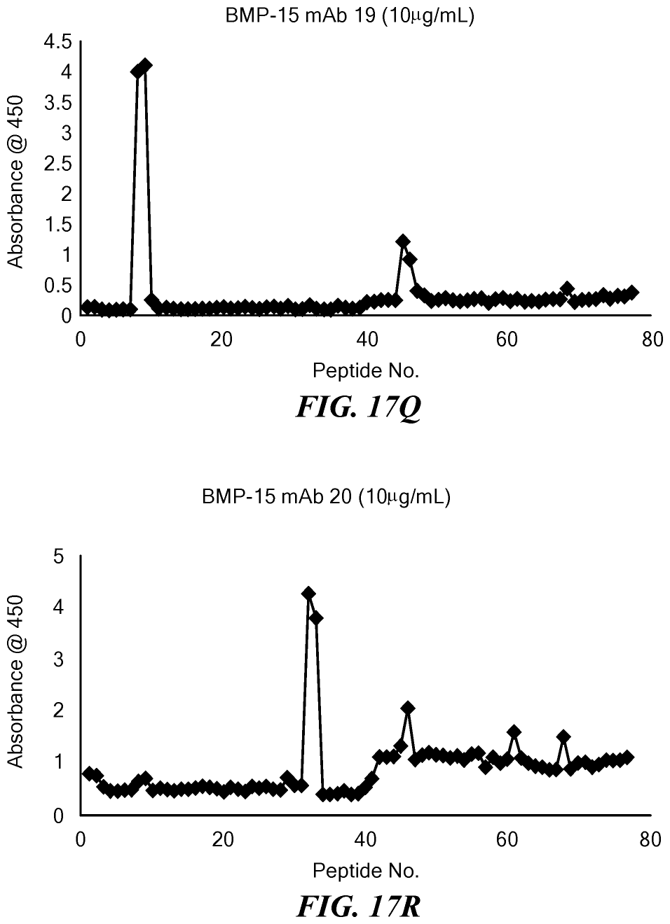

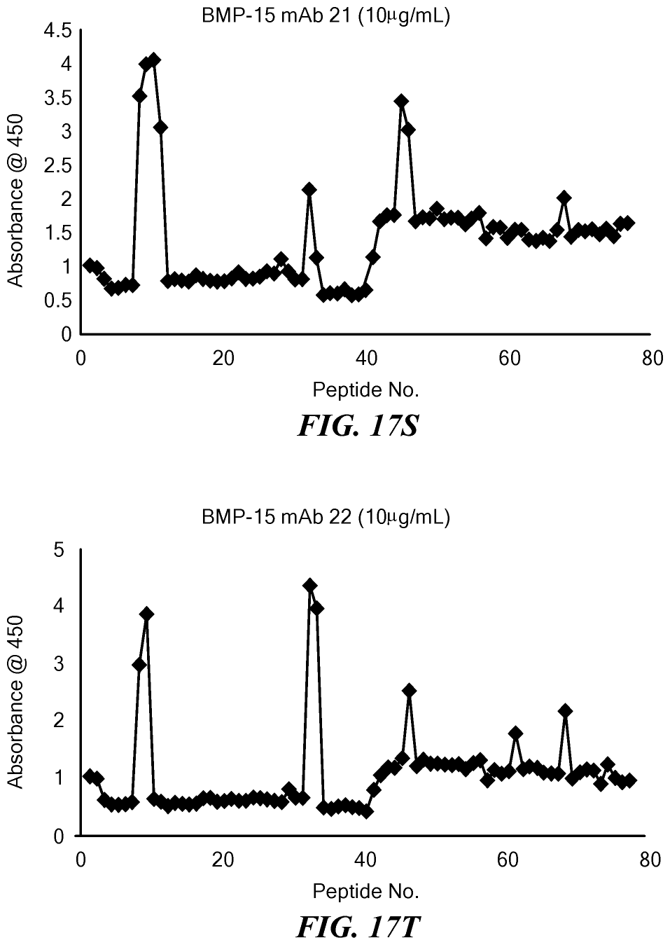

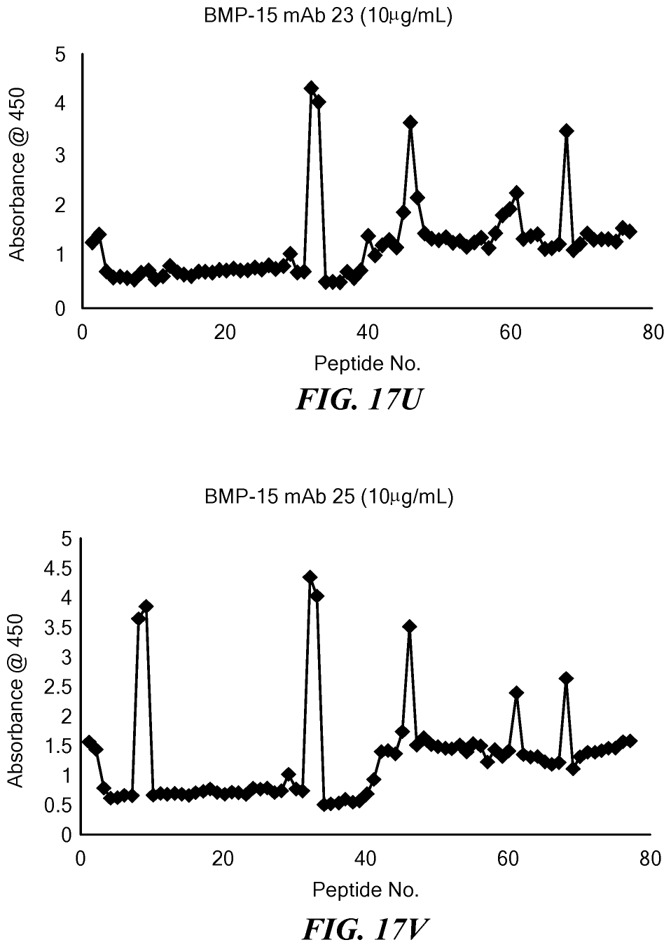

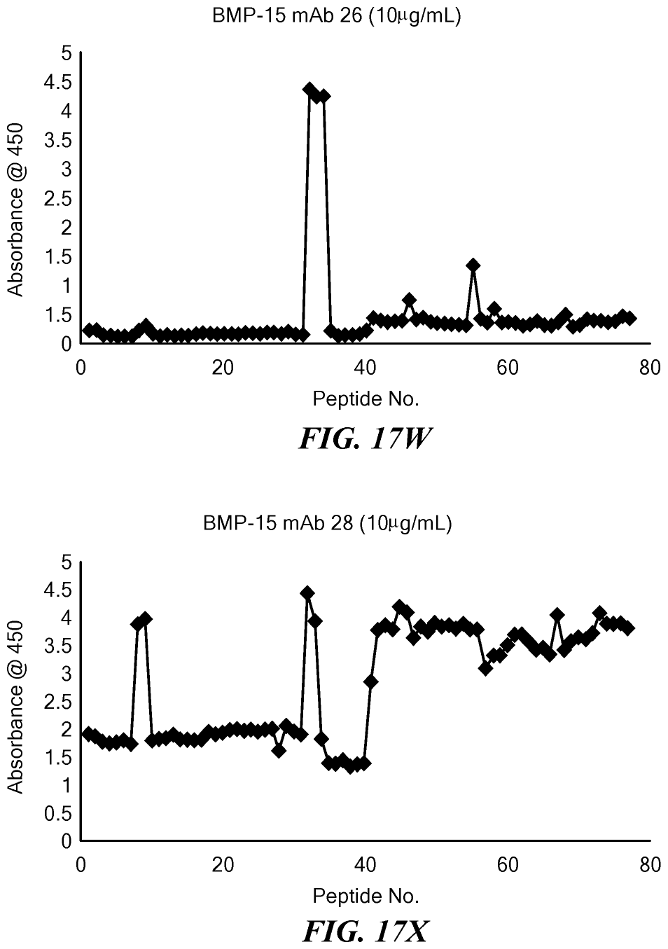

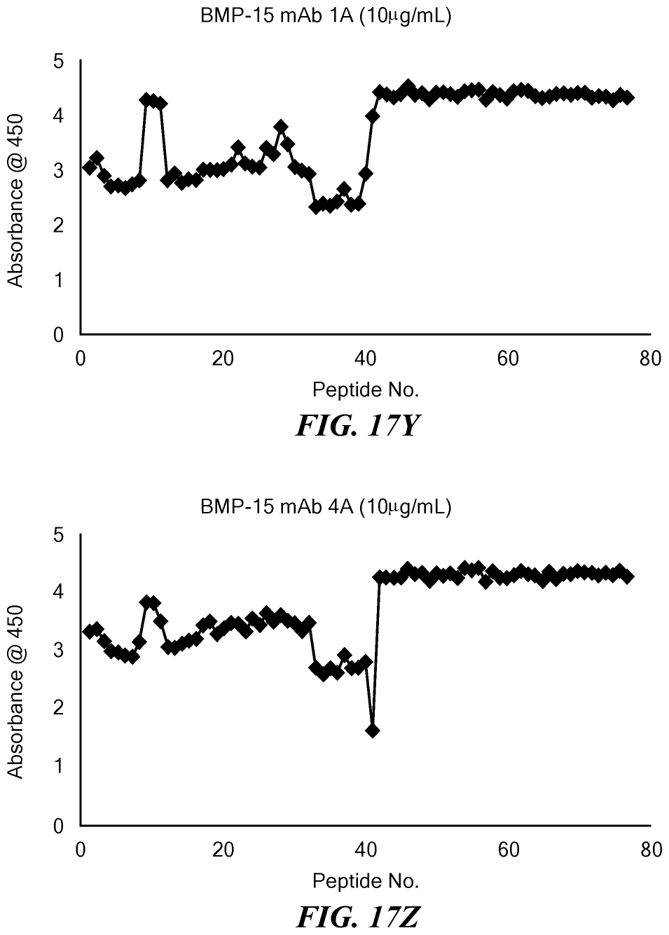

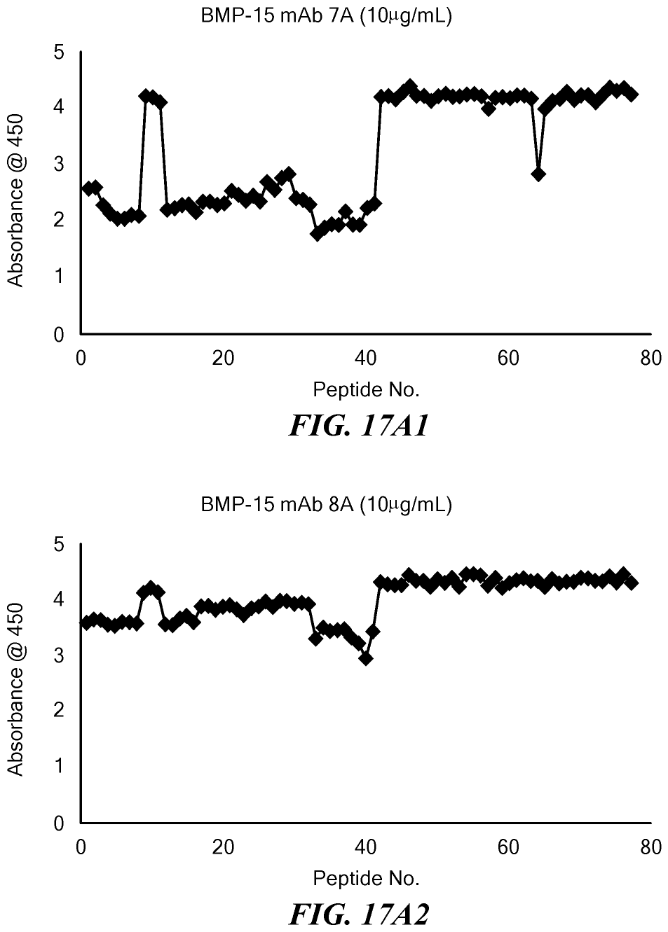

Using the above ELISA recombinant GDF-9 was detected at various concentrations and a linear relationship between concentration and detection signal was obtained (FIG. 12).

### Example 10: Validation and Characterization of BMP15 and GDF-9 mAbs with Application to Human Follicular Fluid and Human Serum

The table below summarizes results of validation and characterization of immunoassays based on BMP15 and GDF-9 mAbs described herein as applied to human follicular fluid and human serum.

| | | Capture | | | |
|---|---|---|---|---|---|
| | | 114/58A (17A) | 128/98A (23A) | 128/64 (21A) | 128/79 (25A) |
| | | Detection | | | |
| | | 98/74A (6) | 128/98A (23A) | 87/38 (#4) | 128/98A (23A) |
| | Sample | GDF-9 | BMP-15 | BMP-15 | BMP-15 − GDF-9 |
| # | ID | (ng/mL) | (pg/mL) | (pg/mL) | (ng/mL) |
| 1 | 1R | 0.342 | 2.12 | 1.87 | 0.109 |
| 2 | 2R | 0.047 | <0.402 | 1.042 | <0.053 |
| 3 | 3R | 0.00 [symbol] | 0.603 | 1.042 | <0.053 |
| 4 | 4R | 0.011 | <0.402 | 1.281 | <0.053 |
| 5 | 5R | 0.027 | 0.491 | | <0.053 |
| 6 | 7R | 0.025 | | 0.624 | <0.053 |
| 7 | 8R | 0.034 | 0.418 | 1.1 [symbol] 5 | <0.053 |
| 8 | 9R | 0.088 | 0.759 | 0.8 [symbol] 4 | <0.053 |
| 9 | 10R | 0.103 | 0.759 | 0.946 | <0.053 |
| 10 | 11R | 0.044 | 0. [symbol] 8 [symbol] | <0.3 [symbol] 8 | <0.053 |
| 11 | 12R | 0.026 | <0.402 | 0.445 | <0.053 |
| 12 | 13R | 0.07 [symbol] | 0.418 | 1.233 | <0.053 |
| 13 | 16R | 0.024 | <0.402 | 0.534 | 0.101 |
| 14 | 18R | 0.044 | 0.418 | 1.33 | <0.053 |
| 15 | 1L | 0.56 | 1.522 | 2.424 | 0.135 |
| 16 | 2L | 0.163 | 0.839 | 1.378 | 0.0 [symbol] |
| 17 | 3L | 0.038 | 0. [symbol] 28 | 0.715 | n/a |
| 18 | 4L | 0.10 [symbol] | 0.839 | 1.476 | n/a |
| 19 | 5L | 0.075 | 0.88 | 0.578 | 0.0 [symbol] 8 |
| 20 | 8L | 0.034 | 0.418 | 1.185 | 0.076 |
| 21 | 9L | 0.832 | 1.172 | 3.249 | 0.135 |
| 22 | 10L | 0.015 | 0.528 | 0.901 | 0.085 |
| 23 | 13L | 0.033 | 0.418 | 1.137 | <0.053 |
| 24 | 14L | 0.009 | 1.478 | 0.948 | n/a |
| 25 | 15L | 0.057 | 0. [symbol] 3 [symbol] | 1.233 | <0.053 |
| 26 | 16L | 0.017 | 0.491 | 0.8 [symbol] 4 | <0.053 |
| 27 | 17L | 0.073 | 0. [symbol] | 1.281 | <0.053 |
| 28 | 18L | <0.00 [symbol] | 0.491 | 1.281 | <0.053 |
| 29 | 2S | <0.005 | <0.402 | | 0.143 |
| 30 | 3S | 0.018 | 3.098 | | 0.194 |
| 31 | 4S | 0.039 | 0.921 | | <0.053 |
| 32 | 6S | 0.022 | 0. [symbol] 03 | | <0.053 |
| 33 | 7S | 0.099 | 1.258 | | 0.11 [symbol] |
| 34 | 8S | 0.104 | [symbol] . [symbol] 15 | | 0.143 |
| 35 | 9S | <0.005 | <0.402 | | <0.053 |
| 36 | 10S | 0.029 | 0.8 [symbol] | | 0.0 [symbol] 6 |
| 37 | 11S | 0.029 | 1.812 | | <0.053 |
| 38 | 12S | 0.077 | 2.263 | | 0.126 |
| 39 | 13S | <0.005 | <0.402 | | 0.126 |
| 40 | 14S | 0.023 | 8.25 | | 0.135 |
| 41 | 16S | 0.034 | 4.504 | | 0.118 |
| 42 | 17S | <0.005 | 0.5 [symbol] 5 | | 0.229 |
| 43 | 18S | 0.041 | 0. [symbol] | | 0.116 |

[symbol]

indicates data missing or illegible when filed

### Example 11: ELISA for Measuring BMP-15-GDF-9 Complex

An assay for quantifying BMP-15-GDF-9 complex under conditions in which the complex does not undergo dissociation was performed using anti-BMP-15 antibody 23A (clone 128/98A) and anti-GDF-9 antibody 25 (clone 99-69A). Several additional assays were performed for detecting one or more of BMP-15-GDF-9 complex. The antibody pairs used in all of these assays are shown in Table 11.1 below. Details of the assays are to be found in Tables 11.2 (also shown below).

TABLE 11.1

| Capture antibody (clone) | Detection antibody (clone) |
|---|---|
| anti-BMP-15 antibody 23A (128/98A) | anti-GDF-9 antibody 25A (87/38) |
| anti-BMP-15 antibody 1A (114/1) | anti-GDF-9 antibody 12A (114/5) |
| anti-BMP-15 antibody 7A (114/35) | anti-GDF-9 antibody 12A (114/5) |
| anti-GDF-9 antibody 25A (128/79) | anti-BMP-15 antibody 23A (128/98A) |
| anti-GDF-9 antibody 6 (98/74A) binding | anti-BMP-15 antibody 8A (114/40) |
| to epitopes contained in peptides having | |
| SEQ ID NOS: 123, 129, 132, 144, 145, 146 | |
| anti-GDF-9 antibody 6 (98/74A) - binding | anti-BMP-15 antibody 4A (114/16) |
| to epitopes contained in peptides having | |
| SEQ ID NOS: 123, 129, 132, 144, 145, 146 | |

TABLE 11.2

| | 1 | 2 | 3 | 1 | 2 | 3 | 4 | 5 | 6 | 7 | 8 | 9 |
|---|---|---|---|---|---|---|---|---|---|---|---|---|
| A | Act A | GDF-9 | Bovine FF | 0.138 | 0.118 | 0.14 | 0.154 | 0.159 | 0.129 | 0.079 | 0.091 | 0.066 |
| | Matrix | (Mature) | 1 1:2 | | | | | | | | | |
| | | 1 ng/mL | | | | | | | | | | |
| B | Complex | Human FF | Equine | 0.393 | 0.124 | 0.124 | 0.195 | 0.108 | 0.142 | 0.109 | 0.05 | 0.066 |
| | 1:40000 | 2 neat | pool | | | | | | | | | |
| | | | neat | | | | | | | | | |
| C | Complex | Human FF | Equine | 0.508 | 0.122 | 0.114 | 0.225 | 0.123 | 0.155 | 0.126 | 0.053 | 0.065 |
| | 1:25000 | 2 1:2 | pool | | | | | | | | | |
| | | | 1:2 | | | | | | | | | |
| D | Complex | Human FF | Canine | 0.905 | 0.124 | 0.152 | 0.288 | 0.115 | 0.159 | 0.192 | 0.056 | 0.083 |
| | 1:10000 | 3 neat | ext | | | | | | | | | |
| | | | 1:10 | | | | | | | | | |
| E | Complex | Human FF | Canine | 3.724 | 0.12 | 0.126 | 1.023 | 0.12 | 0.163 | 0.875 | 0.053 | 0.085 |
| | 1:2000 | 3 1:2 | ext | | | | | | | | | |
| | | | 1:20 | | | | | | | | | |
| F | BMP-15 | Human FF | Mouse | 4.357 | 0.121 | 0.124 | 0.493 | 0.1 | 0.181 | 0.515 | 0.037 | 0.079 |
| | (P + M) | A4 neat | Testis | | | | | | | | | |
| | (5ng/mL) | | ext 1:10 | | | | | | | | | |
| G | BMP-15 | Human FF | Mouse | 4.275 | 0.127 | 0.127 | 0.157 | 0.111 | 0.163 | 0.084 | 0.055 | 0.075 |
| | (Mature) | A4 1:2 | Testis | | | | | | | | | |
| | 1 ng/ml | | ext 1:20 | | | | | | | | | |
| H | GDF-9 | Bovine FF | | 0.16 | 0.139 | | 0.159 | 0.107 | | 0.075 | 0.047 | |
| | (P + M) | 1 neat | | | | | | | | | | |
| | (5 ng/mL) | | | | | | | | | | | |

| | | | |
|---|---|---|---|
| Capture | 128/98 (#23A) | 114/1 (#1A) | 114/35 (#7A) |
| BMP-15 | | | |
| Detection | 128/79 (#25A) | 114/5 (#12A) | 114/5 (#12A) |
| GDF-9 | | | |

| Plt 2 | 1 | 2 | 3 | 1 | 2 | 3 | 4 | 5 | 6 | 7 | 8 | 9 |
|---|---|---|---|---|---|---|---|---|---|---|---|---|
| A | Act A | GDF-9 | Bovine | 0.042 | 0.038 | 0.032 | 0.054 | 0.053 | 0.047 | 0.057 | 0.047 | 0.041 |
| | Matrix | (Mature) | FF | | | | | | | | | |
| | | 1 ng/mL | 1 1:2 | | | | | | | | | |
| B | Complex | Human | Equine | 0.288 | 0.04 | 0.03 | 0.048 | 0.04 | 0.084 | 0.047 | 0.037 | 0.08 |
| | 1:40000 | FF | pool | | | | | | | | | |
| | | 2 neat | neat | | | | | | | | | |
| C | Complex | Human | Equine | 0.395 | 0.038 | 0.033 | 0.056 | 0.042 | 0.074 | 0.049 | 0.041 | 0.072 |
| | 1.25000 | FF | pool | | | | | | | | | |
| | | 2 1:2 | 1:2 | | | | | | | | | |
| D | Complex | Human | Canine | 0.953 | 0.032 | 0.055 | 0.054 | 0.046 | 0.072 | 0.046 | 0.042 | 0.079 |
| | 1:10000 | FF | ext | | | | | | | | | |
| | | 3 neat | 1:10 | | | | | | | | | |
| E | Complex | Human | Canine | 3.607 | 0.037 | 0.042 | 0.069 | 0.047 | 0.077 | 0.048 | 0.044 | 0.077 |
| | 1:2000 | FF | ext | | | | | | | | | |
| | | 3 1:2 | 1:20 | | | | | | | | | |
| F | BMP-15 | Human | Mouse | 4.363 | 0.034 | 0.048 | 0.056 | 0.046 | 0.106 | 0.046 | 0.036 | 0.095 |
| | (P + M) | FF | Testis | | | | | | | | | |
| | (5 ng/mL) | A4 neat | ext 1:10 | | | | | | | | | |
| G | BMP-15 | Human | Mouse | 4.298 | 0.03 | 0.04 | 0.054 | 0.046 | 0.081 | 0.046 | 0.034 | 0.095 |
| | (Mature) | FF | Testis | | | | | | | | | |
| | 1 ng/mL | A4 1:2 | ext 1:20 | | | | | | | | | |
| H | GDF-9 | Bovine | | 0.048 | 0.041 | | 0.065 | 0.057 | | 0.062 | 0.043 | |
| | (P + M) | FF | | | | | | | | | | |
| | (5 ng/mL) | 1 neat | | | | | | | | | | |

| | | | |
|---|---|---|---|
| Capture | 128/79 (#25A) | 98/74 (#6) | 98/74 (#6) |
| GDF-9 | | | |
| Detection | 128/98 (#23A) | 114/40 (#8A) | 114/16 (#4A) |
| BMP-15 | | | |

| | O.D. | O.D. | |
|---|---|---|---|
| Well ID | (Rep.) | (Mean) | % CV |
| BLK | 0.020 | 0.019 | 7.443 |
| 0.018 | 0.018 | | |

### Complex Ollie, BB023, Lot #12.5.2014

| | O.D. | O.D. | | Conc. | Conc. | Pred. Conc. |
|---|---|---|---|---|---|---|
| Well ID | (Rep.) | (Mean) | % CV | (pg/mL) | (Rep.) | (Mean) |
| STD1 | 0.044 | 0.046 | 4.662 | 0.356 | 0.371 | 0.385 |
| | 0.047 | | | 0.356 | 0.398 | |
| STD2 | 0.072 | 0.074 | 3.822 | 0.712 | 0.627 | 0.646 |
| | 0.076 | | | 0.712 | 0.664 | |
| STD3 | 0.147 | 0.147 | 0.483 | 1.346 | 1.342 | 1.337 |
| | 0.146 | | | 1.346 | 1.332 | |
| STD4 | 0.281 | 0.283 | 0.999 | 2.748 | 2.677 | 2.698 |
| | 0.285 | | | 2.748 | 2.718 | |
| STD5 | 0.555 | 0.549 | 1.416 | 5.401 | 5.530 | 5.472 |
| | 0.544 | | | 5.401 | 5.413 | |
| STD6 | 1.080 | 1.065 | 2.059 | 10.611 | 11.244 | 11.072 |
| | 1.049 | | | 10.611 | 10.900 | |
| STD7 | 1.925 | 1.934 | 0.622 | 20.345 | 20.819 | 20.917 |
| | 1.942 | | | 20.345 | 21.015 | |
| STD8 | 3.527 | 3.485 | 1.704 | 40.69 | 39.697 | 39.193 |
| | 3.443 | | | 40.69 | 38.69 | |

### Example 13: ELISA for Measuring Pro+Mature GDF-9, Mature GDF-9, and BMP-15-GDF-9 Complex Using Anti-GDF-9 Antibodies

A number of assays, each using anti-GDF-9 antibodies were performed (see Tables 13.2 through 13.5 below). Three types of assay buffers, namely (i) ASB-205, (ii) CND-123, and (iii) a combination ASB-205 and CND-123 were used for the assays. Further, assays using the ASB-205 and CND-123 combined buffer system were carried out both in the absence and in the presence of protein dissociating agents SDS and Triton-X 100. Table 13.1 below lists the antibody pairs used for each assay.

TABLE 13.1

| Capture: anti-GDF-9 antibody (clone) | Detection: anti-GDF-9 antibody (clone) - epitope |
|---|---|
| 12A (114/5A) | 6 (98/74A) - binding to epitopes contained in |
| | peptides having SEQ ID NOS: 123, 129, 132, |
| | 144, 145, and 146 |
| 13A (114/29A) - binding to epitopes | 6 (98/74A) - binding to epitopes contained in |
| contained in peptides having SEQ ID | peptides having SEQ ID NOS: 123, 129, 132, |
| NOS: 111, 123, and 132 | 144, 145, and 146 |
| 14A (114/31A) - | 6 (98/74A) - binding to epitopes contained in |
| | peptides having SEQ ID NOS: 123, 129, 132, |
| | 144, 145, and 146 |
| 17A (114/58A) - binding to epitopes | 6 (98/74A) - binding to epitopes contained in |
| contained in peptides having SEQ ID | peptides having SEQ ID NOS: 123, 129, 132, |
| NOS: 123, 135, and 136 | 144, 145, and 146 |
| 25 (99/69A) | 6 (98/74A) - binding to epitopes contained in |
| | peptides having SEQ ID NOS: 123, 129, 132, |
| | 144, 145, and 146 |
| 25 (99/69A) | 3 (98/80A) - binding to epitopes contained in |
| | peptides having SEQ ID NOS: 123, 124, 132, |
| | and 133 |
| 6 (98/74A) - binding to epitopes contained | 12A (114/5A) |
| in peptides having SEQ ID NOS: 123, 129, | |
| 132, 144, 145, and 146 | |
| 6 (98/74A) - binding to epitopes contained | 16A (114/50A) |
| in peptides having SEQ ID NOS: 123, 129, | |
| 132, 144, 145, and 146 | |
| 6 (98/74A) - binding to epitopes contained | 17A (114/58A) - binding to epitopes contained in |
| in peptides having SEQ ID NOS: 123, 129, | peptides having SEQ ID NOS: 123, 135, and 136 |
| 132, 144, 145, and 146 | |

The dissociating agents were used in the concentrations indicated in Tables 13.3 and 13.4.

TABLE 13.2
Table 13.2

| | | | BMP-15/ | BMP-15/ | GDF-9 | GDF-9 | GDF-9 | GDF-9 | GDF-9 |
|---|---|---|---|---|---|---|---|---|---|
| | | | GDF-9 | GDF-9 | p + m | p + m | p + m | mature | mature |
| | | Act A | Complex | Complex | pur. 11 | pur. 110 | pur. 1100 | 100 | 1000 |
| Capture | Detection | matrix | 1:500000 | 1:10000 | pg/mL | pg/mL | pg/mL | pg/mL | pg/mL |
| 12A | 6 | 0.067 | 0.124 | 2.121 | 0.088 | 0.246 | 1.808 | 0.304 | 3.152 |
| 13A | 6 | 0.038 | 0.097 | 2.163 | 0.066 | 0.274 | 2.335 | 0.293 | 3.394 |
| 14A | 6 | 0.041 | 0.099 | 2.025 | 0.065 | 0.243 | 1.937 | 0.289 | 3.79 |
| 17A | 6 | 0.037 | 0.11 | 2.613 | 0.06 | 0.26 | 2.244 | 0.319 | 3.467 |
| 25 | 6 | 0.033 | 0.031 | 0.207 | 0.038 | 0.107 | 0.857 | 0.023 | 0.038 |
| 25 | 3 | 0.046 | 0.042 | 0.271 | 0.049 | 0.133 | 1.062 | 0.035 | 0.045 |
| 6 | 12A | 0.697 | 0.556 | 3.531 | 0.413 | 0.582 | 2.666 | 0.713 | 3.485 |
| 6 | 16A | 0.054 | 0.073 | 0.983 | 0.051 | 0.111 | 0.644 | 0.135 | 1.21 |
| 6 | 17A | 0.031 | 0.048 | 0.719 | 0.036 | 0.109 | 0.802 | 0.102 | 1.144 |
| | | ASB-205 as Assay Buffer | | | | | | | |
| 12A | 6 | 0.058 | 0.155 | 2.533 | 0.077 | 0.227 | 1.587 | 0.226 | 1.97 |
| 13A | 6 | 0.035 | 0.102 | 2.876 | 0.059 | 0.259 | 2.255 | 0.245 | 2.299 |
| 14A | 6 | 0.037 | 0.101 | 2.527 | 0.054 | 0.203 | 1.739 | 0.218 | 2.384 |
| 17A | 6 | 0.028 | 0.107 | 3.037 | 0.053 | 0.241 | 2.179 | 0.267 | 2.477 |
| 25 | 6 | 0.024 | 0.024 | 0.157 | 0.031 | 0.09 | 0.712 | 0.024 | 0.025 |
| 25 | 3 | 0.038 | 0.034 | 0.202 | 0.04 | 0.125 | 0.985 | 0.035 | 0.043 |
| 6 | 12A | 0.556 | 0.645 | 3.88 | 0.531 | 0.736 | 2.785 | 0.709 | 3.662 |
| 6 | 16A | 0.052 | 0.078 | 1.345 | 0.056 | 0.104 | 0.639 | 0.132 | 1.359 |
| 6 | 17A | 0.027 | 0.044 | 0.915 | 0.034 | 0.089 | 0.727 | 0.084 | 1.016 |

CND-123 as Assay Buffer

| | | | | | | | | | Mouse |
|---|---|---|---|---|---|---|---|---|---|
| | | Human | Human | Human | Human | Bovine | Equine | Canine | Testis |
| | | FF A3 | FF A3 | FF A4 | FF A4 | FF 1 | 1/2 cent | Ext | Ext. pool |
| Capture | Detection | 1/2 | neat | 1/2 | neat | neat | pool | 1:4 | 1:10 |
| 12A | 6 | 0.054 | 0.075 | 0.057 | 0.073 | 0.07 | 0.062 | 0.073 | 0.085 |
| 13A | 6 | 0.055 | 0.068 | 0.052 | 0.067 | 0.074 | 0.038 | 0.063 | 0.063 |
| 14A | 6 | 0.059 | 0.071 | 0.053 | 0.065 | 0.072 | 0.04 | 0.054 | 0.081 |
| 17A | 6 | 0.056 | 0.077 | 0.056 | 0.071 | 0.078 | 0.036 | 0.05 | 0.051 |
| 25 | 6 | 0.029 | 0.026 | 0.03 | 0.022 | 0.022 | 0.027 | 0.039 | 0.084 |
| 25 | 3 | 0.048 | 0.035 | 0.038 | 0.034 | 0.037 | 0.034 | 0.055 | 0.108 |
| 6 | 12A | 0.378 | 0.313 | 0.313 | 0.291 | 0.297 | 1.382 | 0.273 | 0.589 |
| 6 | 16A | 0.053 | 0.053 | 0.052 | 0.047 | 0.059 | 0.08 | 0.081 | 0.193 |
| 6 | 17A | 0.033 | 0.036 | 0.028 | 0.032 | 0.036 | 0.034 | 0.062 | 0.16 |
| | | ASB-205 as Assay Buffer | | | | | | | |
| 12A | 6 | 0.063 | 0.089 | 0.063 | 0.087 | 0.085 | 0.043 | 0.063 | 0.076 |
| 13A | 6 | 0.058 | 0.084 | 0.062 | 0.082 | 0.083 | 0.032 | 0.047 | 0.061 |
| 14A | 6 | 0.059 | 0.083 | 0.058 | 0.078 | 0.085 | 0.033 | 0.051 | 0.057 |
| 17A | 6 | 0.064 | 0.091 | 0.065 | 0.089 | 0.091 | 0.031 | 0.04 | 0.045 |
| 25 | 6 | 0.025 | 0.021 | 0.028 | 0.02 | 0.024 | 0.024 | 0.034 | 0.1 |
| 25 | 3 | 0.039 | 0.036 | 0.038 | 0.034 | 0.037 | 0.032 | 0.046 | 0.123 |
| 6 | 12A | 0.474 | 0.414 | 0.411 | 0.392 | 0.379 | 1.241 | 0.313 | 0.584 |
| 6 | 16A | 0.058 | 0.062 | 0.054 | 0.058 | 0.06 | 0.076 | 0.069 | 0.219 |
| 6 | 17A | 0.037 | 0.037 | 0.032 | 0.038 | 0.036 | 0.038 | 0.047 | 0.181 |

CND-123 as Assay Buffer

TABLE 13.3

| | | | BMP-15/ | BMP-15/ | GDF-9 | GDF-9 | GDF-9 | GDF-9 | GDF-9 |
|---|---|---|---|---|---|---|---|---|---|
| | | | GDF-9 | GDF-9 | p + m | p + m | p + m | mature | mature |
| | | Act A | Complex | Complex | pur. 11 | pur. 110 | pur. 1100 | 100 | 1000 |
| Capture | Detection | matrix | 1:500000 | 1:10000 | pg/mL | pg/mL | pg/mL | pg. | pg/mL |
| 12A | 6 | 0.07 | 0.116 | 2.524 | 0.067 | 0.106 | 0.483 | 0.108 | 0.518 |
| 13A | 6 | 0.042 | 0.12 | 3.176 | 0.045 | 0.127 | 0.918 | 0.101 | 0.733 |
| 14A | 6 | 0.045 | 0.108 | 2.899 | 0.04 | 0.087 | 0.505 | 0.078 | 0.499 |
| 17A | 6 | 0.038 | 0.118 | 3.465 | 0.049 | 0.104 | 0.809 | 0.106 | 0.785 |
| 25 | 6 | 0.034 | 0.028 | 0.144 | 0.026 | 0.046 | 0.243 | 0.029 | 0.027 |
| 25 | 3 | 0.049 | 0.044 | 0.287 | 0.045 | 0.124 | 0.985 | 0.039 | 0.045 |
| 6 | 12A | 0.83 | 0.788 | 2.595 | 0.624 | 0.73 | 1.463 | 0.745 | 1.467 |
| 6 | 16A | 0.063 | 0.055 | 0.512 | 0.062 | 0.069 | 0.271 | 0.073 | 0.261 |
| 6 | 17A | 0.034 | 0.039 | 0.428 | 0.035 | 0.059 | 0.28 | 0.049 | 0.218 |

ASB-123A + CND-123 (0.125% SDS, 1% Triton) as Assay Buffer

| | | | | | | | | | Mouse |
|---|---|---|---|---|---|---|---|---|---|
| | | Human | Human | Human | Human | Bovine | Equine | Canine | Testis |
| | | FF A3 | FF A3 | FF A4 | FF A4 | FF 1 | 1/2 cent | Ext | Ext. pool |
| Capture | Detection | 1/2 | neat | 1/2 | neat | neat | pool | 1:4 | 1:10 |
| 12A | 6 | 0.067 | 0.088 | 0.069 | 0.089 | 0.091 | 0.047 | 0.091 | 0.234 |
| 13A | 6 | 0.062 | 0.08 | 0.06 | 0.076 | 0.086 | 0.034 | 0.066 | 0.25 |
| 14A | 6 | 0.061 | 0.085 | 0.063 | 0.078 | 0 085 | 0.039 | 0.064 | 0.249 |
| 17A | 6 | 0.07 | 0.093 | 0.065 | 0.099 | 0.091 | 0.037 | 0.061 | 0.232 |
| 25 | 6 | 0.028 | 0.025 | 0.028 | 0.024 | 0.027 | 0.034 | 0.056 | 0.315 |
| 25 | 3 | 0.049 | 0.046 | 0.044 | 0.042 | 0.053 | 0.053 | 0.205 | 1.333 |
| 6 | 12A | 0.673 | 0.513 | 0.521 | 0.436 | 0.479 | 1.524 | 1.052 | 3.601 |
| 6 | 16A | 0.059 | 0.055 | 0.061 | 0.059 | 0.059 | 0.063 | 0.089 | 0.573 |
| 6 | 17A | 0.037 | 0.041 | 0.041 | 0.037 | 0.042 | 0.048 | 0.054 | 0.172 |

ASB-123A + CND-123 (0.125% SDS, 1% Triton) as Assay Buffer

TABLE 13.4

| | | | BMP-h5/ | BMP-15/ | GDF-9 | GDF-9 | GDF-9 | GDF-9 | GDF-9 |
|---|---|---|---|---|---|---|---|---|---|
| | | | GDF-9 | GDF-9 | p + m | p + m | p + m | mature | mature |
| | | Act A | Complex | Complex | pur. 11 | pur. 110 | pur. 1100 | 100 | 1000 |
| Capture | Detection | matrix | 1:500000 | 1:10000 | pg/mL | pg/mL | pg/mL | pg/mL | pg/mL |
| 12A | 6 | 0.057 | 0.128 | 2.815 | 0.076 | 0.148 | 0.985 | 0.125 | 0.818 |
| 13A | 6 | 0.041 | 0.126 | 3.139 | 0.055 | 0.21 | 1.657 | 0.13 | 1.062 |
| 14A | 6 | 0.044 | 0.121 | 2.833 | 0 052 | 0.157 | 1.229 | 0.119 | 1.048 |
| 17A | 6 | 0.041 | 0.123 | 3.513 | 0.053 | 0.176 | 1.572 | 0.14 | 1.139 |
| 25 | 6 | 0.032 | 0.023 | 0.191 | 0.048 | 0.075 | 0.491 | 0.024 | 0.029 |
| 25 | 3 | 0.043 | 0.05 | 0.255 | 0.046 | 0.136 | 0.986 | 0.042 | 0.045 |
| 6 | 12A | 0.691 | 0.829 | 4.011 | 0.659 | 0.837 | 2.312 | 0.739 | 2.35 |
| 6 | 16A | 0.057 | 0.082 | 1.151 | 0.062 | 0.095 | 0.439 | 0.092 | 0.528 |
| 6 | 17A | 0044 | 0055 | 0.822 | 0.051 | 0.101 | 0.522 | 0.063 | 0.453 |

ASB-123A + CND-123 (0.125% SDS, 1% Triton) as Assay Buffer

| | | | | | | | | | Mouse |
|---|---|---|---|---|---|---|---|---|---|
| | | Human | Human | Human | Human | Bovine | Equine | Canine | Testis |
| | | FF A3 | FF A3 | FF A4 | FF A4 | FF 1 | 1/2 cent | Ext | Ext. pool |
| Capture | Detection | 1/2 | neat | 1/2 | neat | neat | pool | 1:4 | 1:10 |
| 12A | 6 | 0.079 | 0.091 | 0.073 | 0.09 | 0.09 | 0.048 | 0.067 | 0.086 |
| 13A | 6 | 0.06 | 0.081 | 0.063 | 0.081 | 0.085 | 0.037 | 0.052 | 0.095 |
| 14A | 6 | 0.067 | 0.086 | 0 065 | 0.081 | 0.089 | 0.037 | 0.049 | 0.078 |
| 17A | 6 | 0.066 | 0.099 | 0.065 | 0.094 | 0.075 | 0.033 | 0.044 | 0.095 |
| 25 | 6 | 0.029 | 0.029 | 0.034 | 0.024 | 0.034 | 0.032 | 0.048 | 0.137 |
| 25 | 3 | 0.044 | 0.043 | 0.044 | 0.041 | 0.061 | 0.043 | 0.08 | 0.2 |
| 6 | 12A | 0.57 | 0.494 | 0.493 | 0.446 | 0.447 | 1.49 | 0.528 | 1.06 |
| 6 | 16A | 0.056 | 0.059 | 0.057 | 0.056 | 0.056 | 0.096 | 0.077 | 0.178 |
| 6 | 17A | 0.046 | 0.044 | 0.041 | 0.04 | 0.042 | 0.043 | 0.051 | 0.115 |

ASB-123A + CND-123 (0.125% SDS, 1% Triton) as Assay Buffer

A sequence listing is provided as an ASCII text file named “Sequence-Listing-25Mar2021-12279-0703” created on 25 Mar. 2021 and having a size of 33339 bytes. The ASCII text file is hereby incorporated by reference in the application.

### REFERENCES

1. Persani L, Rossetti R, Di Pasquale E, Cacciatore C, Fabre S. The fundamental role of bone morphogenetic protein 15 in ovarian function and its involvement in female fertility disorders. Hum Reprod Update. 2014 Jun. 30. [Epub ahead of print] Review.
2. Dube, J. L. et al. (1998) Mol. Endocrinol. 12:1809.
3. Saito, S. et al. (2008) Prot. Sci. 17:362.
4. McMahon, H. E. et al. (2008) Endocrinology 149:812.
5. Moore, R. K. et al. (2003) J. Biol. Chem. 278:304.
6. Edwards, S. J. et al. (2008) Endocrinology 149:1026.
7. Liao, W. X. et al. (2003) J. Biol. Chem. 278:3713.
8. McNatty K P, Lawrence S, Groome N P, Meerasahib M F, Hudson N L, Whiting L, Heath D A, Juengel J L. Meat and Livestock Association Plenary Lecture 2005. Oocyte signaling molecules and their effects on reproduction in ruminants. Reprod Fertil Dev. 2006; 18:403-12.
9. Lin J Y, Pitman-Crawford J L, Bibby A H, Hudson N L, McIntosh C J, Juengel J L, McNatty K P. Effects of species differences on oocyte regulation of granulosa cell function. Reproduction. 2012; 144:557-67.
10. Wu Y T, Wang T T, Chen X J, Zhu X M, Dong M Y, Sheng J Z, Xu C M, Huang H F. Bone morphogenetic protein-15 in follicle fluid combined with age may differentiate between successful and unsuccessful poor ovarian responders. Reprod Biol Endocrinol. 2012 10:116.
11. Wu Y T, Tang L, Cai J, Lu X E, Xu J, Zhu X M, Luo Q, Huang H F. High bone morphogenetic protein-15 level in follicular fluid is associated with high quality oocyte and subsequent embryonic development. Hum Reprod. 2007 22:1526-31.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method of quantifying GDF-9-BMP-15 heterodimers in a sample, the method comprising:
performing an immunoassay on the sample using a first antibody that specifically binds to an epitope of GDF-9 and a second antibody that specifically binds to an epitope of BMP-15, wherein one of said first and second antibodies is used for capture and the other is used for detection step of the immunoassay;
measuring a detection signal generated by an agent conjugated to the detection antibody; and
quantifying the amount of GDF-9-BMP-15 heterodimers in the sample by comparing the detection signal to a calibration curve correlating an amount of GDF-9-BMP-15 heterodimer to the detection signal.

2. The method of claim 1, wherein the immunoassay is a sandwich ELISA.

3. The method of claim 1, wherein the step of measuring a detection signal comprises measuring a fluorescence signal or a chemiluminiscence signal.

4.-6. (canceled)

7. The method of claim 1, wherein the sample is follicular fluid.

8. The method of claim 1, wherein the immunoassay is performed in the presence of one or more protein dissociating agents.

9. The method of claim 8, wherein the one or more protein dissociating agents are selected from the group consisting of Triton-X 100, guanidinium chloride, sodium dodecyl sulfate (SDS), urea, thiourea, lithium perchlorate, lithium acetate, and magnesium chloride.

10.-12. (canceled)

13. The method of claim 1, wherein the first antibody specifically binds to an epitope of GDF-9 contained in an amino acid sequence selected from SEQ ID NOS: 111, 119, 120, 123-125, 128-136, and 144-146; and wherein the second antibody binds to an epitope of BMP-15 contained in an amino acid sequence selected from SEQ ID NOS: 8-12, 28, 30, 32-34, 40, 41, 45, 46, 48-51, 55, 61, and 68.

14. The method of claim 13, wherein the first antibody specifically binds to an epitope of GDF-9 contained in an amino acid sequence selected from SEQ ID NOS: 111, 119, 120, 123-125, 128-136, and 144-146; and the second antibody binds to an epitope of BMP-15 contained in an amino acid sequence selected from SEQ ID NOS: 8, 9, 12, 32, and 33.

15. A method of quantifying BMP-15 homodimer in a sample, the method comprising performing an immunoassay on the sample using the same anti-BMP-15 antibody for both capture and detection steps of the immunoassay, wherein the antibody specifically binds an epitope contained in an amino acid sequence selected from SEQ ID NOS: 8-12, 28, 30, 32-34, 40, 41, 45, 46, 48-51, 55, 61, and 68;
measuring a detection signal generated by an agent conjugated to the anti-BMP-15 antibody used for detection step; and
quantifying the amount of BMP-15 homodimer in the sample by comparing the detection signal to a calibration curve correlating an amount of BMP-15 homodimer to the detection signal.

16. The method of claim 15, wherein the immunoassay is a sandwich ELISA.

17. The method of claim 15, wherein the measuring a detection signal comprises measuring a fluorescence signal or a chemiluminiscence signal.

18. (canceled)

19. The method of claim 15, wherein the antibody specifically binds an epitope contained in an amino acid sequence selected from SEQ ID NOS: 8 and 9.

20. The method of claim 15, wherein the antibody specifically binds an epitope contained in an amino acid sequence selected from SEQ ID NOS: 32 and 33.

21. A method of quantifying GDF-9 homodimer in a sample, the method comprising:
performing an immunoassay on the sample using the same anti-GDF-9 antibody for both capture and detection steps of the immunoassay, wherein the antibody specifically binds an epitope contained in an amino acid sequence selected from SEQ ID NOS: 111, 119, 120, 123-125, 128-136, and 144-146;
measuring a detection signal generated by an agent conjugated to the anti-GDF-9 antibody used for detection step; and
quantifying the amount of GDF-9 homodimer in the sample by comparing the detection signal to a calibration curve correlating an amount of GDF-9 homodimer to the detection signal.

22. The method of claim 21, wherein the immunoassay is a sandwich ELISA.

23. The method of claim 21, wherein the measuring a detection signal comprises measuring a fluorescence signal or a chemiluminiscence signal.

24. (canceled)

25. The method of claim 21, wherein the immunoassay is performed in the presence of one or more protein dissociating agents.

26.-29. (canceled)

30. A kit for quantifying GDF-9-BMP-15 heterodimers, the kit comprising an anti-GDF-9 antibody, an anti BMP-15 antibody, wherein the anti-GDF-9 antibody specifically binds an epitope contained in an amino acid sequence selected from SEQ ID NOS: 111, 119, 120, 123-125, 128-136, and 144-146, and wherein the anti-BMP-15 antibody specifically binds to an epitope contained in an amino acid sequence selected from SEQ ID NOS: 8-12, 28, 30, 32-34, 40, 41, 45, 46, 48-51, 55, 61, and 68.

31.-52. (canceled)

53. The method of claim 15, wherein the immunoassay is performed in the presence of one or more protein dissociating agents.


SEQUENCE LISTING (166 sequences)

SEQ ID NO: 1 - protein, 15 aa, Homo sapiens
MVLLSILRILFLCEL

SEQ ID NO: 2 - protein, 15 aa, Homo sapiens
ILRILFLCELVLFME

SEQ ID NO: 3 - protein, 15 aa, Homo sapiens
FLCELVLFMEHRAQM

SEQ ID NO: 4 - protein, 15 aa, Homo sapiens
VLFMEHRAQMAEGGQ

SEQ ID NO: 5 - protein, 15 aa, Homo sapiens
HRAQMAEGGQSSIAL

SEQ ID NO: 6 - protein, 15 aa, Homo sapiens
AEGGQSSIALLAEAP

SEQ ID NO: 7 - protein, 15 aa, Homo sapiens
SSIALLAEAPTLPLI

SEQ ID NO: 8 - protein, 15 aa, Homo sapiens
LAEAPTLPLIEELLE

SEQ ID NO: 9 - protein, 15 aa, Homo sapiens
TLPLIEELLEESPGE

SEQ ID NO: 10 - protein, 15 aa, Homo sapiens
EELLEESPGEQPRKP

SEQ ID NO: 11 - protein, 15 aa, Homo sapiens
ESPGEQPRKPRLLGH

SEQ ID NO: 12 - protein, 15 aa, Homo sapiens
QPRKPRLLGHSLRYM

SEQ ID NO: 13 - protein, 15 aa, Homo sapiens
RLLGHSLRYMLELYR

SEQ ID NO: 14 - protein, 15 aa, Homo sapiens
SLRYMLELYRRSADS

SEQ ID NO: 15 - protein, 15 aa, Homo sapiens
LELYRRSADSHGHPR

SEQ ID NO: 16 - protein, 15 aa, Homo sapiens
RSADSHGHPRENRTI

SEQ ID NO: 17 - protein, 15 aa, Homo sapiens
HGHPRENRTIGATMV

SEQ ID NO: 18 - protein, 15 aa, Homo sapiens
ENRTIGATMVRLVKP

SEQ ID NO: 19 - protein, 15 aa, Homo sapiens
GATMVRLVKPLTNVA

SEQ ID NO: 20 - protein, 15 aa, Homo sapiens
RLVKPLTNVARPHRG

SEQ ID NO: 21 - protein, 15 aa, Homo sapiens
LTNVARPHRGTWHIQ

SEQ ID NO: 22 - protein, 15 aa, Homo sapiens
RPHRGTWHIQILGFP

SEQ ID NO: 23 - protein, 15 aa, Homo sapiens
TWHIQILGFPLRPNR

SEQ ID NO: 24 - protein, 15 aa, Homo sapiens
ILGFPLRPNRGLYQL

SEQ ID NO: 25 - protein, 15 aa, Homo sapiens
LRPNRGLYQLVRATV

SEQ ID NO: 26 - protein, 15 aa, Homo sapiens
GLYQLVRATVVYRHH

SEQ ID NO: 27 - protein, 15 aa, Homo sapiens
VRATVVYRHHLQLTR

SEQ ID NO: 28 - protein, 15 aa, Homo sapiens
VYRHHLQLTRFNLSC

SEQ ID NO: 29 - protein, 15 aa, Homo sapiens
LQLTRFNLSCHVEPW

SEQ ID NO: 30 - protein, 15 aa, Homo sapiens
FNLSCHVEPWVQKNP

SEQ ID NO: 31 - protein, 15 aa, Homo sapiens
HVEPWVQKNPTNHFP

SEQ ID NO: 32 - protein, 15 aa, Homo sapiens
VQKNPTNHFPSSEGD

SEQ ID NO: 33 - protein, 15 aa, Homo sapiens
TNHFPSSEGDSSKPS

SEQ ID NO: 34 - protein, 15 aa, Homo sapiens
SSEGDSSKPSLMSNA

SEQ ID NO: 35 - protein, 15 aa, Homo sapiens
SSKPSLMSNAWKEMD

SEQ ID NO: 36 - protein, 15 aa, Homo sapiens
LMSNAWKEMDITQLV

SEQ ID NO: 37 - protein, 15 aa, Homo sapiens
WKEMDITQLVQQRFW

SEQ ID NO: 38 - protein, 15 aa, Homo sapiens
ITQLVQQRFWNNKGH

SEQ ID NO: 39 - protein, 15 aa, Homo sapiens
QQRFWNNKGHRILRL

SEQ ID NO: 40 - protein, 15 aa, Homo sapiens
NNKGHRILRLRFMCQ

SEQ ID NO: 41 - protein, 15 aa, Homo sapiens
RILRLRFMCQQQKDS

SEQ ID NO: 42 - protein, 15 aa, Homo sapiens
RFMCQQQKDSGGLEL

SEQ ID NO: 43 - protein, 15 aa, Homo sapiens
QQKDSGGLELWHGTS

SEQ ID NO: 44 - protein, 15 aa, Homo sapiens
GGLELWHGTSSLDIA

SEQ ID NO: 45 - protein, 15 aa, Homo sapiens
WHGTSSLDIAFLLLY

SEQ ID NO: 46 - protein, 15 aa, Homo sapiens
SLDIAFLLLYFNDTH

SEQ ID NO: 47 - protein, 15 aa, Homo sapiens
FLLLYFNDTHKSIRK

SEQ ID NO: 48 - protein, 15 aa, Homo sapiens
FNDTHKSIRKAKFLP

SEQ ID NO: 49 - protein, 15 aa, Homo sapiens
KSIRKAKFLPRGMEE

SEQ ID NO: 50 - protein, 15 aa, Homo sapiens
AKFLPRGMEEFMERE

SEQ ID NO: 51 - protein, 15 aa, Homo sapiens
RGMEEFMERESLLRR

SEQ ID NO: 52 - protein, 15 aa, Homo sapiens
FMERESLLRRTRQAD

SEQ ID NO: 53 - protein, 15 aa, Homo sapiens
SLLRRTRQADGISAE

SEQ ID NO: 54 - protein, 15 aa, Homo sapiens
TRQADGISAEVTASS

SEQ ID NO: 55 - protein, 15 aa, Homo sapiens
GISAEVTASSSKHSG

SEQ ID NO: 56 - protein, 15 aa, Homo sapiens
VTASSSKHSGPENNQ

SEQ ID NO: 57 - protein, 15 aa, Homo sapiens
SKHSGPENNQCSLHP

SEQ ID NO: 58 - protein, 15 aa, Homo sapiens
PENNQCSLHPFQISF

SEQ ID NO: 59 - protein, 15 aa, Homo sapiens
CSLHPFQISFRQLGW

SEQ ID NO: 60 - protein, 15 aa, Homo sapiens
FQISFRQLGWDHWII

SEQ ID NO: 61 - protein, 15 aa, Homo sapiens
RQLGWDHWIIAPPFY

SEQ ID NO: 62 - protein, 15 aa, Homo sapiens
DHWIIAPPFYTPNYC

SEQ ID NO: 63 - protein, 15 aa, Homo sapiens
APPFYTPNYCKGTCL

SEQ ID NO: 64 - protein, 15 aa, Homo sapiens
TPNYCKGTCLRVLRD

SEQ ID NO: 65 - protein, 15 aa, Homo sapiens
KGTCLRVLRDGLNSP

SEQ ID NO: 66 - protein, 15 aa, Homo sapiens
RVLRDGLNSPNHAII

SEQ ID NO: 67 - protein, 15 aa, Homo sapiens
GLNSPNHAIIQNLIN

SEQ ID NO: 68 - protein, 15 aa, Homo sapiens
NHAIIQNLINQLVDQ

SEQ ID NO: 69 - protein, 15 aa, Homo sapiens
QNLINQLVDQSVPRP

SEQ ID NO: 70 - protein, 15 aa, Homo sapiens
QLVDQSVPRPSCVPY

SEQ ID NO: 71 - protein, 15 aa, Homo sapiens
SVPRPSCVPYKYVPI

SEQ ID NO: 72 - protein, 15 aa, Homo sapiens
SCVPYKYVPISVLMI

SEQ ID NO: 73 - protein, 15 aa, Homo sapiens
KYVPISVLMIEANGS

SEQ ID NO: 74 - protein, 15 aa, Homo sapiens
SVLMIEANGSILYKE

SEQ ID NO: 75 - protein, 15 aa, Homo sapiens
EANGSILYKEYEGMI

SEQ ID NO: 76 - protein, 15 aa, Homo sapiens
ILYKEYEGMIAESCT

SEQ ID NO: 77 - protein, 15 aa, Homo sapiens
YKEYEGMIAESCTCR

SEQ ID NO: 78 - protein, 15 aa, Homo sapiens
MARPNKFLLWFCCFA

SEQ ID NO: 79 - protein, 15 aa, Homo sapiens
KFLLWFCCFAWLCFP

SEQ ID NO: 80 - protein, 15 aa, Homo sapiens
FCCFAWLCFPISLGS

SEQ ID NO: 81 - protein, 15 aa, Homo sapiens
WLCFPISLGSQASGG

SEQ ID NO: 82 - protein, 15 aa, Homo sapiens
ISLGSQASGGEAQIA

SEQ ID NO: 83 - protein, 15 aa, Homo sapiens
QASGGEAQIAASAEL

SEQ ID NO: 84 - protein, 15 aa, Homo sapiens
EAQIAASAELESGAM

SEQ ID NO: 85 - protein, 15 aa, Homo sapiens
ASAELESGAMPWSLL

SEQ ID NO: 86 - protein, 15 aa, Homo sapiens
ESGAMPWSLLQHIDE

SEQ ID NO: 87 - protein, 15 aa, Homo sapiens
PWSLLQHIDERDRAG

SEQ ID NO: 88 - protein, 15 aa, Homo sapiens
QHIDERDRAGLLPAL

SEQ ID NO: 89 - protein, 15 aa, Homo sapiens
RDRAGLLPALFKVLS

SEQ ID NO: 90 - protein, 15 aa, Homo sapiens
LLPALFKVLSVGRGG

SEQ ID NO: 91 - protein, 15 aa, Homo sapiens
FKVLSVGRGGSPRLQ

SEQ ID NO: 92 - protein, 15 aa, Homo sapiens
VGRGGSPRLQPDSRA

SEQ ID NO: 93 - protein, 15 aa, Homo sapiens
SPRLQPDSRALHYMK

SEQ ID NO: 94 - protein, 15 aa, Homo sapiens
PDSRALHYMKKLYKT

SEQ ID NO: 95 - protein, 15 aa, Homo sapiens
LHYMKKLYKTYATKE

SEQ ID NO: 96 - protein, 15 aa, Homo sapiens
KLYKTYATKEGIPKS

SEQ ID NO: 97 - protein, 15 aa, Homo sapiens
YATKEGIPKSNRSHL

SEQ ID NO: 98 - protein, 15 aa, Homo sapiens
GIPKSNRSHLYNTVR

SEQ ID NO: 99 - protein, 15 aa, Homo sapiens
NRSHLYNTVRLFTPC

SEQ ID NO: 100 - protein, 15 aa, Homo sapiens
YNTVRLFTPCTRHKQ

SEQ ID NO: 101 - protein, 15 aa, Homo sapiens
LFTPCTRHKQAPGDQ

SEQ ID NO: 102 - protein, 15 aa, Homo sapiens
TRHKQAPGDQVTGIL

SEQ ID NO: 103 - protein, 15 aa, Homo sapiens
APGDQVTGILPSVEL

SEQ ID NO: 104 - protein, 15 aa, Homo sapiens
VTGILPSVELLFNLD

SEQ ID NO: 105 - protein, 15 aa, Homo sapiens
PSVELLFNLDRITTV

SEQ ID NO: 106 - protein, 15 aa, Homo sapiens
LFNLDRITTVEHLLK

SEQ ID NO: 107 - protein, 15 aa, Homo sapiens
RITTVEHLLKSVLLY

SEQ ID NO: 108 - protein, 15 aa, Homo sapiens
EHLLKSVLLYNINNS

SEQ ID NO: 109 - protein, 15 aa, Homo sapiens
SVLLYNINNSVSFSS

SEQ ID NO: 110 - protein, 15 aa, Homo sapiens
NINNSVSFSSAVKCV

SEQ ID NO: 111 - protein, 15 aa, Homo sapiens
VSFSSAVKCVCNLMI

SEQ ID NO: 112 - protein, 15 aa, Homo sapiens
AVKCVCNLMIKEPKS

SEQ ID NO: 113 - protein, 15 aa, Homo sapiens
CNLMIKEPKSSSRTL

SEQ ID NO: 114 - protein, 15 aa, Homo sapiens
KEPKSSSRTLGRAPY

SEQ ID NO: 115 - protein, 15 aa, Homo sapiens
SSRTLGRAPYSFTFN

SEQ ID NO: 116 - protein, 15 aa, Homo sapiens
GRAPYSFTFNSQFEF

SEQ ID NO: 117 - protein, 15 aa, Homo sapiens
SFTFNSQFEFGKKHK

SEQ ID NO: 118 - protein, 15 aa, Homo sapiens
SQFEFGKKHKWIQID

SEQ ID NO: 119 - protein, 15 aa, Homo sapiens
GKKHKWIQIDVTSLL

SEQ ID NO: 120 - protein, 15 aa, Homo sapiens
WIQIDVTSLLQPLVA

SEQ ID NO: 121 - protein, 15 aa, Homo sapiens
VTSLLQPLVASNKRS

SEQ ID NO: 122 - protein, 15 aa, Homo sapiens
QPLVASNKRSIHMSI

SEQ ID NO: 123 - protein, 15 aa, Homo sapiens
SNKRSIHMSINFTCM

SEQ ID NO: 124 - protein, 15 aa, Homo sapiens
IHMSINFTCMKDQLE

SEQ ID NO: 125 - protein, 15 aa, Homo sapiens
NFTCMKDQLEHPSAQ

SEQ ID NO: 126 - protein, 15 aa, Homo sapiens
KDQLEHPSAQNGLFN

SEQ ID NO: 127 - protein, 15 aa, Homo sapiens
HPSAQNGLFNMTLVS

SEQ ID NO: 128 - protein, 15 aa, Homo sapiens
NGLFNMTLVSPSLIL

SEQ ID NO: 129 - protein, 15 aa, Homo sapiens
MTLVSPSLILYLNDT

SEQ ID NO: 130 - protein, 15 aa, Homo sapiens
PSLILYLNDTSAQAY

SEQ ID NO: 131 - protein, 15 aa, Homo sapiens
YLNDTSAQAYHSWYS

SEQ ID NO: 132 - protein, 15 aa, Homo sapiens
SAQAYHSWYSLHYKR

SEQ ID NO: 133 - protein, 15 aa, Homo sapiens
HSWYSLHYKRRPSQG

SEQ ID NO: 134 - protein, 15 aa, Homo sapiens
LHYKRRPSQGPDQER

SEQ ID NO: 135 - protein, 15 aa, Homo sapiens
RPSQGPDQERSLSAY

SEQ ID NO: 136 - protein, 15 aa, Homo sapiens
PDQERSLSAYPVGEE

SEQ ID NO: 137 - protein, 15 aa, Homo sapiens
SLSAYPVGEEAAEDG

SEQ ID NO: 138 - protein, 15 aa, Homo sapiens
PVGEEAAEDGRSSHH

SEQ ID NO: 139 - protein, 15 aa, Homo sapiens
AAEDGRSSHHRHRRG

SEQ ID NO: 140 - protein, 15 aa, Homo sapiens
RSSHHRHRRGQETVS

SEQ ID NO: 141 - protein, 15 aa, Homo sapiens
RHRRGQETVSSELKK

SEQ ID NO: 142 - protein, 15 aa, Homo sapiens
QETVSSELKKPLGPA

SEQ ID NO: 143 - protein, 15 aa, Homo sapiens
SELKKPLGPASFNLS

SEQ ID NO: 144 - protein, 15 aa, Homo sapiens
PLGPASFNLSEYFRQ

SEQ ID NO: 145 - protein, 15 aa, Homo sapiens
SFNLSEYFRQFLLPQ

SEQ ID NO: 146 - protein, 15 aa, Homo sapiens
EYFRQFLLPQNECEL

SEQ ID NO: 147 - protein, 15 aa, Homo sapiens
FLLPQNECELHDFRL

SEQ ID NO: 148 - protein, 15 aa, Homo sapiens
NECELHDFRLSFSQL

SEQ ID NO: 149 - protein, 15 aa, Homo sapiens
HDFRLSFSQLKWDNW

SEQ ID NO: 150 - protein, 15 aa, Homo sapiens
SFSQLKWDNWIVAPH

SEQ ID NO: 151 - protein, 15 aa, Homo sapiens
KWDNWIVAPHRYNPR

SEQ ID NO: 152 - protein, 15 aa, Homo sapiens
IVAPHRYNPRYCKGD

SEQ ID NO: 153 - protein, 15 aa, Homo sapiens
RYNPRYCKGDCPRAV

SEQ ID NO: 154 - protein, 15 aa, Homo sapiens
YCKGDCPRAVGHRYG

SEQ ID NO: 155 - protein, 15 aa, Homo sapiens
CPRAVGHRYGSPVHT

SEQ ID NO: 156 - protein, 15 aa, Homo sapiens
GHRYGSPVHTMVQNI

SEQ ID NO: 157 - protein, 15 aa, Homo sapiens
SPVHTMVQNIIYEKL

SEQ ID NO: 158 - protein, 15 aa, Homo sapiens
MVQNIIYEKLDSSVP

SEQ ID NO: 159 - protein, 15 aa, Homo sapiens
IYEKLDSSVPRPSCV

SEQ ID NO: 160 - protein, 15 aa, Homo sapiens
DSSVPRPSCVPAKYS

SEQ ID NO: 161 - protein, 15 aa, Homo sapiens
RPSCVPAKYSPLSVL

SEQ ID NO: 162 - protein, 15 aa, Homo sapiens
PAKYSPLSVLTIEPD

SEQ ID NO: 163 - protein, 15 aa, Homo sapiens
PLSVLTIEPDGSIAY

SEQ ID NO: 164 - protein, 15 aa, Homo sapiens
TIEPDGSIAYKEYED

SEQ ID NO: 165 - protein, 15 aa, Homo sapiens
GSIAYKEYEDMIATK

SEQ ID NO: 166 - protein, 15 aa, Homo sapiens
YKEYEDMIATKCTCR